(19) United States
(12) Patent Application Publication
(10) Pub. No.: US 2022/0133697 A1
(43) Pub. Date: May 5, 2022

(54) INJECTABLE FORMULATIONS

(71) Applicant: Aldeyra Therapeutics, Inc., Lexington, MA (US)

(72) Inventors: Stephen Gitu MACHATHA, Wilmington, MA (US); Susan MACDONALD, Danvers, MA (US)

(21) Appl. No.: 17/299,731

(22) PCT Filed: Dec. 5, 2019
(86) PCT No.: PCT/US2019/064669

§ 371 Date: Jun. 3, 2021

Related U.S. Application Data
(60) Provisional application No. 62/775,779, filed on Dec. 5, 2018.

(51) Int. Cl. A61K 31/423; A61K 9/00; A61K 9/127; A61K 47/02; A61K 47/40; A61K 47/24; A61K 47/28; A61K 9/16; A61K 9/50; A61P 27/02

(52) U.S. Cl. CPC A61K 31/423; A61K 9/0019; A61K 9/127; A61K 47/02; A61K 47/40; A61P 27/02; A61K 47/28; A61K 9/1647; A61K 9/1611; A61K 9/5031; A61K 47/24

(57) ABSTRACT

The present disclosure relates to therapeutic compounds and formulations for treating, reducing the risk of, or for preventing a disease, disorder, or condition characterized by the presence of toxic aldehydes.

### 1. CROSS-REFERENCE TO RELATED APPLICATIONS

This application claims the benefit under 35 U.S.C. § 119(e) of U.S. Provisional Application No. 62/775,779, filed Dec. 5, 2018, the contents of all of which are incorporated herein in their entireties by reference thereto.

### 2. BACKGROUND

Metabolic and inflammatory processes in cells can result in formation of toxic aldehydes, such as malondialdehyde (MDA), 4-hydroxy-2-nonenal (HNE or 4-NE), glyoxal, and acetaldehyde. MDA, HINE and other toxic aldehydes are generated by a myriad of metabolic mechanisms involving fatty alcohols, sphingolipids, glycolipids, phytols, fatty acids, arachidonic acid metabolism (Rizzo et al., 2007, Mol Genet Metab. 90(1):1-9), polyamine metabolism (Wood et al., 2006, Brain Res. 1122(1):184-90), lipid peroxidation, oxidative metabolism (Buddi et al., 2002, Histochem Cytochem. 50(3):341-51; Zhou et al., 2005, Exp Eye Res. 80(4):567-80; Zhou et al., 2005, J Biol Chem. 280(27):25377-82), and glucose metabolism (Pozzi et al., 2009, J Am Soc Nephrol. 20(10):2119-25).

Aldehydes are highly reactive with proteins, carbohydrates, lipids and DNA, leading to chemically modified biological molecules; activation of inflammatory mediators such as NF-kappaB; and damage in diverse tissues. They can react with primary amino groups and other chemical moieties on proteins, phospholipids, carbohydrates, and DNA, leading in many cases to adverse biological consequences, such as mutagenesis and carcinogenesis (Marnett, 2002, Toxicology 181-182:219-22) and acute or chronic inflammation. Given the implication of toxic aldehydes in numerous disease indications, there is a need for therapeutic agents and pharmaceutical compositions for treating, preventing, and/or reducing the risk of diseases, disorders or conditions associated with the presence of toxic aldehydes.

### 3. SUMMARY

The present disclosure provides a pharmaceutical composition of a compound, which reacts with aldehydes, for treating diseases, disorders, and conditions characterized by the presence of toxic aldehydes. In some embodiments, one or more of toxic aldehydes are increased or elevated in these diseases, disorders, or conditions. In particular, the pharmaceutical compositions are injectable compositions, for example compositions which can be administered subcutaneously, intradermally, intraocularly (e.g., intravitreal or periocular), intramuscularly, intraperitoneally, or in some embodiments, intravenously. In some embodiments, the pharmaceutical compositions are depot formulations.

In one aspect, the pharmaceutical composition comprises a compound of formula I:

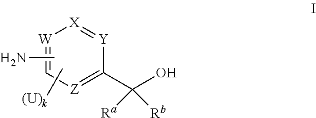

or a pharmaceutically acceptable salt thereof, wherein:

each W, X, Y, or Z is independently selected from N, O, S, CU, CH and C—NH₂, wherein one of W, X, Y, or Z is C—NH₂;

R^{a} is C₁₋₄ aliphatic optionally substituted with 1, 2, or 3 deuterium or halogen atoms;

R^{b} is C₁₋₄ aliphatic optionally substituted with 1, 2, or 3 deuterium or halogen atoms; or R and R, taken together with the carbon atom to which they are attached, form a 3- to 8-membered cycloalkyl or heterocyclyl ring containing 1-2 heteroatoms selected from nitrogen, oxygen, and sulfur;

k is 0, 1, 2, 3, or 4;

each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;

two occurrences of U on adjacent carbon atoms can form an optionally substituted fused ring, selected from a fused phenyl ring; a fused 5- to 6-membered saturated or partially unsaturated heterocyclic ring containing 1-3 heteroatoms independently selected from nitrogen, oxygen, and sulfur; and a fused 5- to 6-membered heteroaryl ring containing 1-3 heteroatoms independently selected from nitrogen, oxygen, and sulfur; and

each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In some embodiments, the pharmaceutical compositions comprise a compound of formula II:

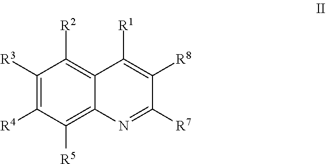

or a pharmaceutically acceptable salt thereof, wherein:

each of R¹, R⁷, and R⁸ is independently H, D, halogen, —NH₂, —CN, —OR, —SR, optionally substituted C₁₋₆ aliphatic, or

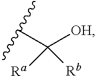

wherein one of R¹, R⁷ and R⁸ is —NH₂ and other one of R¹ R⁷ and R⁸ is

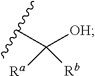

R² is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;

R³ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;

R⁴ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;

R⁵ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;

R^{a} is C₁₋₄ aliphatic optionally substituted with 1, 2, or 3 deuterium or halogen atoms;

R^{b} is C₁₋₄ aliphatic optionally substituted with 1, 2, or 3 deuterium or halogen atoms; or R^{a} and R^{b}, taken together with the carbon atom to which they are attached, form a 3- to 8-membered cycloalkyl or heterocyclyl ring containing 1-2 heteroatoms selected from nitrogen, oxygen, and sulfur; and

each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur: a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In some embodiments, the pharmaceutical composition comprises a compound of formula III:

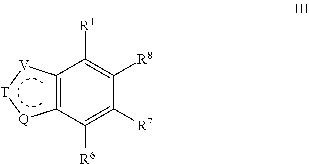

or a pharmaceutically acceptable salt thereof, wherein:

Q, T and V are independently S, N, O, or —C—R;

each of R¹, R⁶, R⁷, and R⁸ is independently H, D, halogen, —NH₂—CN, —OR, —SR, optionally substituted C₁₋₆ aliphatic, or

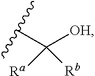

wherein one of R¹, R⁶, R⁷, and R⁸ is —NH₂ and other one of R¹, R⁶, R⁷ and R⁸ is

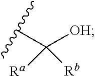

R^{a} is C₁₋₄ aliphatic optionally substituted with 1, 2, or 3 deuterium or halogen atoms; and

R^{b} is C₁₋₄ aliphatic optionally substituted with 1, 2, or 3 deuterium or halogen atoms; or

R^{a} and R^{b}, taken together with the carbon atom to which they are attached, form a 3-8 membered cycloalkyl or heterocyclyl ring containing 1-2 heteroatoms selected from nitrogen, oxygen, and sulfur; and

each R is independently selected from hydrogen, deuterium, and an optionally substituted group selected from: C₁₋₆ aliphatic, a 3- to 8-membered saturated or partially unsaturated monocyclic carbocyclic ring, phenyl, an 8- to 10-membered bicyclic aryl ring, a 3- to 8-membered saturated or partially unsaturated monocyclic heterocyclic ring having 1-4 heteroatoms independently selected from nitrogen, oxygen, and sulfur, a 5- to 6-membered monocyclic heteroaryl ring having 1-4 heteroatoms independently selected from nitrogen, oxygen, and sulfur, a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In some embodiments, the pharmaceutical composition comprises a compound of formula I-22:

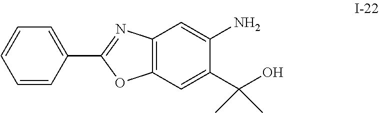 [SMILES: CC(C)(O)c1cc2oc(-c3ccccc3)nc2cc1N]

or a pharmaceutically acceptable salt thereof.

In some embodiments, the pharmaceutical composition comprises a compound of formula I-5:

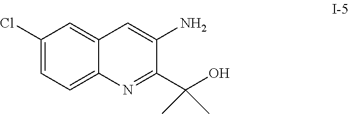 [SMILES: CC(C)(O)c1nc2ccc(Cl)cc2cc1N]

or a pharmaceutically acceptable salt thereof.

In some embodiments, pharmaceutical composition comprises a compound of formula I-6:

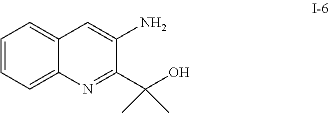

or a pharmaceutically acceptable salt thereof.

In one aspect, the pharmaceutical composition comprises the compound and a viscosity enhancing agent. In some embodiments, the pharmaceutical composition has a viscosity of between 1 kcP and 200 kcP. In some embodiments, the pharmaceutical composition has a viscosity enhancing agent selected from hyaluronate, hyaluronic acid or pharmaceutical acceptable salts thereof, cross-linked hyaluronic acid, polyvinylpyrrolidone (PVP), hydroxypropyl methylcellulose, hydroxypropyl cellulose, hydroxylethyl cellulose, glycerol, and mixtures thereof.

In some embodiments, the viscosity enhancing agent is hyaluronate, hyaluronic acid or a pharmaceutically acceptable salt thereof. In some embodiments, the hyaluronate or hyaluronic acid or a pharmaceutically acceptable salt thereof has a molecular weight of about 500,000 to about 5×10⁶ Daltons.

In some embodiments, the pharmaceutical composition comprising the compound and a viscosity enhancing agent further comprises one or more excipients selected from a tonicity agent, buffering agent, chelating agent, surfactant, preservative, and antioxidant.

In another aspect, the pharmaceutical composition comprises the compound formulated in a liposome. In some embodiments, the liposome is a submicron-sized liposome, for example an average particle size of about 20 nm to about 1 um or less.

In some embodiments, the liposomes containing the compound includes one or more of phosphatidylcholines, phosphatidylethanolamines, phosphatidic acids, gangliosides, glycolipids, phosphatidylglycerols, and cholesterol. In some embodiments, the liposome comprises at least one phosphatidylcholine selected from L-α-phosphatidylcholine, egg phosphatidylcholines (EPC). 1,2-dioleoyl-sn-glycero-3-phosphocholines (DOPC), 1,2-dioleoyl-sn-glycero-O-ethyl-3-phosphocholines, 1,2-dilauroyl-sn-glycero-3-phosphocholines (DLPC), 1,2-dimyristoyl-sn-glycero-3-phosphocholines (DMPC), 1,2-dipalmitoyl-sn-glycero-3-phosphocholines (DPPC), 1,2-distearoyl-sn-glycero-3-phosphocholines (DSPC) and mixtures thereof. In some embodiments, in addition to the phosphatidylcholine or derivative thereof, the liposome further includes cholesterol or a derivative thereof suitable for forming liposomes.

In some embodiments, the liposome comprises (a) 1,2-dipalmitoyl-sn-glycero-3-phosphocholine (DPPC); (b) egg phosphatidylcholine (EPC) and 1-α-distearoyl phosphatidylcholine (DSPC); (c) egg phosphatidylcholine (EPC) or 1,2-dipalmitoyl-sn-glycero-3-phosphocholine (DPPC), and cholesterol or a derivative thereof; (d) dioleoylphosphatidylcholine (DOPC), dioleoylphosphatidylglycerol (DOPG), and cholesterol; or (e) dipalmitoylphosphatidylcholine (DPPC), palmitoyl-oleoylphosphatidylcholine (POPC), and cholesterol.

In another aspect, the pharmaceutical composition comprises the compound formulated in a microparticle or nanoparticle, wherein the microparticle or nanoparticle comprises a biodegradable polymer.

In some embodiments, the microparticle or nanoparticle comprises a biodegradable polymer selected from poly(ester)s, poly(ester amide)s, poly(anhydride)s, poly(carbonate)s, poly(amino acid)s, poly(amide)s, poly(urethane)s, poly(ortho-ester)s, poly(iminocarbonate)s, poly(phosphazene)s, and combinations thereof.

In some embodiments, the biodegradable polymer comprises poly(lactide-co-glycolide) (PLGA). In some embodiments, the biodegradable polymer comprises poly(ester amide)s.

In some embodiments, the pharmaceutical composition comprises the compound formulated in a microparticle, wherein the microparticle comprises a biodegradable polymer. In some embodiments, the microparticle has an average particle size of about 1 um to about 250 um.

In some embodiments, the pharmaceutical composition comprises the compound formulated in a nanoparticle, wherein the nanoparticle comprises a biodegradable polymer. In some embodiments, the nanoparticle has an average particle size of less than 1 um.

In a further aspect, the pharmaceutical composition comprises the compound formulated in a calcium phosphate particle.

In some embodiments, the calcium phosphate particle comprises tricalcium phosphate (β-TCP) or hydroxyapatite. In some embodiments, the calcium phosphate particle has an average particle size of less than 1 um.

In some embodiments, the calcium phosphate particle has a coating of polyethylene glycol or lipid. In some embodiments, the compound is disposed or absorbed on the surface of the calcium phosphate particle.

In another aspect, the pharmaceutical composition comprises the compound and a complexing agent selected from a polyamino acid, galactomannan polymer or cationic galactomannan polymer, cellulosic polymer, quaternary ammonium polymer, and combinations thereof.

In some embodiments, the complexing agent is a polyamino acid. In some embodiments, the polyamino acid is poly-L-lysine.

In some embodiments, the complexing agent is a galactomannan polymer or cationic galactomannan polymer.

In another aspect, the pharmaceutical composition comprises the compound and a cyclodextrin, wherein the composition is suitable for parenteral administration, particularly for intraocular administration.

In some embodiments, the cyclodextrin is selected from α-cyclodextrin, β-cyclodextrin, γ-cyclodextrin, derivatives thereof, and combinations thereof. In some embodiments, the cyclodextrin is selected from carboxyalkyl cyclodextrin, hydroxyalkyl cyclodextrin, sulfoalkylether cyclodextrin, and alkyl cyclodextrin.

In some embodiments, the cyclodextrin is a β-cyclodextrin. In some embodiments, the β-cyclodextrin is hydroxyalkyl-β-cyclodextrin or sulfoalkylether-β-cyclodextrin.

In a further aspect, the pharmaceutical composition comprises the compound formulated in a hydrogel. In some embodiments, the hydrogel is a thermoresponsive, ionic responsive, or a pH responsive hydrogel.

In some embodiments, the hydrogel is comprised of polyethylene oxide; polypropylene oxide; polyoxyethylene-polyoxypropylene block copolymers (e.g., Poloxamer 407 and Poloxamer 188); acrylic polymers such as, but not limited to, carbomers (e.g., Carbopol® 974P); polyvinylpyrroidones; polyethylene glycols (PEGs); gelatin, polyvinyl alcohols (PVA); polyhydroxy ethyl methacrylate (poly-HEMA or PHEMA); celluloses; alginates; chitins or combinations thereof.

In some embodiments, the foregoing pharmaceutical compositions further comprises one or more excipients selected from a tonicity agent, viscosity enhancing agent, buffering agent, chelating agent, surfactant, preservative, and antioxidant.

In another aspect, the pharmaceutical compositions are used in treating a disease, disorder, or condition characterized by an increase or elevated level of a toxic aldehyde.

In some embodiments, the pharmaceutical compositions are used to treat, reduce the risk of or prevent the occurrence of an ocular disease, disorder or condition characterized by an increase or elevated level of toxic aldehydes. In some embodiments, the ocular disease, disorder, or condition is characterized by the presence of a pathological inflammatory response.

In some embodiments, the ocular disease, disorder or condition includes macular degeneration, including age-related macular degeneration (AMD), wet age-related macular degeneration, dry age-related macular degeneration: Stargardt's disease; dry eye syndrome or disease; cataracts; keratoconus; bullous and other keratopathy; Fuch's endothelial dystrophy; allergic conjunctivitis: ocular cicatricial pemphigoid: lacrimal gland dysfunction; uveitis, including anterior uveitis, posterior uveitis, and pan-uveitis; scleritis; ocular Stevens-Johnson Syndrome; ocular rosacea, with or without meibomian gland dysfunction; macular edema, including diabetic macular edema (DME), non-clinically significant macular edema (Non-CSME), and clinically significant macular edema (CSME); endophthalmitis; and inflammation and/or fibrosis associated with ocular injury.

In some embodiments, the ocular disease, disorder, or condition for treatment or prevention with the pharmaceutical compositions is inflammation and/or fibrosis associated with ocular injury, such as from trauma to the eye or eye surgery.

In some embodiments, the pharmaceutical composition is used to treat, reduce the risk of or prevent the occurrence of non-ocular diseases, disorders, or conditions characterized by an increase or elevated level of toxic aldehydes. In some embodiments, the non-ocular diseases, disorders, or conditions having increased levels of toxic aldehydes include non-ocular inflammatory diseases or disorders; autoimmune diseases or disorders; neurological or neurodegenerative diseases or disorders; chronic liver diseases or disorders; chronic obstructive lung diseases or disorders, and non-ocular fibrotic diseases or disorders.

In some embodiments, the disease, disorder or condition for treatment with the pharmaceutical compositions is a genetic condition having a defect in aldehyde metabolism and resulting in an increase or elevated level of a toxic aldehyde. In some embodiments, the genetic disease, disorder or condition is Sjögren-Larsson Syndrome or succinic semialdehyde dehydrogenase (SSADH) deficiency.

### BRIEF DESCRIPTION OF THE FIGURES

FIG. 1A and FIG. 1B show total ocular inflammation scores in endotoxin induced uveitis in rats treated topically with vehicle, Compound I-5, or Compound I-22.

FIG. 2A and FIG. 2B show anterior chamber inflammation scores in endotoxin induced uveitis in rats treated topically with vehicle, Compound I-5, or Compound I-22.

FIG. 3A and FIG. 3B show retina-choroid inflammation scores in endotoxin induced uveitis in rats treated topically with vehicle, Compound I-5, or Compound I-22.

FIG. 4A and FIG. 4B show total ocular inflammation scores in endotoxin induced uveitis in rats treated intravitreally with vehicle, Compound I-5, or Compound I-22.

FIG. 5A and FIG. 5B show anterior chamber inflammation scores in endotoxin induced uveitis in rats treated intravitreally with vehicle, Compound I-5, or Compound I-22.

FIG. 6A and FIG. 6B show retina-choroid inflammation scores in endotoxin induced uveitis in rats treated intravitreally with vehicle, Compound I-5, or Compound I-22.

FIG. 7 shows effect of intravitreal administration of Compound I-5, and Compound I-22 on formation of A2E in the retinas of abcr−/− mice.

FIG. 8 shows retinal thickness in untreated control animals or animals with chemically induced diabetes treated by intravitreal administration of vehicle or Compound I-22.

FIG. 9 shows effect on neutrophil infiltration in the eye of untreated control animals or eye of animals with chemically induced diabetes treated by intravitreal administration of vehicle or Compound I-22.

FIG. 10 shows effect on retinal vascularity in the eye of untreated control animals or eye of animals with chemically induced diabetes treated by intravitreal administration of vehicle or Compound I-22.

### 4. DETAILED DESCRIPTION

The present disclosure provides pharmaceutical compositions of compounds having aldehyde trapping activity. The pharmaceutical compositions are injectable compositions, particularly parenteral formulations, such as for subcutaneous, intradermal, intramuscular, intraocular (e.g., intravitreal), and intraperitoneal administration. In certain embodiments, the compositions, where suitable, are formulated for intravenous administration. In some embodiments, the pharmaceutical compositions are used for treating diseases, disorders, or conditions characterized by the presence of toxic aldehydes. In some embodiments, the pharmaceutical compositions are used for treating diseases, disorders, or conditions characterized by a pathological inflammatory reaction.

### 4.1. Definitions

Compounds of the present disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Smith, M. B. and March, J., eds., John Wiley & Sons, New York (2001), the entire contents of which are hereby incorporated by reference.

“Aliphatic” or “aliphatic group”, as used herein, means a straight-chain (i.e., unbranched) or branched, substituted or unsubstituted hydrocarbon chain that is completely saturated or that contains one or more units of unsaturation, or a monocychc hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

“Lower alkyl” refers to a C₁₋₄ straight or branched alkyl group. Exemplary lower alkyl groups are methyl, ethyl, propyl, isopropyl, butyl, isobutyl, and tert-butyl.

“Lower haloalkyl” refers to a C₁₋₄ straight or branched alkyl group that is substituted with one or more halogen atoms.

“Heteroatom” means one or more of oxygen, sulfur, nitrogen, phosphorus, or silicon (including, any oxidized form of nitrogen, sulfur, phosphorus, or silicon: the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

“Unsaturated,” as used herein, means that a moiety has one or more units of unsaturation.

“Bivalent C₁₋₈ (or C₁₋₆) saturated or unsaturated, straight or branched, hydrocarbon chain”, refers to bivalent alkylene, alkenylene, and alkynylene chains that are straight or branched as defined herein.

“Alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

“Alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

“Halogen” means F, Cl, Br, or I.

“Aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.”

“Aryl” used alone or as pail of a larger moiety as in “aralkyl,” “aralkoxy,” or “aryloxyalkyl,” refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

“Heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 r electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

“Heterocycle,” “heterocyclyl,” “heterocyclic radical,” and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

“Partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen, —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}: —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)R^{∘}; —N(R^{∘})N(R^{∘})C(O)R^{∘}; —N(R^{∘})N(R^{∘})C(O)NR^{∘}₂; —N(R))N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂B)₀₋₄SSR^{∘}; —(CH₂)₀₋₄S(O)₂R^{∘}; —(CH₂)₀₋₄S(O)₂R^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}—, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

“Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure, for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

“Bioerodible polymer” refers to a polymer which degrades in vivo. Erosion of the polymer over time results in release of an active agent. By way of example and not limitation, hydrogels such as methylcellulose which act to release drug through polymer swelling are specifically excluded from the term “bioerodible (or biodegradable) polymer. The words “bioerodible” and “biodegradable” are synonymous and are used interchangeably herein.

“Pharmaceutically acceptable” means, for example, a carrier, diluent or excipient that is compatible with the other ingredients of the formulation and generally safe for administration to a recipient thereof or that does not cause an undesired adverse physical reaction upon administration.

### 4.2. Compounds for the Pharmaceutical Compositions

In one aspect, the pharmaceutical composition herein, such as for parenteral formulations, comprises a compound of formula I:

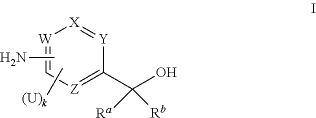

or a pharmaceutically acceptable salt thereof, wherein:

each W, X, Y, or Z is independently selected from N, O, S, CU, CH and C—NH₂, wherein one of W, X, Y, or Z is C—NH₂;

R^{a} is C₁₋₄ aliphatic optionally substituted with 1, 2, or 3 deuterium or halogen atoms;

R^{b} is C₁₋₄ aliphatic optionally substituted with 1, 2, or 3 deuterium or halogen atoms; or R^{a} and R^{b}, taken together with the carbon atom to which they are attached, form a 3- to 8-membered cycloalkyl or heterocyclyl ring containing 1-2 heteroatoms selected from nitrogen, oxygen, and sulfur;

k is 0, 1, 2, 3, or 4;

each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;

two occurrences of U on adjacent carbon atoms can form an optionally substituted fused ring, selected from a fused phenyl ring; a fused 5- to 6-membered saturated or partially unsaturated heterocyclic ring containing 1-3 heteroatoms independently selected from nitrogen, oxygen, and sulfur; and a fused 5- to 6-membered heteroaryl ring containing 1-3 heteroatoms independently selected from nitrogen, oxygen, and sulfur; and

each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In some embodiments, the —C—NH₂ and —CR^{a}R^{b}OH groups are on adjacent carbon atoms of the depicted ring of formula I.

As defined above and described herein, W is independently selected from N, O, S, CU, CH and C—NH₂. In some embodiments, W is N. In some embodiments, W is O. In some embodiments, W is S. In some embodiments, W is CU. In some embodiments, W is CH. In some embodiments, W is C—NH₂.

As defined above and described herein, X is independently selected from N, O, S, CU, CH and C—NH₂. In some embodiments, X is N. In some embodiments, X is O. In some embodiments, X is S. In some embodiments, X is CU. In some embodiments, X is CH. In some embodiments, X is C—NH₂.

As defined above and described herein, Y is independently selected from N, O, S, CU, CH and C—NH₂. In some embodiments, Y is N. In some embodiments, Y is O. In some embodiments, Y is S. In some embodiments, Y is CU. In some embodiments, Y is CH. In some embodiments, Y is C—NH₂.

As defined above and described herein, Z is independently selected from N, O, S, CU, CH and C—NH₂. In some embodiments, Z is N. In some embodiments, Z is O. In some embodiments, Z is S. In some embodiments, Z is CU. In some embodiments, Z is CH. In some embodiments, Z is C—NH₂.

As defined above and described herein, k is 0, 1, 2, 3, or 4. In some embodiments k is 0. In some embodiments, k is 1. In some embodiments, k is 2. In some embodiments, k is 3. In some embodiments, k is 4.

As defined above and described herein, each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, U is halogen. In some embodiments, U is fluorine. In some embodiments, U is chlorine. In some embodiments, U is bromine.

In some embodiments, U is —R. In some embodiments, U is hydrogen. In some embodiments, U is deuterium. In some embodiments, U is optionally substituted C₁₋₆ aliphatic. In some embodiments, U is an optionally substituted 3- to 8-membered saturated or partially unsaturated monocyclic carbocyclic ring. In some embodiments, U is an optionally substituted 8- to 10-membered bicyclic aryl ring. In some embodiments, U is an optionally substituted 3- to 8-membered saturated or partially unsaturated monocyclic heterocyclic ring having 1-4 heteroatoms independently selected from nitrogen, oxygen, and sulfur. In some embodiments, U is an optionally substituted 5- to 6-membered monocyclic heteroaryl ring having 1-4 heteroatoms independently selected from nitrogen, oxygen, and sulfur. In some embodiments, U is an optionally substituted 6- to 10-membered bicyclic saturated or partially unsaturated heterocyclic ring having 1-5 heteroatoms independently selected from nitrogen, oxygen, and sulfur. In some embodiments, U is an optionally substituted 7- to 10-membered bicyclic heteroaryl ring having 1-5 heteroatoms independently selected from nitrogen, oxygen, and sulfur.

In some embodiments, U is —S(O)₂R. In some embodiments, U is —S(O)₂CH₃.

In some embodiments, U is an optionally substituted phenyl ring. In some embodiments, U is a phenyl ring, optionally substituted with halogen. In some embodiments, U is a phenyl ring, optionally substituted with fluorine. In some embodiments, U is a phenyl ring, optionally substituted with chlorine.

As defined above and described herein, two occurrences of U on adjacent carbon atoms can form an optionally substituted fused ring, selected from a fused phenyl ring; a fused 5- to 6-membered saturated or partially unsaturated heterocyclic ring containing 1-3 heteroatoms independently selected from nitrogen, oxygen, and sulfur; and a fused 5- to 6-membered heteroaryl ring containing 1-3 heteroatoms independently selected from nitrogen, oxygen, and sulfur.

In some embodiments, two occurrences of U on adjacent carbon atoms form a fused phenyl ring. In some embodiments, two occurrences of U on adjacent carbon atoms form an optionally substituted fused phenyl ring. In some embodiments, two occurrences of U on adjacent carbon atoms form a fused phenyl ring, optionally substituted with 1 or more halogen atoms. In some embodiments, two occurrences of U on adjacent carbon atoms form a fused phenyl ring, optionally substituted with one halogen atom. In some embodiments, two occurrences of U on adjacent carbon atoms form a fused phenyl ring, optionally substituted with fluorine. In some embodiments, two occurrences of U on adjacent carbon atoms form a fused phenyl ring, optionally substituted with chlorine. In some embodiments, two occurrences of U on adjacent carbon atoms form a fused phenyl ring, optionally substituted with 2 halogen atoms. In some embodiments, two occurrences of U on adjacent carbon atoms form a fused phenyl ring, optionally substituted with 2 fluorines. In some embodiments, two occurrences of U on adjacent carbon atoms form a fused phenyl ring, optionally substituted with 2 chlorines. In some embodiments, two occurrences of U on adjacent carbon atoms form a fused phenyl ring, optionally substituted with fluorine and chlorine.

In some embodiments, two occurrences of U on adjacent carbon atoms form a fused 5- to 6-membered heteroaryl ring containing 1-3 heteroatoms independently selected from nitrogen, oxygen, and sulfur. In some embodiments, two occurrences of U on adjacent carbon atoms form an optionally substituted fused 5- to 6-membered heteroaryl ring containing 1-3 heteroatoms independently selected from nitrogen, oxygen, and sulfur.

In some embodiments, two occurrences of U on adjacent carbon atoms form a fused 5-membered heteroaryl ring containing 1-3 heteroatoms independently selected from nitrogen, oxygen, and sulfur. In some embodiments, two occurrences of U on adjacent carbon atoms form an optionally substituted fused 5-membered heteroaryl ring containing 1-3 heteroatoms independently selected from nitrogen, oxygen, and sulfur.

In some embodiments, two occurrences of U on adjacent carbon atoms form a fused 5-membered heteroaryl ring containing one nitrogen and one oxygen heteroatom. In some embodiments, two occurrences of U on adjacent carbon atoms form an optionally substituted fused 5-membered heteroaryl ring containing one nitrogen and one oxygen heteroatom. In some embodiments, two occurrences of U on adjacent carbon atoms form a fused 5-membered heteroaryl ring containing one nitrogen and one oxygen heteroatom, optionally substituted with phenyl. In some embodiments, two occurrences of U on adjacent carbon atoms form a fused 5-membered heteroaryl ring containing one nitrogen and one oxygen heteroatom, optionally substituted with tosyl. In some embodiments, two occurrences of U on adjacent carbon atoms form a fused 5-membered heteroaryl ring containing one nitrogen and one oxygen heteroatom, optionally substituted with C₁₋₆ aliphatic. In some embodiments, two occurrences of U on adjacent carbon atoms form a fused 5-membered heteroaryl ring containing one nitrogen and one oxygen heteroatom, optionally substituted with C₁₋₆ alkyl. In some embodiments, two occurrences of U on adjacent carbon atoms form a fused 5-membered heteroaryl ring containing one nitrogen and one oxygen heteroatom, optionally substituted with cyclopropyl.

In some embodiments, two occurrences of U on adjacent carbon atoms form a fused 5-membered heteroaryl ring containing one nitrogen and one sulfur heteroatom. In some embodiments, two occurrences of U on adjacent carbon atoms form an optionally substituted fused 5-membered heteroaryl ring containing one nitrogen and one sulfur heteroatom. In some embodiments, two occurrences of U on adjacent carbon atoms form a fused 5-membered heteroaryl ring containing one nitrogen and one sulfur heteroatom, optionally substituted with phenyl.

In some embodiments, two occurrences of U on adjacent carbon atoms form a fused 5-membered heteroaryl ring containing two nitrogen heteroatoms. In some embodiments, two occurrences of U on adjacent carbon atoms form an optionally substituted fused 5-membered heteroaryl ring containing two nitrogen heteroatoms. In some embodiments, two occurrences of U on adjacent carbon atoms form a fused 5-membered heteroaryl ring containing two nitrogen heteroatoms, optionally substituted with phenyl.

In some embodiments, two occurrences of U on adjacent carbon atoms form a fused 6-membered heteroaryl ring containing 1-3 heteroatoms independently selected from nitrogen, oxygen, and sulfur. In some embodiments, two occurrences of U on adjacent carbon atoms form an optionally substituted fused 6-membered heteroaryl ring containing 1-3 heteroatoms independently selected from nitrogen, oxygen, and sulfur.

In some embodiments, two occurrences of U on adjacent carbon atoms form a fused 6-membered heteroaryl ring containing one nitrogen heteroatom. In some embodiments, two occurrences of U on adjacent carbon atoms form an optionally substituted fused 6-membered heteroaryl ring containing one nitrogen heteroatom. In some embodiments, two occurrences of U on adjacent carbon atoms form a fused 6-membered heteroaryl ring containing two nitrogen heteroatoms.

In some embodiments, two occurrences of U on adjacent carbon atoms form an optionally substituted fused 6-membered heteroaryl ring containing two nitrogen heteroatoms.

In some embodiments, the fused ring system formed by two occurrences of U on adjacent carbon atoms is quinazolinyl. In some embodiments, the fused ring system formed by two occurrences of U on adjacent carbon atoms is an optionally substituted quinazolinyl.

In some embodiments, the fused ring system formed by two occurrences of U on adjacent carbon atoms is quinolinyl. In some embodiments, the fused ring system formed by two occurrences of U on adjacent carbon atoms is optionally substituted quinolinyl. In some embodiments, the fused ring system formed by two occurrences of U on adjacent carbon atoms is quinolinyl, optionally substituted with 1-2 halogen atoms. In some embodiments, the fused ring system formed by two occurrences of U on adjacent carbon atoms is quinolinyl, optionally substituted with 1 halogen atom. In some embodiments, the fused ring system formed by two occurrences of U on adjacent carbon atoms is quinolinyl, optionally substituted with fluorine. In some embodiments, the fused ring system formed by two occurrences of U on adjacent carbon atoms quinolinyl, optionally substituted with chlorine.

In some embodiments, the fused ring system formed by two occurrences of U on adjacent carbon atoms is benzoxazolyl. In some embodiments, the fused ring system formed by two occurrences of U on adjacent carbon atoms is optionally substituted benzoxazolyl. In some embodiments, the fused ring system formed by two occurrences of U on adjacent carbon atoms is benzoxazolyl, optionally substituted with phenyl. In some embodiments, the fused ring system formed by two occurrences of U on adjacent carbon atoms is benzoxazolyl, optionally substituted with phenyl and a halogen atom. In some embodiments, the fused ring system formed by two occurrences of U on adjacent carbon atoms is benzoxazolyl, optionally substituted with phenyl and chlorine. In some embodiments, the fused ring system formed by two occurrences of U on adjacent carbon atoms is benzoxazolyl, optionally substituted with tosyl and chlorine.

In some embodiments, the fused ring system formed by two occurrences of U on adjacent carbon atoms is benzisoxazolyl. In some embodiments, the fused ring system formed by two occurrences of U on adjacent carbon atoms is optionally substituted benzisoxazolyl. In some embodiments, the fused ring system formed by two occurrences of U on adjacent carbon atoms is benzisoxazolyl, optionally substituted with phenyl. In some embodiments, the fused ring system formed by two occurrences of U on adjacent carbon atoms is benzisoxazolyl, optionally substituted with cyclopropyl and a halogen atom. In some embodiments, the fused ring system formed by two occurrences of U on adjacent carbon atoms is benzisoxazolyl, optionally substituted with cyclopropyl and chlorine.

In some embodiments, the fused ring system formed by two occurrences of U on adjacent carbon atoms is benzothiazolyl. In some embodiments, the fused ring system formed by two occurrences of U on adjacent carbon atoms is optionally substituted benzothiazolyl. In some embodiments, the fused ring system formed by two occurrences of U on adjacent carbon atoms is benzothiazolyl, optionally substituted with phenyl.

In some embodiments, the fused ring system formed by two occurrences of U on adjacent carbon atoms is benzisothiazolyl. In some embodiments, the fused ring system formed by two occurrences of U on adjacent carbon atoms is optionally substituted benzisothiazolyl. In some embodiments, the fused ring system formed by two occurrences of U on adjacent carbon atoms is benzisothiazolyl, optionally substituted with phenyl.

In some embodiments, the fused ring system formed by two occurrences of U on adjacent carbon atoms is benzimidazolyl. In some embodiments, the fused ring system formed by two occurrences of U on adjacent carbon atoms is optionally substituted benzimidazolyl. In some embodiments, the fused ring system formed by two occurrences of U on adjacent carbon atoms is benzimidazolyl, optionally substituted with phenyl.

In some embodiments, W, X, Y, and Z provide a phenyl ring. In some embodiments, W, X, Y, and Z provide a phenyl ring, substituted with k occurrences of U. In some embodiments where W, X, Y, and Z provide a phenyl ring, one of W, X, Y, and Z is —C—NH₂.

In some embodiments, W, X, Y, and Z provide a pyridinyl ring. In some embodiments, W, X, Y, and Z provide a pyridinyl ring, substituted with k occurrences of U. In some embodiments where W, X, Y, and Z provide a pyridinyl ring, one of W, X, Y, and Z is —C—NH₂.

In some embodiments, one of W, X, Y, and Z is —C—NH₂, one or more of the other of W, X, Y, and Z are CH; and k is 0. In some embodiments, one of W, X, and Y is —C—NH₂, one or more of the other of W, X, or Y are CH; Z is N; and k is 0.

In some embodiments, one of W, X, Y, and Z is —C—NH₂, one or more of the other of W, X, Y, and Z are CH; k is 1; and U is halogen. In some embodiments, one of W, X, Y, and Z is —C—NH₂, one or more of the other of W, X, Y, and Z are CH; k is 1; and U is fluorine. In some embodiments, one of W, X, Y, and Z is —C—NH₂, one or more of the other of W, X, Y, and Z are CH; k is 1; and U is chlorine. In some embodiments, one of X K, X, Y, and Z is —C—NH₂, one or more of the other of W, X, Y, and Z are CH; k is 1; and U is bromine.

In some embodiments, one of W, X, and Y is —C—NH₂, one or more of the other of W, X, and Y are CH; Z is N; k is 1; and U optionally substituted phenyl. In some embodiments, one of W, X, and Y is —C—NH₂; one or more of the other of W, X, and Y are CH; Z is N; k is 1; and U is phenyl, optionally substituted with halogen. In some embodiments, one of W, X, and Y is —C—NH₂, one or more of the other of W, X, and Y are CH; Z is N; k is 1; and U is phenyl, optionally substituted with chlorine. In some embodiments, one of W, X, and Y is —C—NH₂, one or more of the other of W, X, and Y are CH; Z is N; k is 1; and U is phenyl, optionally substituted with fluorine.

In some embodiments, W is N; one of X, Y, and Z is —C—NH₂; the other of X, Y, and Z are CH; k is 1; and U is optionally substituted phenyl. In some embodiments, W is N; one of X, Y, and Z is —C—NH₂; the other of X, Y, and Z are CH; k is 1; and U is phenyl, optionally substituted with halogen. In some embodiments, W is N; one of X, Y, and Z is —C—NH₂; the other of X, Y, and Z are CH; k is 1; and U is phenyl, optionally substituted with chlorine. In some embodiments, W is N; one of X, Y, and Z is —C—NH₂; the other of X, Y, and Z are CH; k is 1; and U is phenyl, optionally substituted with fluorine.

In some embodiments, one of W, X, and Y is —C—NH₂; one or more of the other of W, X, and Y are CH; Z is N; k is 2; and the two occurrences of U on adjacent carbon atoms form a fused phenyl ring. In some embodiments, one of W, X, and Y is —C—NH₂; one or more of the other of W, X, and Y are CH; Z is N; k is 2; and the two occurrences of U on adjacent carbon atoms form an optionally substituted fused phenyl ring. In some embodiments, one of W, X, and Y is —C—NH₂; one or more of the other of W, X, and Y are CH; Z is N; k is 2; and the two occurrences of U on adjacent carbon atoms form a fused phenyl ring, optionally substituted with halogen. In some embodiments, one of W, X, and Y is —C—NH₂; one or more of the other of W, X, and Y are CH; Z is N; k is 2; and the two occurrences of U on adjacent carbon atoms form a fused phenyl ring, optionally substituted with chlorine. In some embodiments, one of W, X, and Y is —C—NH₂; one or more of the other of W, X, and Y are CH; Z is N; k is 2; and the two occurrences of U on adjacent carbon atoms form a fused phenyl ring, optionally substituted with fluorine.

In some embodiments, W is N; one of X, Y, and Z is —C—NH₂; the other of X, Y, and Z are CH; k is 2; and the two occurrences of U on adjacent carbon atoms form a fused phenyl ring. In some embodiments, W is N; one of X, Y, and Z is —C—NH₂; the other of X, Y, and Z are CH; k is 2; and the two occurrences of U on adjacent carbon atoms form an optionally substituted fused phenyl ring. In some embodiments, W is N; one of X, Y, and Z is —C—NH₂; the other of X, Y, and Z are CH; k is 2; and the two occurrences of U on adjacent carbon atoms form a fused phenyl ring, optionally substituted with halogen. In some embodiments, W is N; one of X, Y, and Z is —C—NH₂; the other of X, Y, and Z are CH; k is 2; and the two occurrences of U on adjacent carbon atoms form a fused phenyl ring, optionally substituted with fluorine. In some embodiments, W is N; one of X, Y, and Z is —C—NH₂; the other of X, Y, and Z are CH; k is 2; and the two occurrences of U on adjacent carbon atoms form a fused phenyl ring, optionally substituted with chlorine. In some embodiments, W is N; one of X, Y, and Z is —C—NH₂; the other of X, Y, and Z are CH; k is 2; and the two occurrences of U on adjacent carbon atoms form a fused phenyl ring, optionally substituted with chlorine and fluorine. In some embodiments, W is N; one of X, Y, and Z is —C—NH₂; the other of X, Y, and Z are CH; k is 2; and the two occurrences of U on adjacent carbon atoms form a fused phenyl ring, optionally substituted with chlorine at 2 positions.

In some embodiments, one of W, X, Y, and Z is —C—NH₂; one or more of the other of W, X, Y, and Z are CH; k is 2; and the two occurrences of U on adjacent carbon atoms form a fused 5- to 6-membered heteroaryl ring containing 1-3 heteroatoms independently selected from nitrogen, oxygen, and sulfur. In some embodiments, one of W, X, Y, and Z is —C—NH₂; one or more of the other of W, X, Y, and Z are CH; k is 2; and the two occurrences of U on adjacent carbon atoms form an optionally substituted fused 5- to 6-membered heteroaryl ring containing 1-3 heteroatoms independently selected from nitrogen, oxygen, and sulfur.

In some embodiments, one of W, X, Y, and Z is —C—NH₂; one or more of the other of W, X, Y, and Z are CH; k is 2; and the two occurrences of U on adjacent carbon atoms form an optionally substituted fused 6-membered heteroaryl ring containing one nitrogen heteroatom. In some embodiments, one of W, X, Y, and Z is —C—NH₂; one or more of the other of W, X, Y, and Z are CH; k is 2; and the two occurrences of U on adjacent carbon atoms form a fused pyridine ring. In some embodiments, one of W, X, Y, and Z is —C—NH₂; one or more of the other of W, X, Y, and Z are CH; k is 2; and the two occurrences of U on adjacent carbon atoms form an optionally substituted fused pyridine ring. In some embodiments, one of W, X, Y, and Z is —C—NH₂; one or more of the other of W, X, Y, and Z are CH; k is 2; and the two occurrences of U on adjacent carbon atoms form an optionally substituted fused 6-membered heteroaryl ring containing two nitrogen heteroatoms. In some embodiments, one of W, X, Y, and Z is —C—NH₂; one or more of the other of W, X, Y, and Z are CH; k is 2; and the two occurrences of U on adjacent carbon atoms form a fused pyrimidine ring. In some embodiments, one of W, X, Y, and Z is —C—NH₂; one or more of the other of W, X, Y, and Z are CH; k is 2; and the two occurrences of U on adjacent carbon atoms form an optionally substituted fused pyrimidine ring.

In some embodiments, one of W, X, Y, and Z is —C—NH₂; one or more of the other of W, X, Y, and Z are CH; k is 2; and the two occurrences of U on adjacent carbon atoms form fused aryl ring with 2 heteroatoms. In some embodiments, one of W, X, Y, and Z is —C—NH₂; one or more of the other of W, X, Y, and Z are CH; k is 2; and the two occurrences of U on adjacent carbon atoms form a 5-membered fused oxazole ring. In some embodiments, one of W, X, Y, and Z is —C—NH₂; one or more of the other of W, X, Y, and Z are CH; k is 2; and the two occurrences of U on adjacent carbon atoms form a 5-membered fused oxazole ring, optionally substituted with phenyl.

In some embodiments, one of W, X, Y, and Z is —C—NH₂; one or more of the other of W, X, Y, and Z is CH; k is 2; and the two occurrences of U on adjacent carbon atoms form an optionally substituted fused 5-membered heteroaryl ring containing one nitrogen and one oxygen heteroatom. In some embodiments, one of W, K Y, and Z is —C—NH₂; one or more of the other of W, K, Y, and Z is CH; k is 2; and the two occurrences of U on adjacent carbon atoms form a fused 5-membered heteroaryl ring containing one nitrogen and one oxygen heteroatoms, optionally substituted with phenyl. In some embodiments, one of W, X, Y, and Z is —C—NH₂; one or more of the other of W, X, Y, and Z is CH; k is 2; and the two occurrences of U on adjacent carbon atoms form a fused 5-membered heteroaryl ring containing one nitrogen and one oxygen heteroatoms, optionally substituted with tosyl. In some embodiments, one of W, X, Y, and Z is —C—NH₂; one or more of the other of W, X, Y, and Z is CH; k is 2; and the two occurrences of U on adjacent carbon atoms form a fused 5-membered heteroaryl ring containing one nitrogen and one oxygen heteroatoms, optionally substituted with cyclopropyl.

In some embodiments, one of W, X, Y, and Z is —C—NH₂; one or more of the other of W, X, Y, and Z are CH; k is 2; and the two occurrences of U on adjacent carbon atoms form an optionally substituted fused oxazole ring. In some embodiments, one of W, X, Y, and Z is —C—NH₂; one or more of the other of W, X, Y, and Z are CH; k is 2; and the two occurrences of U on adjacent carbon atoms form a fused oxazole ring, optionally substituted with phenyl. In some embodiments, one of W, X, Y, and Z is —C—NH₂; one or more of the other of W, X, Y, and Z are CH; k is 2; and the two occurrences of U on adjacent carbon atoms form a fused oxazole ring, optionally substituted with tosyl.

In some embodiments, one of W, X, Y, and Z is —C—NH₂; one or more of the other of W, X, Y, and Z are CH; k is 2; and the two occurrences of U on adjacent carbon atoms form an optionally substituted fused isoxazole ring. In some embodiments, one of W, X, Y, and Z is —C—NH₂; one or more of the other of W, X, Y, and Z are CH; k is 2; and the two occurrences of U on adjacent carbon atoms form a fused isoxazole ring, optionally substituted with phenyl. In some embodiments, one of W, X, Y, and Z is —C—NH₂; one or more of the other of W, X, Y, and Z are CH; k is 2; and the two occurrences of U on adjacent carbon atoms form a fused isoxazole ring, optionally substituted with cyclopropyl.

In some embodiments, one of W, X, Y, and Z is —C—NH₂; one or more of the other of W, X, Y, and Z are CH; k is 2; and the two occurrences of U on adjacent carbon atoms form an optionally substituted fused 5-membered heteroaryl ring containing one nitrogen and one sulfur heteroatom. In some embodiments, one of W, X, Y, and Z is —C—NH₂; one or more of the other of W, X, Y, and Z is CH; k is 2; and the two occurrences of U on adjacent carbon atoms form a fused 5-membered heteroaryl ring containing one nitrogen and one sulfur heteroatom, optionally substituted by phenyl.

In some embodiments, one of W, X, Y, and Z is —C—NH₂; one or more of the other of W, X, Y, and Z are CH; k is 2; and the two occurrences of U on adjacent carbon atoms form an optionally substituted fused thiazole ring. In some embodiments, one of W, X, Y, and Z is —C—NH₂; one or more of the other of W, X, Y, and Z are CH; k is 2; and the two occurrences of U on adjacent carbon atoms form a fused thiazole ring, optionally substituted with phenyl.

In some embodiments, one of W, X, Y, and Z is —C—NH₂; one or more of the other of W, X, Y, and Z are CH; k is 2; and the two occurrences of U on adjacent carbon atoms form an optionally substituted fused 5 membered heteroaryl ring containing two nitrogen heteroatoms. In some embodiments, one of W, X, Y, and Z is —C—NH₂; one or more of the other of W, X, Y, and Z are CH; k is 2; and the two occurrences of U on adjacent carbon atoms form an optionally substituted fused imidazole ring. In some embodiments, one of W, X, Y, and Z is —C—NH₂; one or more of the other of W, X, Y, and Z are CH; k is 2; and the two occurrences of U on adjacent carbon atoms form a fused imidazole ring, optionally substituted with phenyl.

In some embodiments, one of W, X, Y, and Z is —C—NH₂; one or more of the other of W, X, Y, and Z are CH; k is 3; U₁ is chlorine and U₂ and U₃ on adjacent carbon atoms form an optionally substituted fused 5-membered heteroaryl ring containing one nitrogen and one oxygen heteroatom. In some embodiments, one of W, X, Y, and Z is —C—NH₂; one or more of the other of W, X, Y, and Z are CH; k is 3; U₁ is chlorine and U₂ and U₃ on adjacent carbon atoms form a fused 5-membered heteroaryl ring containing one nitrogen and one oxygen heteroatom, optionally substituted with phenyl. In some embodiments, one of W, X, Y, and Z is —C—NH₂; one or more of the other of W, X, Y, and Z are CH; k is 3; U₁ is chlorine and U₂ and U₃ on adjacent carbon atoms form a fused 5-membered heteroaryl ring containing one nitrogen and one oxygen heteroatom, optionally substituted with tosyl.

In some embodiments, one of W, X, Y, and Z is —C—NH₂; one or more of the other of W, X, Y, and Z are CH; k is 3; U₁ is chlorine and U₂ and U₃ on adjacent carbon atoms form an optionally substituted fused oxazole ring. In some embodiments, one of W, X, Y, and Z is —C—NH₂; one or more of the other of W, X, Y, and Z are CH; k is 3; U₁ is chlorine and U₂ and U₃ on adjacent carbon atoms form a fused oxazole ring, optionally substituted with phenyl. In some embodiments, one of W, X, Y, and Z is —C—NH₂; one or more of the other of W, X, Y, and Z are CH; k is 3; U₁ is chlorine and U₂ and U₃ on adjacent carbon atoms form a fused oxazole ring, optionally substituted with tosyl.

In some embodiments, one of W, X, Y, and Z is —C—NH₂; one or more of the other of W, X, Y, and Z are CH; k is 3; U₁ is chlorine and U₂ and U₃ on adjacent carbon atoms form an optionally substituted fused isoxazole ring. In some embodiments, one of W, X, Y, and Z is —C—NH₂; one or more of the other of W, X, Y, and Z are CH; k is 3; U₁ is chlorine and U₂ and U₃ adjacent carbon atoms form a fused isoxazole ring, optionally substituted with cyclopropyl.

As defined above and described herein, each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In some embodiments, R is hydrogen. In some embodiments, R is deuterium. In some embodiments, R is C₁₋₆ aliphatic. In some embodiments R is methyl. In some embodiments, R is ethyl. In some embodiments, R is optionally substituted C₁₋₆ aliphatic. In some embodiments, R is optionally substituted methyl. In some embodiments, R is optionally substituted ethyl. In some embodiments, R is phenyl. In some embodiments, R is optionally substituted phenyl. In some embodiments, R is phenyl, optionally substituted with halogen. In some embodiments, R is phenyl, optionally substituted with fluorine.

As described generally above, R^{a} is C₁₋₄ aliphatic optionally substituted with 1, 2, or 3 deuterium or halogen atoms.

In some embodiments, R^{a} is C₁₋₄ aliphatic. In some embodiments, R^{a} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments, R^{a} is C₁₋₄ alkyl. In some embodiments, R^{a} is C₁₋₄ alkyl optionally substituted with 1, 2, or 3 deuterium or halogen atoms. In some embodiments, R is C₁₋₄ alkyl optionally substituted with 1, 2, or 3 halogen atoms. In some embodiments, R^{a} is methyl or ethyl optionally substituted with 1, 2, or 3 halogen atoms. In some embodiments, R^{a} is methyl.

As defined generally above, R^{b} is C₁₋₄ aliphatic optionally substituted with 1, 2, or 3 deuterium or halogen atoms.

In some embodiments, R^{b} is C₁₋₄ aliphatic. In some embodiments, R^{b} is C₁₋₄ aliphatic optionally substituted with 1, 2, or 3 deuterium atoms. In some embodiments, R^{b} is C₁₋₄ aliphatic optionally substituted with 1, 2, or 3 halogen atoms.

In some embodiments, R is C₁₋₄ alkyl. In some embodiments, R^{b} is C₁₋₄ alkyl optionally substituted with 1, 2, or 3 deuterium or halogen atoms. In some embodiments, R^{b} is C₁₋₄ alkyl optionally substituted with 1, 2, or 3 halogen atoms. In some embodiments, R^{b} is methyl or ethyl optionally substituted with 1, 2, or 3 halogen atoms. In some embodiments, R^{b} is methyl.

As defined generally above, in some embodiments, R^{a} and R^{b}, taken together with the carbon atom to which they are attached, form a 3- to 8-membered cycloalkyl or heterocyclyl ring containing 1-2 heteroatoms selected from nitrogen, oxygen, and sulfur.

In some embodiments, R^{a} and R^{b}, taken together with the carbon atom to which they are attached, form a 3- to 8-membered cycloalkyl ring. In some embodiments, R^{a} and R^{b}, taken together with the carbon atom to which they are attached, form a 3- to 8-membered heterocyclyl ring containing 1-2 heteroatoms selected from nitrogen, oxygen, and sulfur.

In some embodiments, R^{a} and R^{b}, taken together with the carbon atom to which they are attached, form a cyclopropyl, cyclobutyl, or cyclopentyl ring. In some embodiments, R^{a} and R^{b}, taken together with the carbon atom to which they are attached, form an oxirane, oxetane, tetrahydrofuran, or aziridine.

In some embodiments, R^{a} and R^{b} are methyl.

In some embodiments, the pharmaceutical composition comprises a compound of formula II:

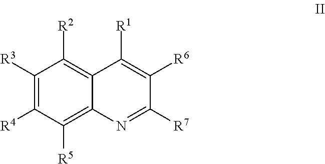

or a pharmaceutically acceptable salt thereof, wherein:

each of R¹, R⁷, and R⁸ is independently H, D, halogen, —NH₂, —CN, —OR, —SR, optionally substituted C₁₋₆ aliphatic, or

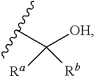

wherein one of R¹, R⁷ and R⁸ is —NH₂ and other one of R¹ R⁷ and R⁸ is

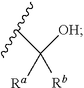

R² is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R), —C(O)R, —C(O)OR, —OC(O)R, —S(O)R, and —S(O)₂R;

R³ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;

R⁴ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;

R^{a} is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;

R^{a} is C₁₋₄ aliphatic optionally substituted with 1, 2, or 3 deuterium or halogen atoms;

R^{b} is C₁₋₄ aliphatic optionally substituted with 1, 2, or 3 deuterium or halogen atoms; or R^{a} and R^{b}, taken together with the carbon atom to which they are attached, form a 3- to 8-membered cycloalkyl or heterocyclyl ring containing 1-2 heteroatoms selected from nitrogen, oxygen, and sulfur; and

each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In some embodiments, the —NH₂ and —CR^{a}R^{b}OH groups are on adjacent carbon atoms of the compound of formula II.

In some embodiments of formula II, R^{a} is C₁₋₄ aliphatic. In some embodiments, R^{a} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments of formula II, R^{a} is C₁₋₄ alkyl. In some embodiments, R^{a} is C₁₋₄ alkyl optionally substituted with 1, 2, or 3 deuterium or halogen atoms. In some embodiments, R^{a} is C₁₋₄ alkyl optionally substituted with 1, 2, or 3 halogen atoms. In some embodiments, R^{a} is methyl or ethyl optionally substituted with 1, 2, or 3 halogen atoms. In some embodiments, R^{a} is methyl.

As defined generally above, R^{b} is C₁₋₄ aliphatic optionally substituted with 1, 2, or 3 deuterium or halogen atoms.

In some embodiments of formula II, R^{b} is C₁₋₄ aliphatic. In some embodiments, R^{b} is C₁₋₄ aliphatic optionally substituted with 1, 2, or 3 deuterium atoms. In some embodiments, R^{b} is C₁₋₄ aliphatic optionally substituted with 1, 2, or 3 halogen atoms.

In some embodiments of formula II, R^{b} is C₁₋₄ alkyl. In some embodiments, R^{b} is C₁₋₄ alkyl optionally substituted with 1, 2, or 3 deuterium or halogen atoms. In some embodiments, R^{b} is C₁₋₄ alkyl optionally substituted with 1, 2, or 3 halogen atoms. In some embodiments, R^{b} is methyl or ethyl optionally substituted with 1, 2, or 3 halogen atoms. In some embodiments, R^{b} is methyl.

As defined generally above, in some embodiments, R^{a} and R^{b}, taken together with the carbon atom to which they are attached, form a 3- to 8-membered cycloalkyl or heterocyclyl ring containing 1-2 heteroatoms selected from nitrogen, oxygen, and sulfur.

In some embodiments of formula II, R^{a} and R^{b}, taken together with the carbon atom to which they are attached, form a 3- to 8-membered cycloalkyl ring. In some embodiments, R^{a} and R^{b}, taken together with the carbon atom to which they are attached, form a 3- to 8-membered heterocyclyl ring containing 1-2 heteroatoms selected from nitrogen, oxygen, and sulfur.

In some embodiments of formula II, R^{a} and R^{b}, taken together with the carbon atom to which they are attached, form a cyclopropyl, cyclobutyl, or cyclopentyl ring. In some embodiments, R^{a} and R^{b}, taken together with the carbon atom to which they are attached, form an oxirane, oxetane, tetrahydrofuran, or aziridine.

In some embodiments of formula II, the —NH₂ on one of R¹, R⁷, and R⁸ and the carbinol on the other of R¹, R⁷, and R⁸ are on adjacent carbon atoms of the pyridine moiety.

In some embodiments, the compound is a compound of formula II-a, II-b, or II-c:

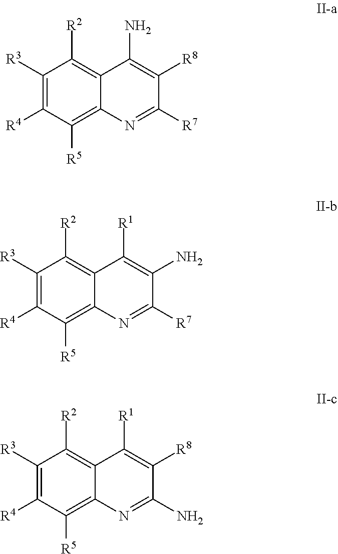

or a pharmaceutically acceptable salt thereof, wherein:

each of R¹, R⁷, and R⁸ when present is independently H, D, halogen, —CN, —OR, —SR, optionally substituted C₁₋₆ aliphatic, or

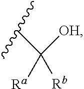

wherein one of R¹, R⁷, and R⁸ is

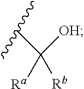

and

R², R³, R⁴, R⁵, R^{a}, R^{b} and R are as defined for formula II.

In some embodiments, the pharmaceutical composition comprises a compound of formula II-d, II-e, II-f or II-g:

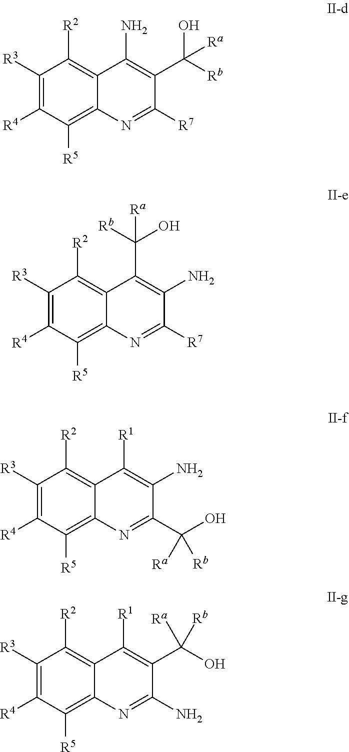

or a pharmaceutically acceptable salt thereof, wherein;

R¹ and R⁷ is independently H, D, halogen, —CN, —OR, —SR, optionally substituted C₁₋₆ aliphatic; and

R², R³, R⁴, R⁵, R^{a}, R^{b} and R are as defined for formula II.

In some embodiments, the pharmaceutical composition comprises a compound of formula III:

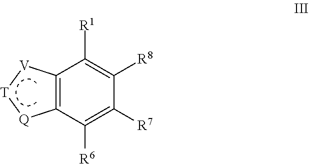

or a pharmaceutically acceptable salt thereof, wherein:

Q, T and V are independently S, N, O, or —C—R;

each of R¹, R⁶, R⁷, and R⁸ is independently H, D, halogen, —NH₂, —CN, —OR, —SR, optionally substituted C₁₋₆ aliphatic, or

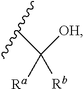

wherein one of R¹, R⁶, R⁷, and R⁸ is —NH₂ and other one of R¹, R⁶, R⁷, and R⁸ is

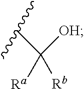

R^{a} is C₁₋₄ aliphatic optionally substituted with 1, 2, or 3 deuterium or halogen atoms; and

R^{b} is C₁₋₄ aliphatic optionally substituted with 1, 2, or 3 deuterium or halogen atoms; or R^{a} and R^{b}, taken together with the carbon atom to which they are attached, form a 3-8 membered cycloalkyl or heterocyclyl ring containing 1-2 heteroatoms selected from nitrogen, oxygen, and sulfur; and

each R is independently selected from hydrogen, deuterium, and an optionally substituted group selected from: C₁₋₆ aliphatic, a 3- to 8-membered saturated or partially unsaturated monocyclic carbocyclic ring, phenyl, an 8- to 10-membered bicyclic aryl ring, a 3- to 8-membered saturated or partially unsaturated monocyclic heterocyclic ring having 1-4 heteroatoms independently selected from nitrogen, oxygen, and sulfur, a 5- to 6-membered monocyclic heteroaryl ring having 1-4 heteroatoms independently selected from nitrogen, oxygen, and sulfur, a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In some embodiments of formula III, the —NH₂ on one of R¹, R⁶, R⁷, and R⁸ and the carbinol on the other of R¹, R⁶, R⁷, and R⁸ are on adjacent carbon atoms of the phenyl moiety.

In some embodiments of formula III, one of Q, T and V is N, and other of Q, T and V is O. In some embodiments, Q is O, V is N, and T is C—R. In some embodiments, Q is N, T is O and V is C—R.

In some embodiments of formula III, the compound is a compound of formula III-a or III-b:

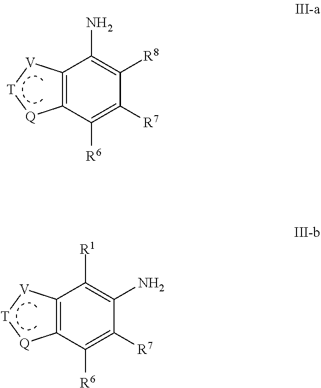

or a pharmaceutically acceptable salt thereof, wherein:

each of R¹, R⁶, R⁷, and R⁸ when present is independently H, D, halogen, —CN, —OR, —SR, optionally substituted C₁₋₆ aliphatic, or

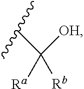

wherein one of R¹, R⁶, R⁷, and R⁸ is

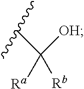

and

Q, T, V, R, R^{a} and R^{b} are as defined in formula III.

In some embodiments of formula III, the compound is a compound of formula III-c, III-d or III-e:

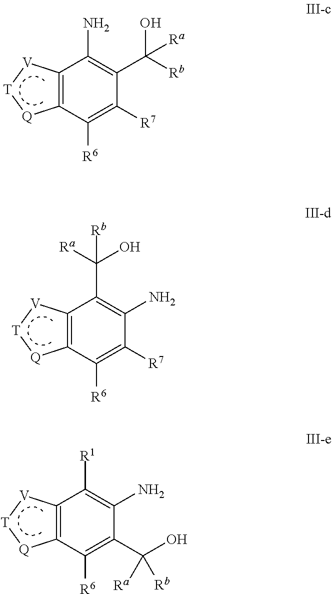

or a pharmaceutically acceptable salt thereof, wherein:

each of R¹, R⁶, and R⁷ when present is independently H, D, halogen, —CN, —OR, —SR, optionally substituted C₁₋₆ aliphatic; and

Q, T, V, R, R^{a} and R^{b} are as defined in formula III.

In some embodiments of formula III, the compound is a compound of formula III-f, III-g, III-h or III-i:

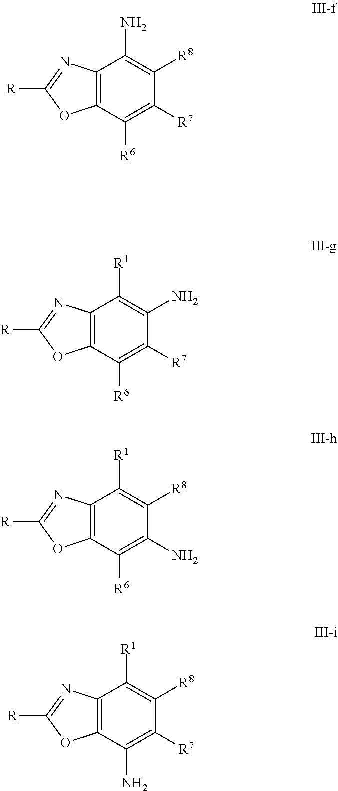

or a pharmaceutically acceptable salt thereof, wherein:

each of R¹, R⁶, R⁷, and R⁸ when present is independently H, D, halogen, —CN, —OR, —SR, optionally substituted C₁₋₆ aliphatic or

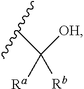

wherein one of R¹, R⁶, R⁷, and R⁸ is

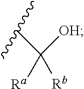

and

R, R^{a} and R^{b} are as defined in formula III.

In some embodiments of formula III, the compound is a compound of formula III-j, III-k, III-l, III-l′, III-m or III-m′:

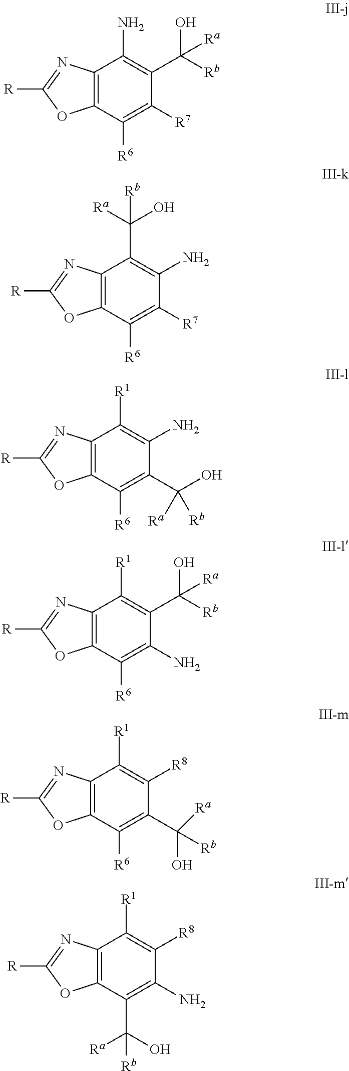

or a pharmaceutically acceptable salt thereof, wherein:

each of R¹, R⁶, R⁷, and R⁸ when present is independently H, D, halogen, —CN, —OR, —SR, optionally substituted C₁₋₆ aliphatic; and

R, R^{a} and R^{b} are as defined in formula III.

In some embodiments of formula III, the compound is a compound of formula III-n:

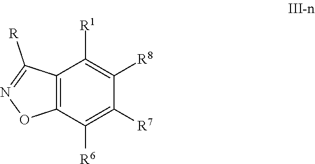

or a pharmaceutically acceptable salt thereof, wherein:

each of R¹, R⁶, R⁷, and R⁸ is independently H, D, halogen, —NH₂, —CN, —OR, —SR, optionally substituted C₁₋₆ aliphatic, or

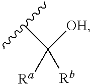

wherein one of R¹, R⁶, R⁷, and R⁸ is —NH₂ and other one of R¹, R⁶, R⁷, and R⁸ is

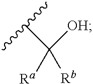

and

R, R^{a}, and R^{b} are as defined in formula III.

In some embodiments of formula III, the compound is a compound of formula III-o, III-p, III-q or III-r:

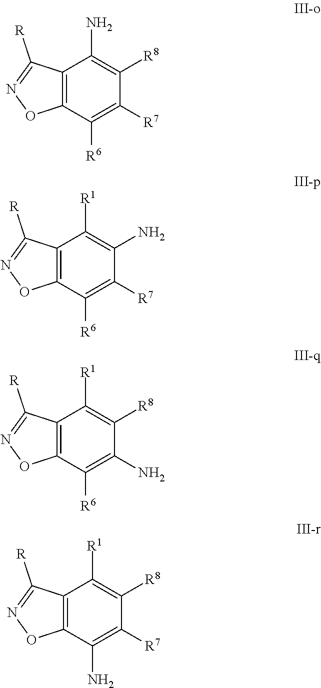

or a pharmaceutically acceptable salt thereof, wherein:
each of R¹, R⁶, R⁷, and R⁸ when present is independently H, D, halogen, —CN, —OR, —SR, optionally substituted C₁₋₆ aliphatic, or

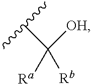

wherein one of R¹, R⁶, R⁷, and R⁸ is

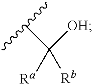

and

R, R^{a}, and R^{b} are as defined in formula III.

In some embodiments of formula III, the compound is a compound of formula III-s, III-t, III-u, III-v, III-w, or III-x:

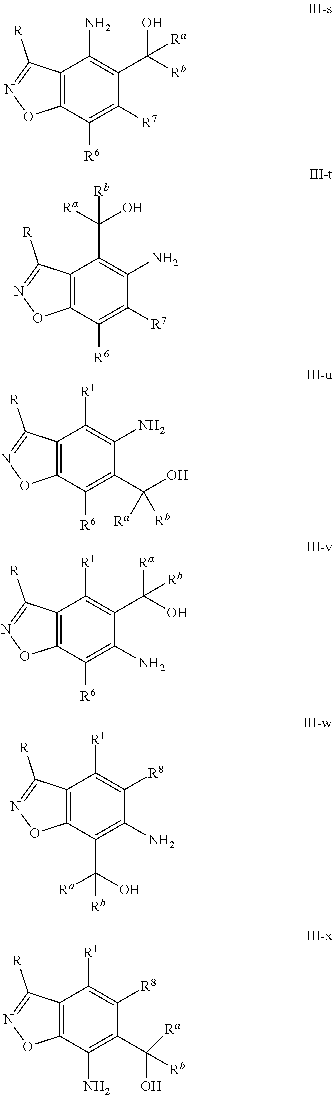

or a pharmaceutically acceptable salt thereof, wherein:

each of R¹, R⁶, R⁷, and R⁸ when present is independently H, D, halogen, —CN, —OR, —SR, optionally substituted C₁₋₆ aliphatic; and

R, R^{a} and R^{b} are as defined in formula III.

In some embodiments, the pharmaceutical composition comprises a compound of formula IV:

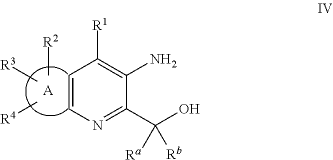

or a pharmaceutically acceptable salt thereof, wherein:
Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom; or a 6-membered partially unsaturated heterocyclic or heteroaromatic ring containing 1-3 heteroatoms independently selected from nitrogen, oxygen, and sulfur; or a 7-membered partially unsaturated heterocyclic or heteroaromatic ring containing 1-3 heteroatoms independently selected from nitrogen, oxygen, and sulfur;
R¹ is H, D, halogen, —CN, —OR, —SR, or optionally substituted C₁₋₆ aliphatic;
R² is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is absent or is selected from —R, halogen, —CN, —OR, —SR, —N(R)₂, —N(R)C(O)R, —C(O)N(R), —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{a} is C₁₋₄ aliphatic optionally substituted with 1, 2, or 3 deuterium or halogen atoms;
R^{b} is C₁₋₄ aliphatic optionally substituted with 1, 2, or 3 deuterium or halogen atoms; or
R^{a} and R^{b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from: C₁₋₆ aliphatic, a 3- to 8-membered saturated or partially unsaturated monocyclic carbocyclic ring, phenyl, an 8- to 10-membered bicyclic aryl ring, a 3- to 8-membered saturated or partially unsaturated monocyclic heterocyclic ring having 1-4 heteroatoms independently selected from nitrogen, oxygen, and sulfur, a 5- to 6-membered monocyclic heteroaryl ring having 1-4 heteroatoms independently selected from nitrogen, oxygen, and sulfur, a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

As defined generally above, Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom; or a 6-membered partially unsaturated heterocyclic or heteroaromatic ring containing 1-3 heteroatoms independently selected from nitrogen, oxygen, and sulfur; or a 7-membered partially unsaturated heterocyclic or heteroaromatic ring containing 1-3 heteroatoms independently selected from nitrogen, oxygen, and sulfur.

In some embodiments, Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom. In some embodiments, Ring A is a 6-membered partially unsaturated heterocyclic or heteroaromatic ring containing 1-3 heteroatoms independently selected from nitrogen, oxygen, and sulfur. In some embodiments, Ring A is a 7-membered partially unsaturated heterocyclic or heteroaromatic ring containing 1-3 heteroatoms independently selected from nitrogen, oxygen, and sulfur.

In some embodiments, Ring A is imidazole or triazole. In some embodiments, Ring A is thiazole. In some embodiments, Ring A is thiophene or furan. In some embodiments, Ring A is pyridine, pyrimidine, pyrazine, pyridazine, or 1,2,4-triazine. In some embodiments, Ring A is pyridine.

As defined generally above, R¹ is H, D, halogen, —CN, —OR, —SR, or optionally substituted C₁₋₆ aliphatic.

In some embodiments, R¹ is H. In some embodiments, R¹ is D. In some embodiments, R¹ is halogen. In some embodiments, R¹ is —CN. In some embodiments, R¹ is —OR. In some embodiments, R¹ is —SR. In some embodiments, R¹ is optionally substituted C₁₋₆ aliphatic.

As described generally above, R² is absent or is selected from —R, halogen, —CN, —OR, —SR, N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R² is absent. In some embodiments, R² is —R. In some embodiments. R² is halogen. In some embodiments, R² is —CN. In some embodiments, R² is —OR. In some embodiments, R² is —SR. In some embodiments, R² is —N(R)₂. In some embodiments, R² is —N(R)C(O)R. In some embodiments, R² is —C(O)N(R)₂. In some embodiments, R² is —N(R)C(O)N(R)₂. In some embodiments, R² is —N(R)C(O)OR. In some embodiments, R² is —OC(O)N(R)₂. In some embodiments, R² is —N(R)S(O)₂R. In some embodiments, R² is —SO₂N(R)₂. In some embodiments, R² is —C(O)R. In some embodiments, R² is —C(O)OR. In some embodiments, R² is —OC(O)R. In some embodiments, R² is —S(O)R. In some embodiments, R² is —S(O)₂R.

In some embodiments, R² is hydrogen. In some embodiments, R² is deuterium. In some embodiments, R² is an optionally substituted C₁₋₆ aliphatic. In some embodiments, R² is an optionally substituted 3- to 8-membered saturated or partially unsaturated monocyclic carbocyclic ring. In some embodiments, R² is an optionally substituted phenyl. In some embodiments, R² is an optionally substituted 8- to 10-membered bicyclic aryl ring. In some embodiments, R² is an optionally substituted 3- to 8-membered saturated or partially unsaturated monocyclic heterocyclic ring having 1-4 heteroatoms independently selected from nitrogen, oxygen, and sulfur. In some embodiments, R² is an optionally substituted 5- to 6-membered monocyclic heteroaryl ring having 1-4 heteroatoms independently selected from nitrogen, oxygen, and sulfur. In some embodiments, R² is an optionally substituted 6- to 10-membered bicyclic saturated or partially unsaturated heterocyclic ring having 1-5 heteroatoms independently selected from nitrogen, oxygen, and sulfur. In some embodiments, R² is an optionally substituted 7- to 10-membered bicyclic heteroaryl ring having 1-5 heteroatoms independently selected from nitrogen, oxygen, and sulfur.

In some embodiments, R² is Cl or Br. In some embodiments, R² is Cl.

As defined generally above, R³ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R³ is absent. In some embodiments, R³ is —R. In some embodiments, R³ is halogen. In some embodiments, R³ is —CN. In some embodiments, R³ is —OR. In some embodiments, R³ is —SR. In some embodiments, R³ is —N(R)₂. In some embodiments, R³ is —N(R)C(O)R. In some embodiments, R³ is —C(O)N(R)₂. In some embodiments, R³ is —N(R)C(O)N(R)₂. In some embodiments, R³ is —N(R)C(O)OR. In some embodiments, R³ is —OC(O)N(R)₂. In some embodiments, R³ is —N(R)S(O)₂R. In some embodiments, R³ is —SO₂N(R)₂. In some embodiments, R³ is —C(O)R. In some embodiments, R³ is —C(O)OR. In some embodiments, R³ is —OC(O)R. In some embodiments, R³ is —S(O)R. In some embodiments, R³ is —S(O)₂R.

In some embodiments, R³ is hydrogen. In some embodiments, R³ is deuterium. In some embodiments, R³ is an optionally substituted C₁₋₄ aliphatic. In some embodiments, R³ is an optionally substituted 3- to 8-membered saturated or partially unsaturated monocyclic carbocyclic ring. In some embodiments, R³ is an optionally substituted phenyl. In some embodiments, R³ is an optionally substituted 8- to 10-membered bicyclic aryl ring. In some embodiments, R³ is an optionally substituted 3- to 8-membered saturated or partially unsaturated monocyclic heterocyclic ring having 1-4 heteroatoms independently selected from nitrogen, oxygen, and sulfur. In some embodiments, R³ is an optionally substituted 5- to 6-membered monocyclic heteroaryl ring having 1-4 heteroatoms independently selected from nitrogen, oxygen, and sulfur. In some embodiments, R³ is an optionally substituted 6- to 10-membered bicyclic saturated or partially unsaturated heterocyclic ring having 1-5 heteroatoms independently selected from nitrogen, oxygen, and sulfur. In some embodiments, R³ is an optionally substituted 7- to 10-membered bicyclic heteroaryl ring having 1-5 heteroatoms independently selected from nitrogen, oxygen, and sulfur.

In some embodiments, R³ is Cl or Br. In some embodiments, R³ is Cl.

As defined generally above, R⁴ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R⁴ is absent. In some embodiments, R⁴ is —R. In some embodiments, R⁴ is halogen. In some embodiments, R⁴ is —CN. In some embodiments, R⁴ is —OR. In some embodiments, R⁴ is —SR. In some embodiments, R⁴ is —N(R)₂. In some embodiments, R⁴ is —N(R)C(O)R. In some embodiments, R⁴ is —C(O)N(R)₂. In some embodiments, R⁴ is —N(R)C(O)N(R)₂. In some embodiments, R⁴ is —N(R)C(O)OR. In some embodiments, R⁴ is —OC(O)N(R)₂. In some embodiments, R⁴ is —N(R)S(O)₂R. In some embodiments, R⁴ is —SO₂N(R)₂. In some embodiments, R⁴ is —C(O)R. In some embodiments, R⁴ is —C(O)OR. In some embodiments, R⁴ is —OC(O)R. In some embodiments, R⁴ is —S(O)R. In some embodiments, R⁴ is —S(O)₂R.

In some embodiments, R⁴ is hydrogen. In some embodiments, R⁴ is deuterium. In some embodiments, R⁴ is an optionally substituted C₁₋₆ aliphatic. In some embodiments, R⁴ is an optionally substituted 3- to 8-membered saturated or partially unsaturated monocyclic carbocyclic ring. In some embodiments, R⁴ is an optionally substituted phenyl. In some embodiments, R⁴ is an optionally substituted 8- to 10-membered bicyclic aryl ring. In some embodiments, R⁴ is an optionally substituted 3- to 8-membered saturated or partially unsaturated monocyclic heterocyclic ring having 1-4 heteroatoms independently selected from nitrogen, oxygen, and sulfur. In some embodiments, R⁴ is an optionally substituted 5- to 6-membered monocyclic heteroaryl ring having 1-4 heteroatoms independently selected from nitrogen, oxygen, and sulfur. In some embodiments, R⁴ is an optionally substituted 6- to 10-membered bicyclic saturated or partially unsaturated heterocyclic ring having 1-5 heteroatoms independently selected from nitrogen, oxygen, and sulfur. In some embodiments, R⁴ is an optionally substituted 7- to 10-membered bicyclic heteroaryl ring having 1-5 heteroatoms independently selected from nitrogen, oxygen, and sulfur.

In some embodiments, R⁴ is Cl or Br. In some embodiments, R⁴ is Cl.

As described generally above, R^{a} is C₁₋₄ aliphatic optionally substituted with 1, 2, or 3 deuterium or halogen atoms.

In some embodiments, R^{a} is C₁₋₄ aliphatic. In some embodiments, R^{a} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments, R^{a} is C₁₋₄ alkyl. In some embodiments, R^{a} is C₁₋₄ alkyl optionally substituted with 1, 2, or 3 deuterium or halogen atoms. In some embodiments, R^{a} is C₁₋₄ alkyl optionally substituted with 1, 2, or 3 halogen atoms. In some embodiments, R^{a} is methyl or ethyl optionally substituted with 1, 2, or 3 halogen atoms. In some embodiments, R^{a} is methyl.

As defined generally above, R^{b} is C₁₋₄ aliphatic optionally substituted with 1, 2, or 3 deuterium or halogen atoms.

In some embodiments, R is C₁₋₄ aliphatic. In some embodiments, R^{b} is C₁₋₄ aliphatic optionally substituted with 1, 2, or 3 deuterium atoms. In some embodiments, R^{b} is C₁₋₄ aliphatic optionally substituted with 1, 2, or 3 halogen atoms.

In some embodiments, R is C₁₋₄ alkyl. In some embodiments, R^{b} is C₁₋₄ alkyl optionally substituted with 1, 2, or 3 deuterium or halogen atoms. In some embodiments, R^{b} is C₁₋₄ alkyl optionally substituted with 1, 2, or 3 halogen atoms. In some embodiments, R^{b} is methyl or ethyl optionally substituted with 1, 2, or 3 halogen atoms. In some embodiments, R^{b} is methyl.

As defined generally above, in some embodiments, R^{a} and R^{b}, taken together with the carbon atom to which they are attached, form a 3- to 8-membered cycloalkyl ring.

In some embodiments, R^{a} and R^{b}, taken together with the carbon atom to which they are attached, form a 3- to 8-membered cycloalkyl ring. In some embodiments, R^{a} and R^{b}, taken together with the carbon atom to which they are attached, form a 3- to 8-membered heterocyclyl ring containing 1-2 heteroatoms selected from nitrogen, oxygen, and sulfur.

In some embodiments, R^{a} and R^{b}, taken together with the carbon atom to which they are attached, form a cyclopropyl, cyclobutyl, or cyclopentyl ring. In some embodiments, R^{a} and R^{b}, taken together with the carbon atom to which they are attached, form an oxirane, oxetane, tetrahydrofuran, or aziridine.

In some embodiments, R^{a} and R^{b} are methyl.

In some embodiments, the pharmaceutical composition comprises a compound of formula V:

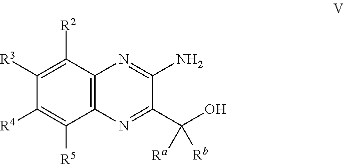

or a pharmaceutically acceptable salt thereof, wherein:
R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C)(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{a} is C₁₋₄ aliphatic optionally substituted with 1, 2, or 3 deuterium or halogen atoms;
R^{b} is C₁₋₄ aliphatic optionally substituted with 1, 2, or 3 deuterium or halogen atoms, or R^{a} and R^{b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from: C₁₋₆ aliphatic, a 3- to 8-membered saturated or partially unsaturated monocyclic carbocyclic ring, phenyl, an 8- to 10-membered bicyclic aryl ring, a 3- to 8-membered saturated or partially unsaturated monocyclic heterocyclic ring having 1-4 heteroatoms independently selected from nitrogen, oxygen, and sulfur, a 5- to 6-membered monocyclic heteroaryl ring having 1-4 heteroatoms independently selected from nitrogen, oxygen, and sulfur, a 6- to 10-membered bicyclic saturated or partially unsaturated heterocyclic ring having 1-5 heteroatoms independently selected from nitrogen, oxygen, and sulfur; or a 7- to 10-membered bicyclic heteroaryl ring having 1-5 heteroatoms independently selected from nitrogen, oxygen, and sulfur.

As described generally above, R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R² is —R. In some embodiments, R² is halogen. In some embodiments, R² is —CN. In some embodiments, R² is —OR. In some embodiments, R² is —SR. In some embodiments, R² is —N(R)₂. In some embodiments, R² is —N(R)C(O)R. In some embodiments, R² is —C(O)N(R)₂. In some embodiments, R² is —N(R)C(O)N(R)₂. In some embodiments, R² is —N(R)C(O)OR. In some embodiments, R² is —OC(O)N(R)₂. In some embodiments, R² is —N(R)S(O)₂R. In some embodiments, R² is —SO₂N(R)₂. In some embodiments, R² is —C(O)R. In some embodiments, R² is —C(O)OR. In some embodiments, R² is —OC(O)R. In some embodiments, R² is —S(O)R. In some embodiments, R² is —S(O)₂R.

In some embodiments, R² is hydrogen. In some embodiments, R² is deuterium. In some embodiments, R² is an optionally substituted C a aliphatic. In some embodiments, R² is an optionally substituted 3- to 8-membered saturated or partially unsaturated monocyclic carbocyclic ring. In some embodiments, R² is an optionally substituted phenyl. In some embodiments, R² is an optionally substituted 8- to 10-membered bicyclic aryl ring. In some embodiments, R² is an optionally substituted 3- to 8-membered saturated or partially unsaturated monocyclic heterocyclic ring having 1-4 heteroatoms independently selected from nitrogen, oxygen, and sulfur. In some embodiments, R² is an optionally substituted 5- to 6-membered monocyclic heteroaryl ring having 1-4 heteroatoms independently selected from nitrogen, oxygen, and sulfur. In some embodiments, R² is an optionally substituted 6- to 10-membered bicyclic saturated or partially unsaturated heterocyclic ring having 1-5 heteroatoms independently selected from nitrogen, oxygen, and sulfur. In some embodiments, R² is an optionally substituted 7- to 10-membered bicyclic heteroaryl ring having 1-5 heteroatoms independently selected from nitrogen, oxygen, and sulfur.

In some embodiments, R² is Cl or Br. In some embodiments, R² is Cl.

As defined generally above, R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R³ is —R. In some embodiments, R³ is halogen. In some embodiments, R is —CN. In some embodiments, R³ is —OR. In some embodiments, R³ is —SR. In some embodiments, R³ is —N(R)₂. In some embodiments, R³ is —N(R)C(O)R. In some embodiments, R³ is —C(O)N(R)₂. In some embodiments, R³ is —N(R)C(O)N(R)₂. In some embodiments, R³ is —N(R)C(O)OR. In some embodiments, R³ is —OC(O)N(R)₂. In some embodiments, R³ is —N(R)S(O)₂R. In some embodiments, R³ is —SO₂N(R)₂. In some embodiments, R³ is —C(O)R. In some embodiments, R³ is —C(O)OR. In some embodiments, R³ is —OC(O)R. In some embodiments, R³ is —S(O)R. In some embodiments, R³ is —S(O)₂R.

In some embodiments, R³ is hydrogen. In some embodiments, R³ is deuterium. In some embodiments, R³ is an optionally substituted C₁₋₆ aliphatic. In some embodiments, R³ is an optionally substituted 3- to 8-membered saturated or partially unsaturated monocyclic carbocyclic ring. In some embodiments, R³ is an optionally substituted phenyl. In some embodiments, R³ is an optionally substituted 8- to 10-membered bicyclic aryl ring. In some embodiments, KW is an optionally substituted 3- to 8-membered saturated or partially unsaturated monocyclic heterocyclic ring having 1-4 heteroatoms independently selected from nitrogen, oxygen, and sulfur. In some embodiments, R³ is an optionally substituted 5- to 6-membered monocyclic heteroaryl ring having 1-4 heteroatoms independently selected from nitrogen, oxygen, and sulfur. In some embodiments, R³ is an optionally substituted 6- to 10-membered bicyclic saturated or partially unsaturated heterocyclic ring having 1-5 heteroatoms independently selected from nitrogen, oxygen, or sulfur. In some embodiments, R³ is an optionally substituted 7- to 10-membered bicyclic heteroaryl ring having 1-5 heteroatoms independently selected from nitrogen, oxygen, and sulfur.

In some embodiments, R³ is Cl or Br. In some embodiments, R³ is Cl.

As defined generally above, R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R⁴ is —R. In some embodiments, R⁴ is halogen. In some embodiments, R⁴ is —CN. In some embodiments, R⁴ is —OR. In some embodiments, R⁴ is —SR. In some embodiments, R⁴ is —N(R)₂. In some embodiments, R⁴ is —N(R)C(O)R. In some embodiments, R⁴ is —C(O)N(R)₂. In some embodiments, R⁴ is —N(R)C(O)N(R)₂. In some embodiments, R⁴ is —N(R)C(O)OR. In some embodiments, R⁴ is —OC(O)N(R)₂. In some embodiments, R⁴ is —N(R)S(O)₂R. In some embodiments, R⁴ is —SO₂N(R)₂. In some embodiments, R⁴ is —C(O)R. In some embodiments, R⁴ is —C(O)OR. In some embodiments, R⁴ is —OC(O)R. In some embodiments, R⁴ is —S(O)R. In some embodiments, R⁴ is —S(O)₂R.

In some embodiments, R⁴ is hydrogen. In some embodiments, R⁴ is deuterium. In some embodiments, R⁴ is an optionally substituted C₆ aliphatic. In some embodiments, R⁴ is an optionally substituted 3- to 8-membered saturated or partially unsaturated monocyclic carbocyclic ring. In some embodiments, R⁴ is an optionally substituted phenyl. In some embodiments, R⁴ is an optionally substituted 8- to 10-membered bicyclic aryl ring. In some embodiments, R⁴ is an optionally substituted 3- to 8-membered saturated or partially unsaturated monocyclic heterocyclic ring having 1-4 heteroatoms independently selected from nitrogen, oxygen, and sulfur. In some embodiments, R⁴ is an optionally substituted 5- to 6-membered monocyclic heteroaryl ring having 1-4 heteroatoms independently selected from nitrogen, oxygen, and sulfur. In some embodiments, R⁴ is an optionally substituted 6- to 10-membered bicyclic saturated or partially unsaturated heterocyclic ring having 1-5 heteroatoms independently selected from nitrogen, oxygen, and sulfur. In some embodiments, R⁴ is an optionally substituted 7- to 10-membered bicyclic heteroaryl ring having 1-5 heteroatoms independently selected from nitrogen, oxygen, and sulfur.

In some embodiments, R⁴ is Cl or Br. In some embodiments, R⁴ is Cl.

As defined generally above, R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R⁵ is —R. In some embodiments, R⁵ is halogen. In some embodiments, R⁵ is —CN. In some embodiments, R⁵ is —OR. In some embodiments, R⁵ is —SR. In some embodiments, R⁵ is —N(R)₂. In some embodiments, R⁵ is —N(R)C(O)R. In some embodiments, R is —C(O)N(R)₂. In some embodiments, R⁵ is —N(R)C(O)N(R)₂. In some embodiments, R⁵ is —N(R)C(O)OR. In some embodiments, R⁵ is —OC(O)N(R)₂. In some embodiments, R⁵ is —N(R)S(O)₂R. In some embodiments, R⁵ is —SO₂N(R)₂. In some embodiments, R⁵ is —C(O)R. In some embodiments, R³ is —C(O)OR. In some embodiments, R⁵ is —OC(O)R. In some embodiments, R⁵ is —S(O)R. In some embodiments, R is —S(O)₂R.

In some embodiments, R⁵ is hydrogen. In some embodiments, R⁵ is deuterium. In some embodiments, R⁵ is an optionally substituted C₁₋₆ aliphatic. In some embodiments, R⁵ is an optionally substituted 3- to 8-membered saturated or partially unsaturated monocyclic carbocyclic ring. In some embodiments, R⁵ is an optionally substituted phenyl. In some embodiments, R⁵ is an optionally substituted 8- to 10-membered bicyclic aryl ring. In some embodiments, R⁵ is an optionally substituted 3- to 8-membered saturated or partially unsaturated monocyclic heterocyclic ring having 1-4 heteroatoms independently selected from nitrogen, oxygen, and sulfur. In some embodiments, R⁵ is an optionally substituted 5- to 6-membered monocyclic heteroaryl ring having 1-4 heteroatoms independently selected from nitrogen, oxygen, and sulfur. In some embodiments, R⁵ is an optionally substituted 6- to 10-membered bicyclic saturated or partially unsaturated heterocyclic ring having 1-5 heteroatoms independently selected from nitrogen, oxygen, and sulfur. In some embodiments, R⁵ is an optionally substituted 7- to 10-membered bicyclic heteroaryl ring having 1-5 heteroatoms independently selected from nitrogen, oxygen, and sulfur.

In some embodiments, R⁵ is Cl or Br. In some embodiments, R is Cl.

As described generally above, R^{a} is C₁₋₄ aliphatic optionally substituted with 1, 2, or 3 deuterium or halogen atoms.

In some embodiments, R^{a} is C₁₋₄ aliphatic. In some embodiments, R^{a} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments, R is C₁₋₄ alkyl. In some embodiments, R is C₁₋₄ alkyl optionally substituted with 1, 2, or 3 deuterium or halogen atoms. In some embodiments, R^{a} is C₁₋₄ alkyl optionally substituted with 1, 2, or 3 halogen atoms. In some embodiments, R^{a} is methyl or ethyl optionally substituted with 1, 2, or 3 halogen atoms. In some embodiments, R^{a} is methyl.

As defined generally above, R^{b} is C₁₋₄ aliphatic optionally substituted with 1, 2, or 3 deuterium or halogen atoms.

In some embodiments, R^{b} is C₁₋₄ aliphatic. In some embodiments, R^{b} is C₁₋₄ aliphatic optionally substituted with 1, 2, or 3 deuterium atoms. In some embodiments, R^{b} is C₁₋₄ aliphatic optionally substituted with 1, 2, or 3 halogen atoms.

In some embodiments, R is C₁₋₄ alkyl. In some embodiments, R^{b} is C₁₋₄ alkyl optionally substituted with 1, 2, or 3 deuterium or halogen atoms. In some embodiments, R^{a} is C₁₋₄ alkyl optionally substituted with 1, 2, or 3 halogen atoms. In some embodiments, R^{b} is methyl or ethyl optionally substituted with 1, 2, or 3 halogen atoms. In some embodiments, R^{b} is methyl.

As defined generally above, in some embodiments, R^{a} and R^{b}, taken together with the carbon atom to which they are attached, form a 3- to 8-membered cycloalkyl or heterocyclyl ring containing 1-2 heteroatoms selected from nitrogen, oxygen, and sulfur.

In some embodiments, R^{a} and R^{b}, taken together with the carbon atom to which they are attached, form a 3- to 8-membered cycloalkyl ring. In some embodiments, R^{a} and R^{b}, taken together with the carbon atom to which they are attached, form a 3- to 8-membered heterocyclyl ring containing 1-2 heteroatoms selected from nitrogen, oxygen, and sulfur.

In some embodiments, R^{a} and R^{b}, taken together with the carbon atom to which they are attached, form a cyclopropyl, cyclobutyl, or cyclopentyl ring. In some embodiments, R^{a} and R^{b}, taken together with the carbon atom to which they are attached, form an oxirane, oxetane, tetrahydrofuran, or aziridine.

In some embodiments, R^{a} and R^{b} are methyl.

In some embodiments, the pharmaceutical composition comprises a compound of formula VI-a, VI-b, VI-c, or VI-d:

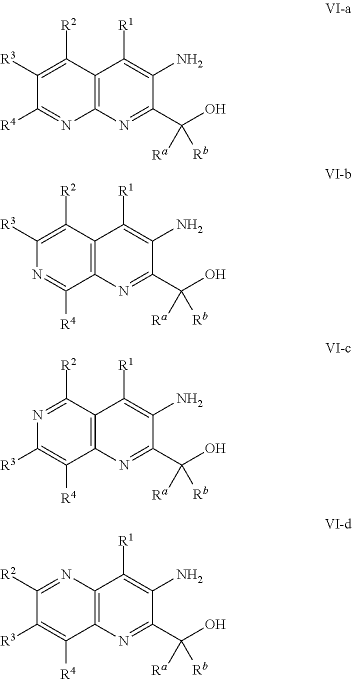

or a pharmaceutically acceptable salt thereof, wherein

each of R¹, R², R³, R⁴, R^{a}, and R^{b} is as defined and described in embodiments herein, both singly and in combination.

In some embodiments, the compound is of formula VI-a above.

In some embodiments, R¹ and R⁴ are H.

In some embodiments, R² is H.

In some embodiments, R^{a} and R^{b} are C₁₋₄ alkyl optionally substituted with 1, 2, or 3 deuterium or halogen atoms, or R^{a} and R^{b} are taken together with the carbon to which they are attached to form a 3-8 membered cycloalkyl or heterocyclyl ring containing 1-2 heteroatoms selected from nitrogen, oxygen, and sulfur.

In some embodiments, R³ is H, C₁₋₄ alkyl, halogen, —NR, —OR, —SR, —CO₂R, or —C(O)R, wherein R is H, optionally substituted C₁₋₄ alkyl, or optionally substituted phenyl.

In another aspect, the compound is a compound of formula VI-e, VI-f, VI-g, or VI-h:

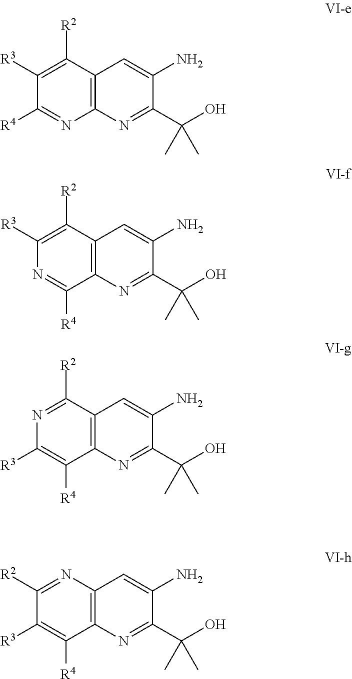

or a pharmaceutically acceptable salt thereof, wherein:
each of R², R³, and R⁴ is as defined above and described in embodiments herein, both singly and in combination.

In another aspect, the compound is a compound of formula VI-i, VI-j, VI-k, VI-l, VI-m, or VI-n:

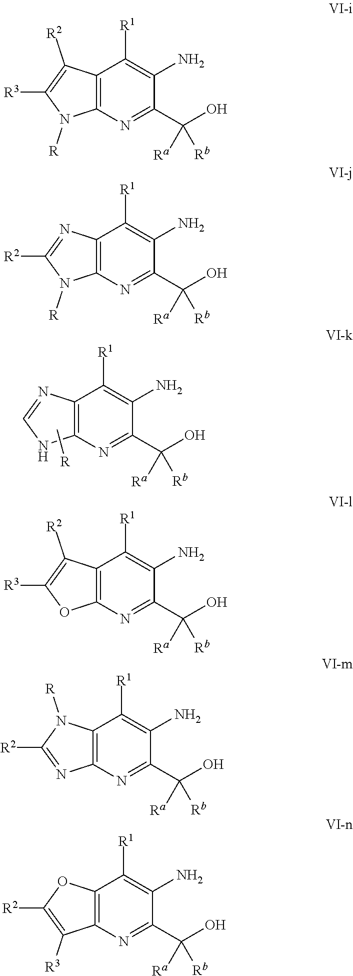

or a pharmaceutically acceptable salt thereof, wherein:
each of R, R¹, R², R³, R^{a}, and R^{b} is as defined above and described in embodiments herein, both singly and in combination.

In another aspect, the pharmaceutical composition comprises a compound of formula VII-a:

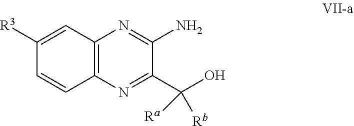

or a pharmaceutically acceptable salt thereof, wherein:
each of R³, R^{a}, and R^{b} is as defined above and described in embodiments herein, both singly and in combination.

In some embodiments, the compound is a compound of formula I selected from those depicted in Table 1a, below:

TABLE 1a

| | |
|---|---|
| 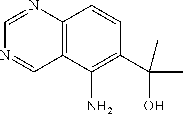 | I-1 |
| 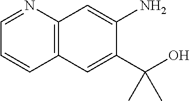 | I-2 |
| 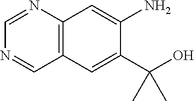 | I-3 |
| 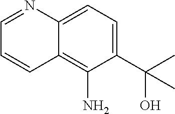 | I-4 |
| 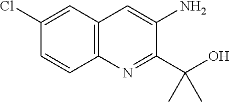 [SMILES: CC(C)(O)c1nc2ccc(Cl)cc2cc1N] | I-5 |
| 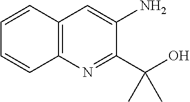 | I-6 |
| 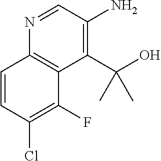 [SMILES: CC(C)(O)c1c(N)cnc2ccc(Cl)c(F)c12] | I-7 |
| 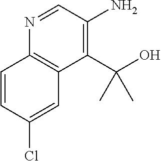 [SMILES: CC(C)(O)c1c(N)cnc2ccc(Cl)cc12] | I-8 |
| 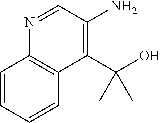 | I-9 |
| 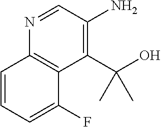 [SMILES: CC(C)(O)c1c(N)cnc2cccc(F)c12] | I-10 |
| 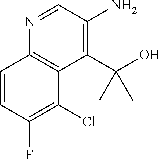 [SMILES: CC(C)(O)c1c(N)cnc2ccc(F)c(Cl)c12] | I-11 |
| 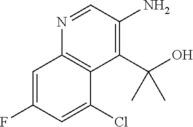 [SMILES: CC(C)(O)c1c(N)cnc2cc(F)cc(Cl)c12] | I-12 |
| 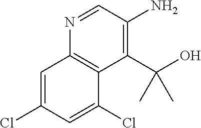 [SMILES: CC(C)(O)c1c(N)cnc2cc(Cl)cc(Cl)c12] | I-13 |
| 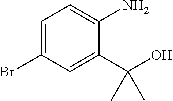 | I-14 |
| 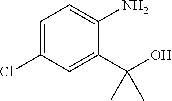 | I-15 |
| 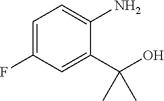 | I-16 |
| 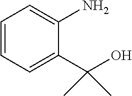 | I-17 |
| 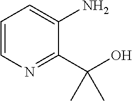 | I-18 |
| 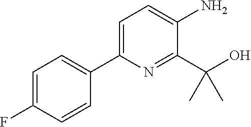 [SMILES: CC(C)(O)c1nc(-c2ccc(F)cc2)ccc1N] | I-19 |
| 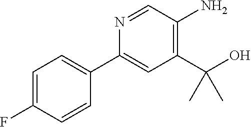 [SMILES: CC(C)(O)c1cc(-c2ccc(F)cc2)ncc1N] | I-20 |
| 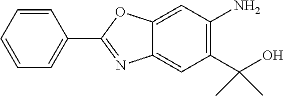 [SMILES: CC(C)(O)c1cc2nc(-c3ccccc3)oc2cc1N] | I-21 |
| 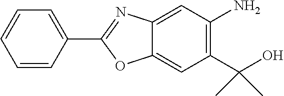 [SMILES: CC(C)(O)c1cc2oc(-c3ccccc3)nc2cc1N] | I-22 |
| 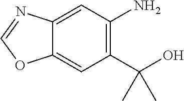 | I-23 |
| 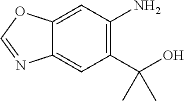 | I-24 |
| 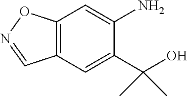 | I-25 |
| 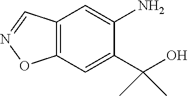 | I-26 |
| 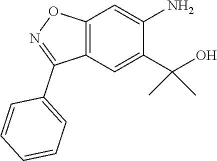 [SMILES: CC(C)(O)c1cc2c(-c3ccccc3)noc2cc1N] | I-27 |
| 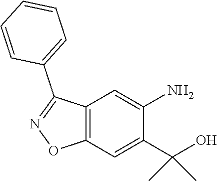 [SMILES: CC(C)(O)c1cc2onc(-c3ccccc3)c2cc1N] | I-28 |
| 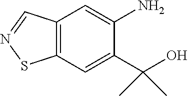 | I-29 |
| 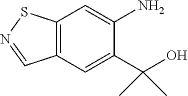 | I-30 |
| 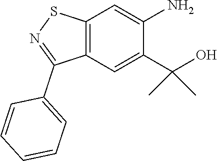 [SMILES: CC(C)(O)c1cc2c(-c3ccccc3)nsc2cc1N] | I-31 |
| 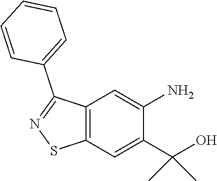 [SMILES: CC(C)(O)c1cc2snc(-c3ccccc3)c2cc1N] | I-32 |
| 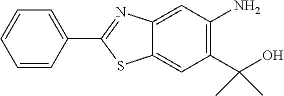 [SMILES: CC(C)(O)c1cc2sc(-c3ccccc3)nc2cc1N] | I-33 |
| 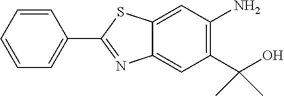 [SMILES: CC(C)(O)c1cc2nc(-c3ccccc3)sc2cc1N] | I-34 |
| 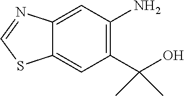 | I-35 |
| 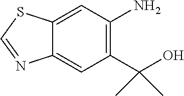 | I-36 |
| 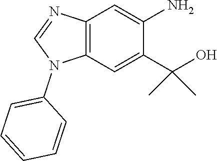 [SMILES: CC(C)(O)c1cc2c(cc1N)ncn2-c1ccccc1] | I-37 |
| 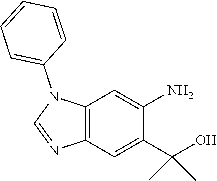 [SMILES: CC(C)(O)c1cc2ncn(-c3ccccc3)c2cc1N] | I-38 |
| 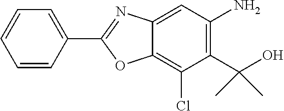 [SMILES: CC(C)(O)c1c(N)cc2nc(-c3ccccc3)oc2c1Cl] | I-39 |
| 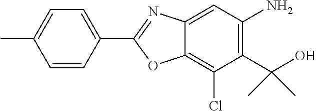 [SMILES: Cc1ccc(-c2nc3cc(N)c(C(C)(C)O)c(Cl)c3o2)cc1] | I-40 |
| 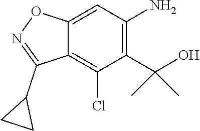 [SMILES: CC(C)(O)c1c(N)cc2onc(C3CC3)c2c1Cl] | I-41 |
| 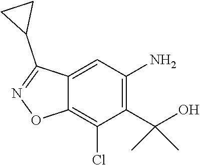 [SMILES: CC(C)(O)c1c(N)cc2c(C3CC3)noc2c1Cl] | I-42 |
| 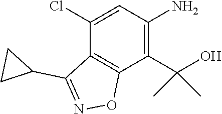 [SMILES: CC(C)(O)c1c(N)cc(Cl)c2c(C3CC3)noc12] | I-43 |
| 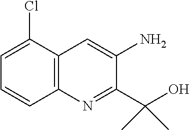 [SMILES: CC(C)(O)c1nc2cccc(Cl)c2cc1N] | I-44 |
| 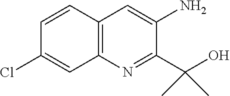 [SMILES: CC(C)(O)c1nc2cc(Cl)ccc2cc1N] | I-45 |
| 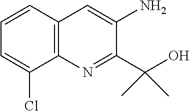 [SMILES: CC(C)(O)c1nc2c(Cl)cccc2cc1N] | I-46 |
| 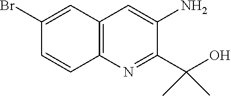 [SMILES: CC(C)(O)c1nc2ccc(Br)cc2cc1N] | I-47 |
| 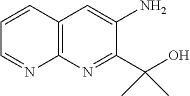 | I-48 |
| 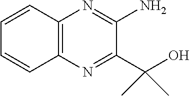 | I-49 |
| 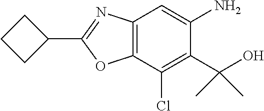 [SMILES: CC(C)(O)c1c(N)cc2nc(C3CCC3)oc2c1Cl] | I-50 |
| 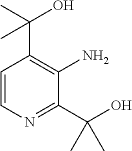 | II-51 |
| 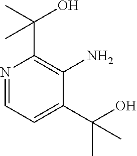 | I-52 |

or a pharmaceutically acceptable salt thereof.

In some embodiments, the compound is selected from

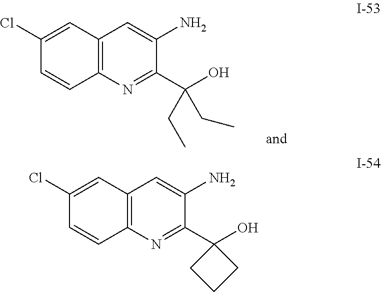

or a pharmaceutically acceptable salt thereof.

In some embodiments, the compound is selected from the compounds depicted in Table 1b, below.

TABLE 1b

| | |
|---|---|
| 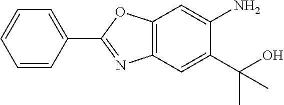 [SMILES: CC(C)(O)c1cc2nc(-c3ccccc3)oc2cc1N] | I-21 |
| 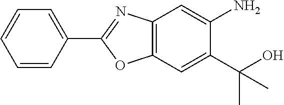 [SMILES: CC(C)(O)c1cc2oc(-c3ccccc3)nc2cc1N] | I-22 |
| 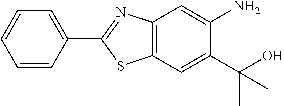 [SMILES: CC(C)(O)c1cc2sc(-c3ccccc3)nc2cc1N] | I-33 |
| 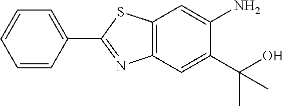 [SMILES: CC(C)(O)c1cc2nc(-c3ccccc3)sc2cc1N] | I-34 |
| 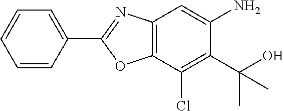 [SMILES: CC(C)(O)c1c(N)cc2nc(-c3ccccc3)oc2c1Cl] | I-39 |
| 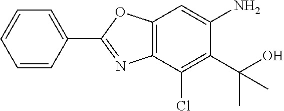 [SMILES: CC(C)(O)c1c(N)cc2oc(-c3ccccc3)nc2c1Cl] | I-39b |
| 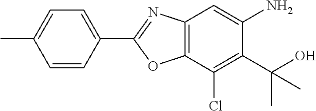 [SMILES: Cc1ccc(-c2nc3cc(N)c(C(C)(C)O)c(Cl)c3o2)cc1] | I-40 |
| 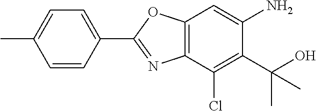 [SMILES: Cc1ccc(-c2nc3c(Cl)c(C(C)(C)O)c(N)cc3o2)cc1] | I-40b |
| 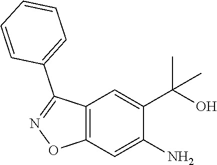 [SMILES: CC(C)(O)c1cc2c(-c3ccccc3)noc2cc1N] | I-27 |
| 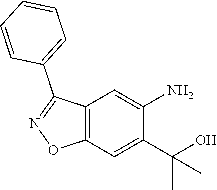 [SMILES: CC(C)(O)c1cc2onc(-c3ccccc3)c2cc1N] | I-28 |
| 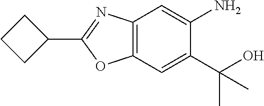 [SMILES: CC(C)(O)c1cc2oc(C3CCC3)nc2cc1N] | I-55 |
| 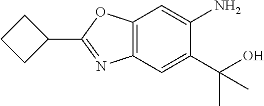 [SMILES: CC(C)(O)c1cc2nc(C3CCC3)oc2cc1N] | I-56 |
| 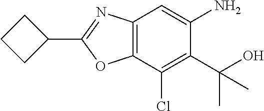 [SMILES: CC(C)(O)c1c(N)cc2nc(C3CCC3)oc2c1Cl] | I-50 |
| 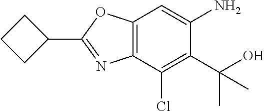 [SMILES: CC(C)(O)c1c(N)cc2oc(C3CCC3)nc2c1Cl] | I-50b |
| 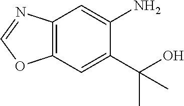 | I-23 |
| 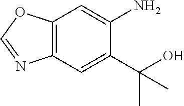 | I-24 |
| 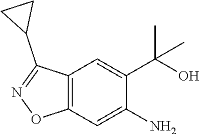 [SMILES: CC(C)(O)c1cc2c(C3CC3)noc2cc1N] | I-58 |
| 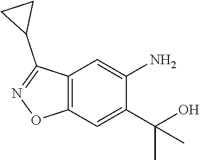 [SMILES: CC(C)(O)c1cc2onc(C3CC3)c2cc1N] | I-59 |
| 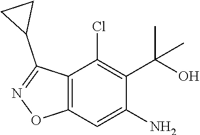 [SMILES: CC(C)(O)c1c(N)cc2onc(C3CC3)c2c1Cl] | I-41 |
| 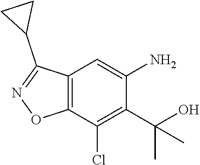 [SMILES: CC(C)(O)c1c(N)cc2c(C3CC3)noc2c1Cl] | I-42 |
| 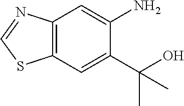 | I-60 |
| 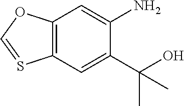 | I-60 |

or a pharmaceutically acceptable salt thereof.

In some embodiments, the compound is selected from the compounds depicted in Table 1c, below.

TABLE 1c
Exemplary Compounds of Formula IV

| | |
|---|---|
| 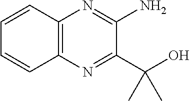 | I-49 |
| 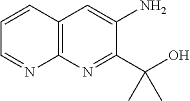 | I-48 |
| 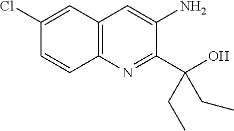 [SMILES: CCC(O)(CC)c1nc2ccc(Cl)cc2cc1N] | I-53 |
| 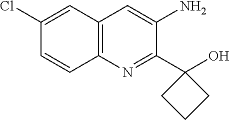 [SMILES: Nc1cc2cc(Cl)ccc2nc1C1(O)CCC1] | I-54 |
| 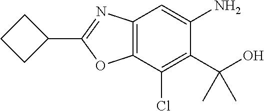 [SMILES: CC(C)(O)c1c(N)cc2nc(C3CCC3)oc2c1Cl] | I-50 |
| 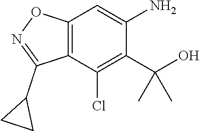 [SMILES: CC(C)(O)c1c(N)cc2onc(C3CC3)c2c1Cl] | I-41 |
| 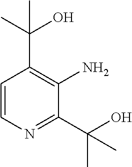 | I-52 |
| 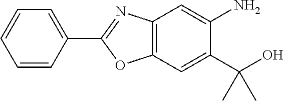 [SMILES: CC(C)(O)c1cc2oc(-c3ccccc3)nc2cc1N] | I-22 |
| 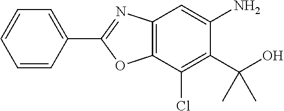 [SMILES: CC(C)(O)c1c(N)cc2nc(-c3ccccc3)oc2c1Cl] | I-39 |
| 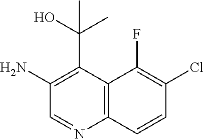 [SMILES: CC(C)(O)c1c(N)cnc2ccc(Cl)c(F)c12] | I-7 |
| 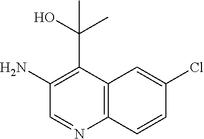 [SMILES: CC(C)(O)c1c(N)cnc2ccc(Cl)cc12] | I-8 |

or a pharmaceutically acceptable salt thereof.

In some embodiments, the pharmaceutical composition comprises a compound of formula VIII:

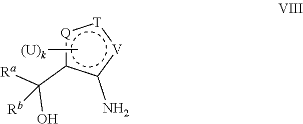

or a pharmaceutically acceptable salt thereof, wherein:
each Q, T, and V is independently selected from N or NH, S, O, CU, and CH;

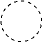

represents two double bonds within the ring, which comply with the valency requirements of the atoms and heteroatoms present in the ring;
k is 0, 1, 2, or 3;
R^{a} is C₁₋₄ aliphatic optionally substituted with 1, 2, or 3 deuterium or halogen atoms;
R^{b} is C₁₋₄ aliphatic optionally substituted with 1, 2, or 3 deuterium or halogen atoms; or
R^{a} and R^{b}, taken together with the carbon atom to which they are attached, form a 3- to 8-membered cycloalkyl or heterocyclyl ring containing 1-2 heteroatoms selected from nitrogen, oxygen, and sulfur;
each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
two occurrences of U on adjacent carbon atoms can form an optionally substituted fused ring, selected from a fused phenyl ring: a fused 5- to 6-membered saturated or partially unsaturated heterocyclic ring containing 1-3 heteroatoms independently selected from nitrogen, oxygen, and sulfur; and a fused 5- to 6-membered heteroaryl ring containing 1-3 heteroatoms independently selected from nitrogen, oxygen, and sulfur; and
each R is independently selected from hydrogen, deuterium, or an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; or a 7- to 10-membered bicyclic heteroaryl ring having 1-5 heteroatoms independently selected from nitrogen, oxygen, and sulfur.

Each of k, U, and R is as defined and described above.

As defined above and described herein, Q is selected from N or NH, S, O, CU, and CH. In some embodiments, Q is N or NH. In some embodiments, Q is S. In some embodiments, Q is O. In some embodiments, Q is CU. In some embodiments, Q is CH.

As defined above and described herein, T is selected from N or NH, S, O, CU, and CH. In some embodiments, T is N or NH. In some embodiments, T is S. In some embodiments, T is O. In some embodiments, T is CU. In some embodiments, T is CH.

As defined above and described herein. V is selected from N or NH, S, O, CU, and CH. In some embodiments, V is N or NH. In some embodiments, V is S. In some embodiments, V is O. In some embodiments, V is CU. In some embodiments, V is CH.

As defined above and described herein, k is 0, 1, 2, or 3. In some embodiments, k is 0. In some embodiments, k is 1. In some embodiments, k is 2. In some embodiments, k is 3.

As defined above and described herein,

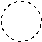

represents two double bonds within the ring, which comply with the valency requirements of the atoms and heteroatoms present in the ring. In some embodiments, the ring formed is thiophene. In some embodiments, the ring formed is oxazole.

In some embodiments, the ring formed is isothiazole.

In some embodiments, one or more of Q and V are CH; T is S;

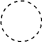

is arranged to form a thiophene; and k is 0. In some embodiments, one or more of Q is CH; T is N or NH; V is O;

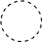

is arranged to form an isoxazole; and k is 0. In some embodiments, one or more of Q is S; T and V are CH;

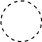

is arranged to form a thiophene; k is 1; and U is —S(O)₂R. In some embodiments, one or more of Q is S; T and V are CH;

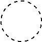

is arranged to form a thiophene; k is 1; and U is —S(O)₂CH₃. In some embodiments, one or more of Q is CH; T is N or NH; V is S;

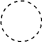

is arranged to form an isothiazole; and k is 0.

In some embodiments, the compound of formula VIII is selected from those depicted in Table 2, below:

TABLE 2
Exemplary Compounds of Formula VIII

| | |
|---|---|
| 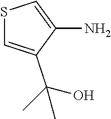 | VIII-1 |
| 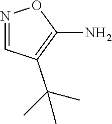 | VIII-2 |
| 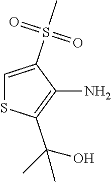 | VIII-3 |
| 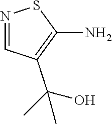 | VIII-4 |

In some embodiments, the compound is a compound of formula IX-A or IX-B:

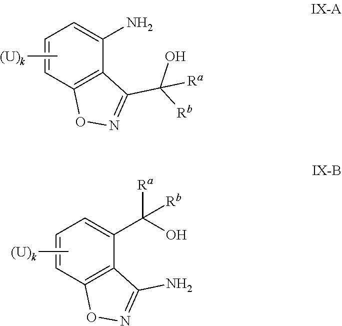

or a pharmaceutically acceptable salt thereof, wherein:
k is 0, 1, 2, or 3;
R^{a} is C₁₋₄ aliphatic optionally substituted with 1, 2, or 3 deuterium or halogen atoms;
R^{b} is C₁₋₄ aliphatic optionally substituted with 1, 2, or 3 deuterium or halogen atoms; or
R^{a} and R^{b}, taken together with the carbon atom to which they are attached, form a 3- to 8-membered cycloalkyl or heterocyclyl ring containing 1-2 heteroatoms selected from nitrogen, oxygen, and sulfur;
each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
two occurrences of U on adjacent carbon atoms can form an optionally substituted fused ring, selected from a fused phenyl ring; a fused 5- to 6-membered saturated or partially unsaturated heterocyclic ring containing 1-3 heteroatoms independently selected from nitrogen, oxygen, and sulfur; or a fused 5- to 6-membered heteroaryl ring containing 1-3 heteroatoms independently selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

Each of k, U, and R is as defined and described above.

In some embodiments, the compound of formula IX-A or IX-B is selected from those depicted in Table 3, below:

TABLE 3
Exemplary Compounds of Formula IX

| | |
|---|---|
| 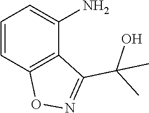 | IX-1 |
| 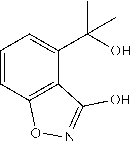 | IX-2 |

### 4.3. Deuterated Compounds

In some embodiments, the compound is a deuterated form of a compound above or a pharmaceutically acceptable salt thereof.

In some embodiments, the pharmaceutical composition comprises a compound of formula X:

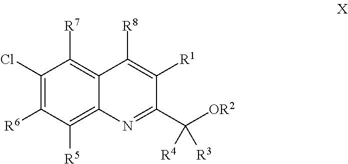

or a pharmaceutically acceptable salt thereof, wherein:

R¹ is selected from —NH₂, —NHD, and —ND₂;

R² is selected from hydrogen or deuterium;

R³ and R⁴ are independently selected from —CH₃, —CH₂D, —CHD₂, and —CD₃; and

R⁵, R⁶, R⁷, and R⁸ are each independently selected from hydrogen and deuterium;

provided that at least one of R¹, R², R³, R⁴, R⁵, R⁶, R⁷, or R⁸ is or contains deuterium.

In some embodiments, the compound is a compound of formula X-A:

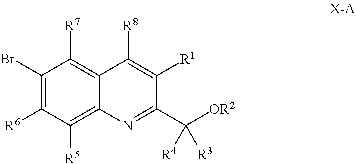

or a pharmaceutically acceptable salt thereof, wherein:

R¹ is selected from —NH₂, —NHD, and —ND₂;

R² is selected from hydrogen and deuterium;

R³ and R⁴ are independently selected from —CH₃, —CH₂D, —CHD₂, and —CD₃; and

R⁵, R⁶, R⁷, and R⁸ are each independently selected from hydrogen and deuterium;

provided that at least one of R¹, R², R³, R⁴, R⁵, R⁶, R⁷, or R⁸ is or contains deuterium.

In some embodiments, the compound is a compound of formula XI-A or XI-B:

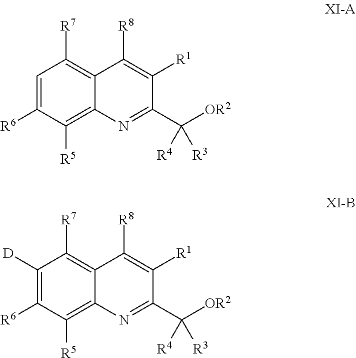

or a pharmaceutically acceptable salt thereof, wherein:

R¹ is selected from —NH₂, —NHD, and —ND₂;

R² is selected from hydrogen and deuterium;

R³ and R⁴ are independently selected from —CH₃, —CH₂D, —CHD₂, and —CD₃; and

R⁵, R⁶, R⁷ and R⁸ are each independently selected from hydrogen and deuterium;

provided that at least one of R¹, R², R³, R⁴, R⁵, R⁶, R⁷, or R⁸ in formula XI-A is or contains deuterium.

In some embodiments, the compound is a compound of formula XII-A, XII-B, or XII-C:

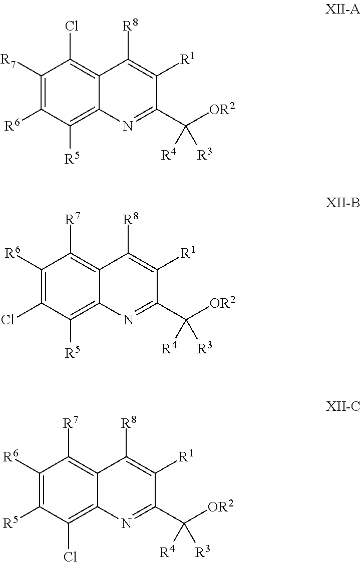

or a pharmaceutically acceptable salt thereof, wherein:

R¹ is selected from —NH₂, —NHD, and —ND₂;

R² is selected from hydrogen and deuterium;

R³ and R⁴ are independently selected from —CH₃, —CH₂D, —CHD₂, and —CD₃; and

R⁵, R⁶, R⁷, and R⁸ are each independently selected from hydrogen and deuterium;

provided that at least one of R¹, R², R³, R⁴, R⁵, R⁶, R⁷, or R⁸ is or contains deuterium.

In some embodiments, the compound is a compound of formula XII:

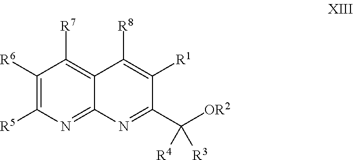

or a pharmaceutically acceptable salt thereof, wherein:

R¹ is selected from —NH₂, —NHD, and —ND₂;

R² is selected from hydrogen and deuterium;

R³ and R⁴ are independently selected from —CH₃, —CH₂D, —CHD₂, and —CD₃; and

R⁵, R⁶, R⁷, and R⁸ are each independently selected from hydrogen and deuterium;

provided that at least one of R¹, R², R³, R⁴, R⁵, R⁶, R⁷, or R⁸ is or contains deuterium.

In some embodiments, the compound is a compound of formula XIV:

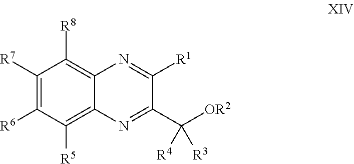

or a pharmaceutically acceptable salt thereof, wherein:

R¹ is selected from —NH₂, —NHD, and —ND₂;

R² is selected from hydrogen and deuterium;

R³ and R⁴ are independently selected from —CH₃, —CH₂D, —CHD₂, and —CD₃; and

R⁵, R⁶, R⁷, and R⁸ are each independently selected from hydrogen and deuterium;

provided that at least one of R¹, R², R³, R⁴, R⁵, R⁶, R⁷, or R⁸ is or contains deuterium.

In some embodiments, the compound is a compound of formula XV-A or XV-B:

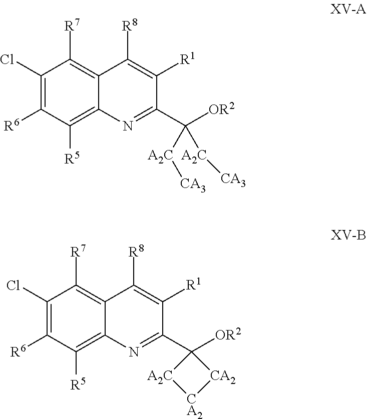

or a pharmaceutically acceptable salt thereof, wherein:

each A is independently hydrogen and deuterium;

R¹ is selected from —NH₂, —NHD, and —ND₂;

R² is selected from hydrogen and deuterium; and

R⁵, R⁶, R⁷, and R⁸ are each independently selected from hydrogen and deuterium;

provided that at least one of A, R¹, R², R⁵, R⁶, R⁷, or R⁸ is or contains deuterium.

In some embodiments, the compound is a compound of formula XVI-A or XVI-B:

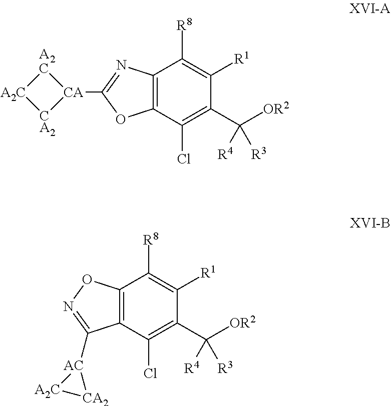

or a pharmaceutically acceptable salt thereof, wherein:

each A is independently hydrogen or deuterium;

R¹ is selected from —N₂, —NHD, and —ND₂;

R² is selected from hydrogen and deuterium;

R³ and R⁴ are independently selected from —CH₃, —CH₂D, —CHD₂, and —CD₃; and

R⁸ is selected from hydrogen and deuterium.

In some embodiments, the compound is a compound of formula XVII-A or XVII-B:

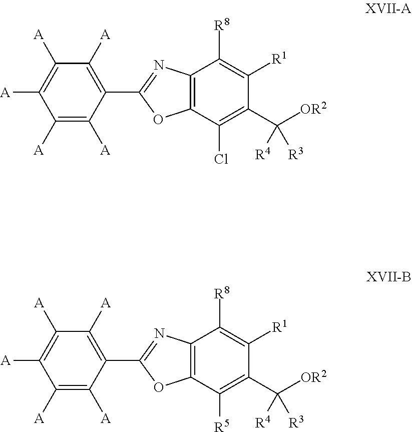

or a pharmaceutically acceptable salt thereof, wherein:

each A is independently hydrogen and deuterium;

R¹ is selected from —NH₂, —NHD, and —ND₂;

R² is selected from hydrogen and deuterium;

R³ and R⁴ are independently selected from —CH₃, —CH₂D, —CHD₂, and —CD₃; and

R⁵ and R⁸ are each independently selected from hydrogen and deuterium;

provided that at least one of A, R¹, R², R³, R⁴, R⁵, or R⁸ is or contains deuterium.

In some embodiments, the compound is a compound of formula XVIII-A or XVIII-B:

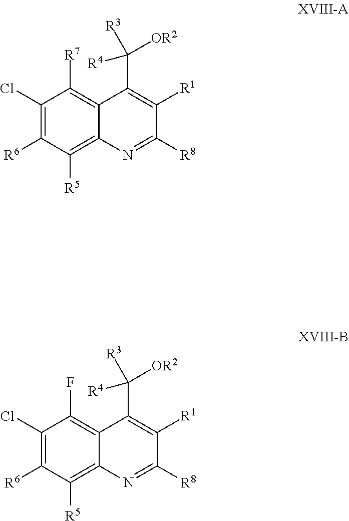

or a pharmaceutically acceptable salt thereof, wherein:
R¹ is selected from —NH, —NHD, and —ND₂;

R² is selected from hydrogen and deuterium;

R³ and R⁴ are independently selected from —CH₃, —CH₂D, —CHD₂, and —CD₃; and

R⁵, R⁶, R⁷, and R⁸ are each independently selected from hydrogen and deuterium;

provided that at least one of R¹, R², R³, R⁴, R⁵, R⁶, R⁷ or R⁸ is or contains deuterium.

In some embodiments, the compound is a compound of formula XIX:

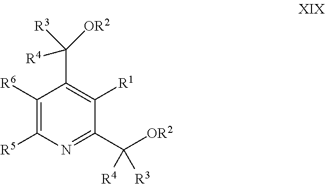

or a pharmaceutically acceptable salt thereof, wherein:

R¹ is selected from —NH₂, —NHD, and —ND₂;

R² is selected from hydrogen and deuterium;

R³ and R⁴ are independently selected from —CH₃, —CH₂D, —CHD₂, and —CD₃; and

R⁵ and R⁶ are each independently selected from hydrogen and deuterium;

provided that at least one of R¹, R², R³, R⁴, R⁵, or R⁶ is or contains deuterium.

The following embodiments are applicable to each of the preceding formula X-XIX.

As defined above and described herein, R¹ is selected from —NH₂, —NHD, and —ND₂.

In some embodiments, R¹ is —NH₂. In some embodiments, R¹ is —NH₂ and at least one of R², R³, R⁴, R⁵, R⁶, R⁷, or R⁸ is or contains deuterium.

In some embodiments, R¹ is —NHD. In some embodiments, R¹ is —NHD and at least one of R², R³, R⁴, R⁵, R⁶, R⁷, or R⁸ is or contains deuterium.

In some embodiments, R¹ is —ND₂. In some embodiments, R¹ is —ND₂ and at least one of R², R³, R⁴, R⁵, R⁶, R⁷, or R⁸ is or contains deuterium.

As defined above and described herein, A is selected from hydrogen and deuterium.

In some embodiments, A is hydrogen. In some embodiments, A is hydrogen and at least one of R¹, R³, R⁴, R⁵, R⁶, R⁷ or R⁸ is or contains deuterium. In some embodiments, A is deuterium. In some embodiments, A is deuterium and at least one of R¹, R³, R⁴, R⁵, R⁶, R⁷, or R⁸ is or contains deuterium.

As defined above and described herein, R² is selected from hydrogen and deuterium.

In some embodiments, R² is hydrogen. In some embodiments, R² is hydrogen and at least one of R¹, R³, R⁴, R⁵, R⁶, R⁷, or R⁸ is or contains deuterium. In some embodiments, R² is deuterium. In some embodiments, R² is deuterium and at least one of R¹, R³, R⁴, R⁵, R⁶, R⁷, or R⁸ is or contains deuterium.

As defined above and described herein, R³ is selected from —CH₃, —CH₂D, —CHD₂, and —CD₃.

In some embodiments, R³ is —CH₃. In some embodiments, R³ is —CH₃ and at least one of R¹, R², R⁴, R⁵, R⁶, R⁷, or R⁸ is or contains deuterium.

In some embodiments, R³ is —CH₂D. In some embodiments, R³ is —CH₂D and at least one of R¹, R², R⁴, R⁵, R⁶, R⁷, or R⁸ is or contains deuterium.

In some embodiments, R³ is —CHD₂. In some embodiments, R³ is —CH₂D₂ and at least one of R¹, R², R⁴, R⁵, R⁶, R⁷, or R⁸ is or contains deuterium.

In some embodiments, R³ is —CD₃. In some embodiments, R³ is —CD₃ and at least one of R¹, R², R⁴, R⁵, R⁶, R⁷, or R⁸ is or contains deuterium.

As defined above and described herein, R⁴ is selected from —CH₃, CH₂D, —CHD₂, and —CD₃.

In some embodiments, R⁴ is —CH₃. In some embodiments, R⁴ is —CH₃ and at least one of R¹, R², R³, R⁵, R⁶, R⁷, or R⁸ is or contains deuterium.

In some embodiments, R⁴ is —CH₂D. In some embodiments, R⁴ is —CH₂D and at least one of R¹, R², R³, R⁵, R⁶, R⁷, or R⁸ is or contains deuterium.

In some embodiments, R⁴ is —CHD₂. In some embodiments, R⁴ is —CHD₂ and at least one of R¹, R², R³, R⁵, R⁶, R⁷, or R⁸ is or contains deuterium.

In some embodiments, R⁴ is —CD₃. In some embodiments, R⁴ is —CD₃ and at least one of R¹, R², R³, R⁵, R⁶, R⁷, or R⁸ is or contains deuterium.

As defined above and described herein, R⁵ is selected from hydrogen and deuterium.

In some embodiments, R⁵ is hydrogen. In some embodiments, R⁵ is hydrogen and at least one of R¹, R², R³, R⁴, R⁶, R⁷, or R⁸ is or contains deuterium. In some embodiments, R⁵ is deuterium. In some embodiments, R⁵ is deuterium and at least one of R¹, R², R³, R⁴, R⁶, R⁷, or R⁸ is or contains deuterium.

As defined above and described herein, R⁶ is selected from hydrogen and deuterium.

In some embodiments, R⁶ is hydrogen. In some embodiments, R⁶ is hydrogen and at least one of R¹, R², R³, R⁴, R⁵, R⁷, or R⁸ is or contains deuterium. In some embodiments, R⁶ is deuterium. In some embodiments, R⁶ is deuterium and at least one of R¹, R², R³, R⁴, R⁵, R⁷, or R⁸ is or contains deuterium.

As defined above and described herein, R⁷ is selected from hydrogen and deuterium.

In some embodiments, R⁷ is hydrogen. In some embodiments, R⁷ is hydrogen and at least one of R¹, R², R³, R⁴, R⁵, or R⁸ is or contains deuterium. In some embodiments, R⁷ is deuterium. In some embodiments, R⁸ is deuterium and at least one of R¹, R², R³, R⁴, R⁵, R⁶, or R⁸ is or contains deuterium.

As defined above and described herein, R⁸ is selected from hydrogen and deuterium.

In some embodiments, R⁸ is hydrogen. In some embodiments, R⁸ is hydrogen and at least one of R¹, R², R³, R⁴, R⁵, R⁶, or R⁷ is or contains deuterium. In some embodiments, R⁸ is deuterium. In some embodiments, R⁸ is deuterium and at least one of R¹, R², R³, R⁴, R⁵, R⁶, or R⁷ is or contains deuterium.

In some embodiments, the compound is a compound of formula X, X-A, XI-A, XI-B, XII-A, XII-B, XII-C, XIII, or XIV, or a pharmaceutically acceptable salt thereof, wherein each of R³, R⁴, R⁵, R⁶, R⁷, and R⁸ is as defined above and described herein, and wherein each of R¹ and R² is as defined in an entry set forth in Table 4a, below.

TABLE 4a
Exemplary Compounds of Formula X, X-A, XI-A, XI-B,
XII-A, XII-B, XII-C, XIII, or XIV

| Entry | R¹ | R² |
|---|---|---|
| i | —NH₂ | H |
| ii | —NH₂ | D |
| iii | —NHD | H |
| iv | —NHD | D |
| v | —ND₂ | H |
| vi | —ND₂ | D |

In some embodiments, the compound is a compound of formula X, X-A, XI-A, XI-B, XII-A, XII-B, XII-C, XIII, or XIV, or a pharmaceutically acceptable salt thereof, wherein each of R¹, R², R⁵, R⁶, R⁷, and R⁸ is as defined above and described herein, and wherein each of R³ and R⁴ is as defined in an entry set forth in Table 4b, below.

TABLE 4b
Exemplary Compounds of Formula X, X-A,
XI-A, XI-B, XII-A, XII-B, XII-C, XIII, or XIV

| Entry | R³ | R⁴ |
|---|---|---|
| i | —CH₃ | —CH₃ |
| ii | —CH₃ | —CH₂D |
| iii | —CH₃ | —CHD₂ |
| iv | —CH₃ | —CD₃ |
| v | —CH₂D | —CH₃ |
| vi | —CH₂D | —CH₂D |
| vii | —CH₂D | —CHD₂ |
| viii | —CH₂D | —CD₃ |
| ix | —CHD₂ | —CH₃ |
| x | —CHD₂ | —CH₂D |
| xi | —CHD₂ | —CHD₂ |
| xii | —CHD₂ | —CD₃ |
| xiii | —CD₃ | —CH₃ |
| xiv | —CD₃ | —CH₂D |
| xv | —CD₃ | —CHD₂ |
| xi | —CD₃ | —CD₃ |

In some embodiments, the compound is a compound of formula X, X-A, XI-A, XI-B, XII-A, XII-B, XII-C, XIII, or XIV, or a pharmaceutically acceptable salt thereof, wherein each of R¹, R², R³, and R⁴ is as defined above and described herein, and wherein each of R⁵, R⁶, R⁷, and R⁸ is as defined in an entry set forth in Table 4c, below.

TABLE 4c
ExemplaryCompounds of Formula X, X-A, XI-A,
XI-B, XII-A, XII-B, XII-C, XIII, or XIV

| Entry | R⁵ | R⁶ | R⁷ | R⁸ |
|---|---|---|---|---|
| i | H | H | H | H |
| ii | H | H | H | D |
| iii | H | H | D | H |
| iv | H | D | H | H |
| v | D | H | H | H |
| vi | H | H | D | D |
| vii | H | D | H | D |
| viii | D | H | H | D |
| ix | H | D | D | H |
| x | D | H | D | H |
| xi | D | D | H | H |
| xii | H | D | D | D |
| xiii | D | H | D | D |
| xiv | D | D | H | D |
| xv | D | D | D | H |
| xvi | D | D | D | D |

In some embodiments, the compound is a compound of formula X, X-A, XI-A, XI-B, XII-A, XII-B, XII-C, XIII, or XIV, or a pharmaceutically acceptable salt thereof, wherein each of R¹ and R² is as defined in an entry set forth in Table 4a, above, each of R³ and R⁴ is as defined in an entry set forth in Table 4b, above, and each of R⁵, R⁶, R⁷, and R⁸, is as defined in an entry set forth in Table 4c, above.

In some embodiments, the pharmaceutical composition comprises a compound selected from those recited in any of Table 4a, Table 4b, or Table 4c, or a pharmaceutically acceptable salt thereof.

In some embodiments, the pharmaceutical composition comprises a compound of formula X selected from the compounds depicted in Table 5, below.

TABLE 5
Representative Compounds of Formula X

| | |
|---|---|
| 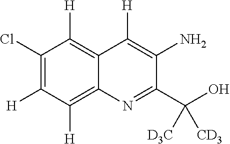 [SMILES: [2H]C([2H])([2H])C(O)(c1nc2ccc(Cl)cc2cc1N)C([2H])([2H])[2H]] | X-1 |
| 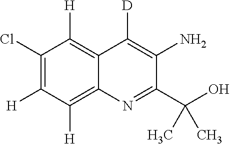 [SMILES: [2H]c1c(N)c(C(C)(C)O)nc2ccc(Cl)cc12] | X-2 |
| 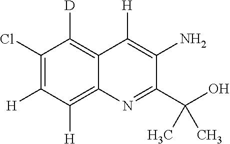 [SMILES: [2H]c1c(Cl)ccc2nc(C(C)(C)O)c(N)cc12] | X-3 |
| 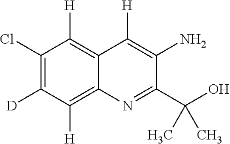 [SMILES: [2H]c1cc2nc(C(C)(C)O)c(N)cc2cc1Cl] | X-4 |
| 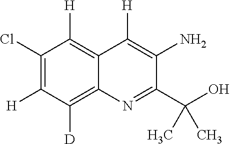 [SMILES: [2H]c1cc(Cl)cc2cc(N)c(C(C)(C)O)nc12] | X-5 |
| 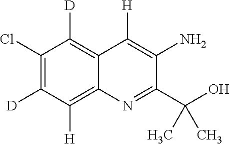 [SMILES: [2H]c1cc2nc(C(C)(C)O)c(N)cc2c([2H])c1Cl] | X-6 |
| 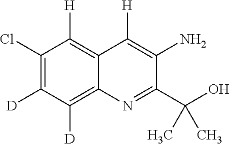 [SMILES: [2H]c1c(Cl)cc2cc(N)c(C(C)(C)O)nc2c1[2H]] | X-7 |
| 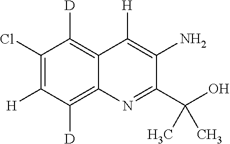 [SMILES: [2H]c1c(Cl)cc([2H])c2nc(C(C)(C)O)c(N)cc12] | X-8 |
| 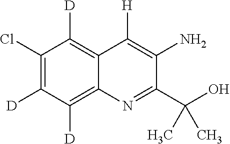 [SMILES: [2H]c1c(Cl)c([2H])c2cc(N)c(C(C)(C)O)nc2c1[2H]] | X-9 |
| 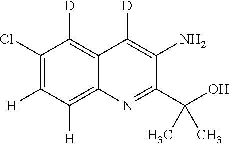 [SMILES: [2H]c1c(Cl)ccc2nc(C(C)(C)O)c(N)c([2H])c12] | X-10 |
| 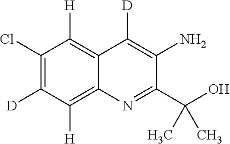 [SMILES: [2H]c1cc2nc(C(C)(C)O)c(N)c([2H])c2cc1Cl] | X-11 |
| 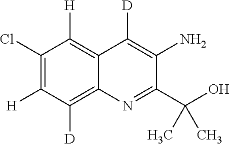 [SMILES: [2H]c1c(N)c(C(C)(C)O)nc2c([2H])cc(Cl)cc12] | X-12 |
| 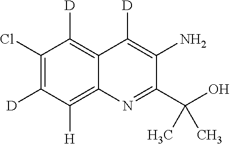 [SMILES: [2H]c1cc2nc(C(C)(C)O)c(N)c([2H])c2c([2H])c1Cl] | X-13 |
| 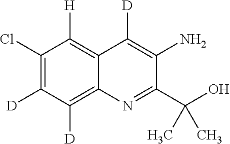 [SMILES: [2H]c1c(Cl)cc2c([2H])c(N)c(C(C)(C)O)nc2c1[2H]] | X-14 |
| 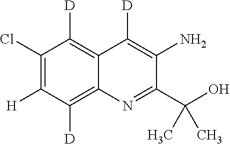 [SMILES: [2H]c1cc(Cl)c([2H])c2c([2H])c(N)c(C(C)(C)O)nc12] | X-15 |
| 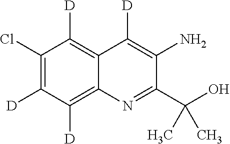 [SMILES: [2H]c1c(Cl)c([2H])c2c([2H])c(N)c(C(C)(C)O)nc2c1[2H]] | X-16 |
| 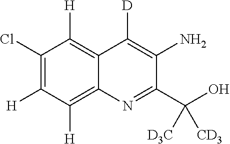 [SMILES: [2H]c1c(N)c(C(O)(C([2H])([2H])[2H])C([2H])([2H])[2H])nc2ccc(Cl)cc12] | X-17 |
| 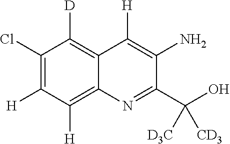 [SMILES: [2H]c1c(Cl)ccc2nc(C(O)(C([2H])([2H])[2H])C([2H])([2H])[2H])c(N)cc12] | X-18 |
| 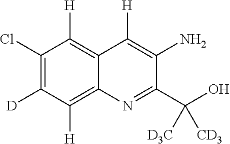 [SMILES: [2H]c1cc2nc(C(O)(C([2H])([2H])[2H])C([2H])([2H])[2H])c(N)cc2cc1Cl] | X-19 |
| 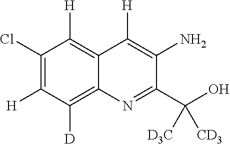 [SMILES: [2H]c1cc(Cl)cc2cc(N)c(C(O)(C([2H])([2H])[2H])C([2H])([2H])[2H])nc12] | X-20 |
| 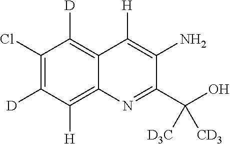 [SMILES: [2H]c1cc2nc(C(O)(C([2H])([2H])[2H])C([2H])([2H])[2H])c(N)cc2c([2H])c1Cl] | X-21 |
| 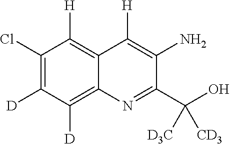 [SMILES: [2H]c1c(Cl)cc2cc(N)c(C(O)(C([2H])([2H])[2H])C([2H])([2H])[2H])nc2c1[2H]] | X-22 |
| 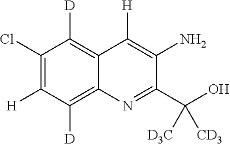 [SMILES: [2H]c1c(Cl)cc([2H])c2nc(C(O)(C([2H])([2H])[2H])C([2H])([2H])[2H])c(N)cc12] | X-23 |
| 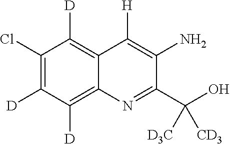 [SMILES: [2H]c1c(Cl)c([2H])c2cc(N)c(C(O)(C([2H])([2H])[2H])C([2H])([2H])[2H])nc2c1[2H]] | X-24 |
| 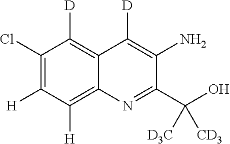 [SMILES: [2H]c1c(Cl)ccc2nc(C(O)(C([2H])([2H])[2H])C([2H])([2H])[2H])c(N)c([2H])c12] | X-25 |
| 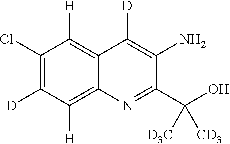 [SMILES: [2H]c1cc2nc(C(O)(C([2H])([2H])[2H])C([2H])([2H])[2H])c(N)c([2H])c2cc1Cl] | X-26 |
| 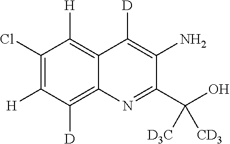 [SMILES: [2H]c1c(N)c(C(O)(C([2H])([2H])[2H])C([2H])([2H])[2H])nc2c([2H])cc(Cl)cc12] | X-27 |
| 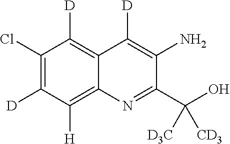 [SMILES: [2H]c1cc2nc(C(O)(C([2H])([2H])[2H])C([2H])([2H])[2H])c(N)c([2H])c2c([2H])c1Cl] | X-28 |
| 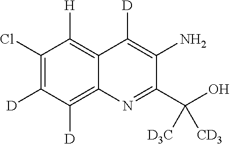 [SMILES: [2H]c1c(Cl)cc2c([2H])c(N)c(C(O)(C([2H])([2H])[2H])C([2H])([2H])[2H])nc2c1[2H]] | X-29 |
| 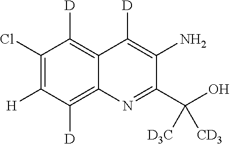 [SMILES: [2H]c1cc(Cl)c([2H])c2c([2H])c(N)c(C(O)(C([2H])([2H])[2H])C([2H])([2H])[2H])nc12] | X-30 |
| 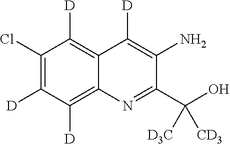 [SMILES: [2H]c1c(Cl)c([2H])c2c([2H])c(N)c(C(O)(C([2H])([2H])[2H])C([2H])([2H])[2H])nc2c1[2H]] | X-31 |

or a pharmaceutically acceptable salt thereof.

In some embodiments, the pharmaceutical composition comprises a compound depicted in Table 5, above, or a pharmaceutically acceptable salt thereof.

In some embodiments, the pharmaceutical composition comprises a deuterium-enriched analogue of a compound depicted in Table 6, below, or a pharmaceutically acceptable salt thereof, in which deuterium is enriched at any available hydrogen.

TABLE 6
Representative Compounds of Formula X

| | |
|---|---|
| 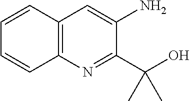 | X-32 |
| 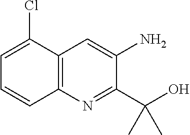 [SMILES: CC(C)(O)c1nc2cccc(Cl)c2cc1N] | X-33 |
| 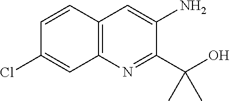 [SMILES: CC(C)(O)c1nc2cc(Cl)ccc2cc1N] | X-34 |
| 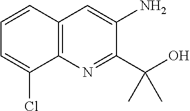 [SMILES: CC(C)(O)c1nc2c(Cl)cccc2cc1N] | X-35 |
| 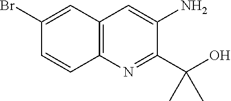 [SMILES: CC(C)(O)c1nc2ccc(Br)cc2cc1N] | X-36 |
| 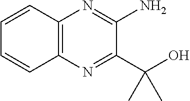 | X-37 |
| 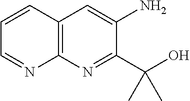 | X-38 |
| 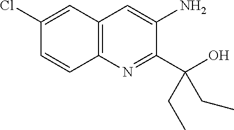 [SMILES: CCC(O)(CC)c1nc2ccc(Cl)cc2cc1N] | X-39 |
| 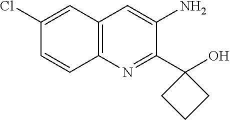 [SMILES: Nc1cc2cc(Cl)ccc2nc1C1(O)CCC1] | X-40 |
| 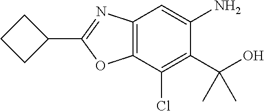 [SMILES: CC(C)(O)c1c(N)cc2nc(C3CCC3)oc2c1Cl] | X-41 |
| 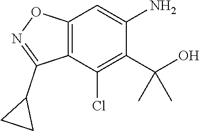 [SMILES: CC(C)(O)c1c(N)cc2onc(C3CC3)c2c1Cl] | X-42 |
| 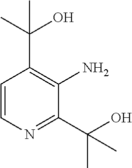 | X-43 |
| 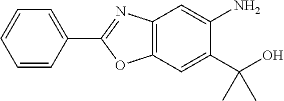 [SMILES: CC(C)(O)c1cc2oc(-c3ccccc3)nc2cc1N] | X-44 |
| 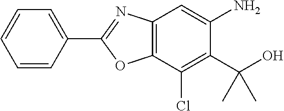 [SMILES: CC(C)(O)c1c(N)cc2nc(-c3ccccc3)oc2c1Cl] | X-45 |
| 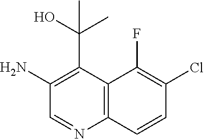 [SMILES: CC(C)(O)c1c(N)cnc2ccc(Cl)c(F)c12] | X-46 |
| 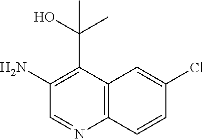 [SMILES: CC(C)(O)c1c(N)cnc2ccc(Cl)cc12] | X-47 |

In some embodiments, the compound is any compound described herein comprising one, two, three, four, five, six, seven, eight, nine, ten, eleven, twelve, or thirteen deuterium atoms.

In some embodiments, the compound is any compound described above and herein in isolated form. As used herein, the term “isolated” means that a compound is provide in s form that is separated from other compounds that might be present in that usual compounds environment. In some embodiments, an isolated compound is in solid form. In some embodiments, provided compounds comprise deuterium in an amount of about 5%, about 10%, about 15%, about 20%, about 25%, about 30%, about 35%, about 40%, about 45%, about 50%, about 55%, about 60%, about 65%, about 70%, about 75, about 80%, about 85%, about 90%, about 95%, or about 100%. As used herein in the context of deuterium enrichment, the term “about” means±2%.

### 4.4. Other Compounds

In some embodiments, the pharmaceutical composition comprises a compound of formula XX:

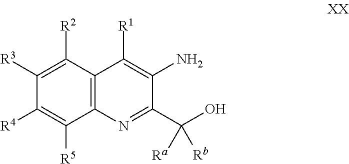

or a pharmaceutically acceptable salt thereof, wherein:

R¹ is H, D, or halogen;

R² is H, D, or halogen;

R³ is H, D, Br, or I;

R⁴ is H, D, or halogen;

R⁵ is H, D, or halogen;

R^{a} is C₁₋₄ aliphatic optionally substituted with 1, 2, or 3 deuterium or halogen atoms; and

R^{b} is C₁₋₄ aliphatic optionally substituted with 1, 2, or 3 deuterium or halogen atoms.

As defined generally above, R¹ is H, D, or halogen.

In some embodiments, R¹ is H. In some embodiments, R¹ is D. In some embodiments, R¹ is halogen. In some embodiments, R¹ is Cl. In some embodiments, R¹ is Br.

As defined generally above, R² is H, D, or halogen.

In some embodiments, R² is H. In some embodiments, R² is D. In some embodiments, R² is halogen. In some embodiments, R² is Cl. In some embodiments, R² is Br.

As defined generally above, R³ is H, D, Br, or I.

In some embodiments, R³ is H. In some embodiments, R³ is D. In some embodiments, R³ is Br. In some embodiments, R³ is I.

As defined generally above, R⁴ is H, D, or halogen.

In some embodiments, R⁴ is H. In some embodiments, R⁴ is D. In some embodiments, R⁴ is halogen. In some embodiments, R⁴ is Cl. In some embodiments, R⁴ is Br.

As defined generally above, R⁵ is H, D, or halogen.

In some embodiments, R⁵ is H. In some embodiments, R⁵ is D. In some embodiments, R⁵ is halogen. In some embodiments, R⁵ is Cl. In some embodiments, R⁵ is Br.

As defined generally above, R^{a} is C₁₋₄ aliphatic optionally substituted with 1, 2, or 3 deuterium or halogen atoms.

In some embodiments, R^{a} is C₁₋₄ aliphatic substituted with 1, 2, or 3 deuterium or halogen atoms. In some embodiments, R^{a} is C₁₋₄ aliphatic. In some embodiments, R^{a} is C₁₋₄alkyl. In some embodiments, R^{a} is methyl, ethyl, n-propyl, or isopropyl. In some embodiments, R^{a} is methyl.

As defined generally above, R^{b} is C₁₋₄ aliphatic optionally substituted with 1, 2, or 3 deuterium or halogen atoms.

In some embodiments, R^{b} is C₁₋₄ aliphatic substituted with 1, 2, or 3 deuterium or halogen atoms. In some embodiments, R^{b} is C₁₋₄ aliphatic. In some embodiments, R^{b} is C₁₋₄ alkyl. In some embodiments, R^{b} is C₁₋₄ alkyl optionally substituted with 1, 2, or 3 fluorine atoms. In some embodiments, R^{b} is methyl, ethyl, n-propyl, or isopropyl. In some embodiments, R^{b} is methyl.

In some embodiments, R^{a} and R^{b} are methyl or ethyl. In some embodiments, R^{a} and R^{b} are methyl.

In some embodiments, the compound is a compound of formula XX-a:

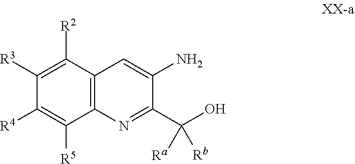

or a pharmaceutically acceptable salt thereof, wherein:
each of R², R³, R⁴, R⁵, R^{a}, and R^{b} is as defined above and described in embodiments herein, both singly and in combination.

In some embodiments, the compound is a compound of formula XX-b:

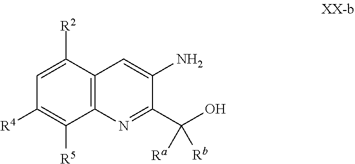

or a pharmaceutically acceptable salt thereof, wherein:
each of R², R⁴, R⁵, R^{a}, and R^{b} is as defined above and described in embodiments herein, both singly and in combination.

In some embodiments, the compound is a compound of formula XX-c, XX-d, XX-e, or XX-f:

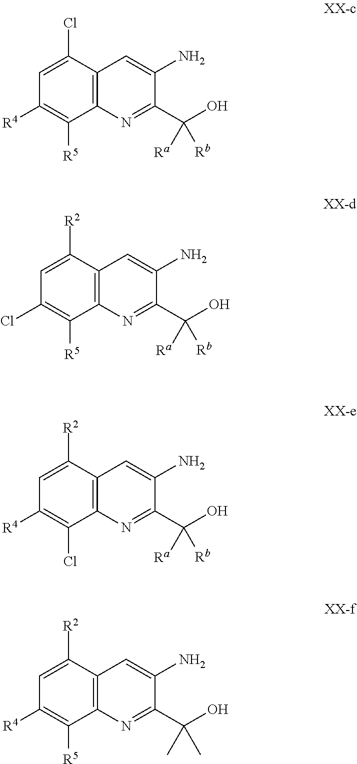

or a pharmaceutically acceptable salt thereof, wherein:

each of R², R⁴, R⁵, R^{a}, and R^{b} is as defined above and described in embodiments herein, both singly and in combination.

In some embodiments, the compound is a compound of formula XX-g, XX-h, XX-i, or XX-j:

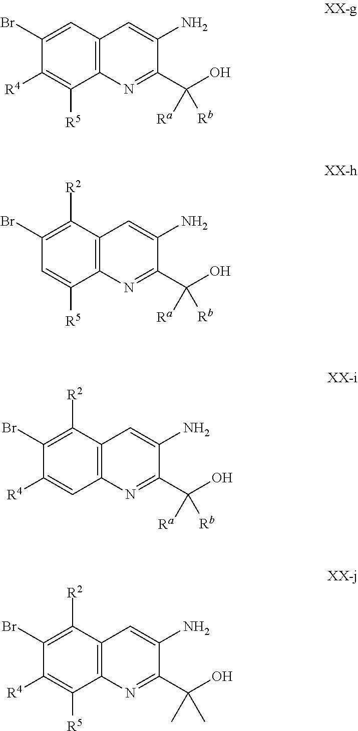

or a pharmaceutically acceptable salt thereof, wherein:
each of R², R⁴, R⁵, R^{a}, and R^{b} is as defined above and described in embodiments herein, both singly and in combination.

In some embodiments, the compound is a compound of formula XX-k or XX-l:

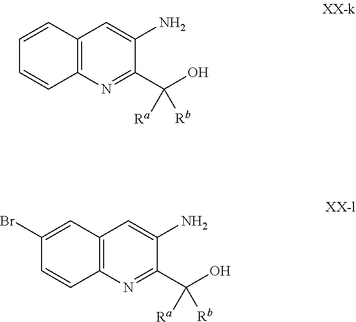

or a pharmaceutically acceptable salt thereof, wherein:
each of R^{a} and R^{b} is as defined above and described in embodiments herein, both singly and in combination.

In some embodiments, the pharmaceutical composition comprises a compound of formula I-5:

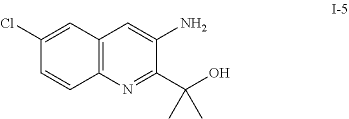 [SMILES: CC(C)(O)c1nc2ccc(Cl)cc2cc1N]

or a pharmaceutically acceptable salt thereof, in combination with at least one compound of formula XX:

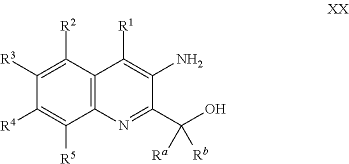

or a pharmaceutically acceptable salt thereof, wherein:

R¹ is H, D, or halogen;

R² is H, D, or halogen;

R³ is H, D, Br, or I;

R⁴ is H, D, or halogen;

R⁵ is H, D, or halogen;

R^{a} is C₁₋₄ aliphatic optionally substituted with 1, 2, or 3 deuterium or halogen atoms; and

R^{b} is C₁₋₄ aliphatic optionally substituted with 1, 2, or 3 deuterium or halogen atoms.

In some embodiments, the pharmaceutical composition comprises a compound of formula I-5, or a pharmaceutically acceptable salt thereof, and at least one compound according to formula XX-a, XX-b, XX-c, XX-d, XX-e, XX-f, XX-g, XX-h, XX-i, XX-j, XX-k, or XX-l; or a pharmaceutically acceptable salt thereof.

In some embodiments, the pharmaceutical composition comprises a compound of formula I-5:

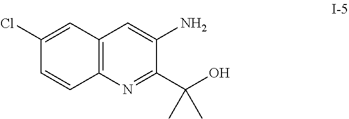 [SMILES: CC(C)(O)c1nc2ccc(Cl)cc2cc1N]

or a pharmaceutically acceptable salt thereof, and a compound selected from the following, or a pharmaceutically acceptable salt thereof:

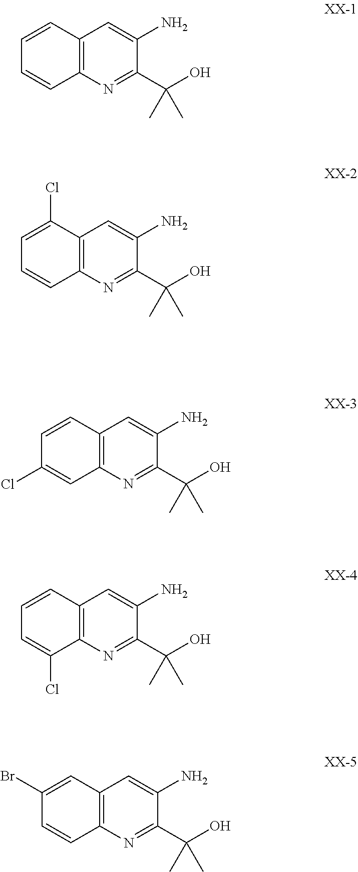 [SMILES: CC(C)(O)c1nc2ccccc2cc1N ; CC(C)(O)c1nc2cccc(Cl)c2cc1N ; CC(C)(O)c1nc2cc(Cl)ccc2cc1N ; CC(C)(O)c1nc2c(Cl)cccc2cc1N ; CC(C)(O)c1nc2ccc(Br)cc2cc1N]

In some embodiments, the pharmaceutical composition comprises a compound of formula I-5, or a pharmaceutically acceptable salt thereof, and one additional compound selected from XX-1, XX-2, XX-3, XX-4, and XX-5; or a pharmaceutically acceptable salt thereof.

In some embodiments, the pharmaceutical composition comprises a compound of formula I-5, or a pharmaceutically acceptable salt thereof, and two additional compounds selected from XX-1, XX-2, XX-3, XX-4, and XX-5; or a pharmaceutically acceptable salt thereof.

In some embodiments, the pharmaceutical composition comprises a compound of formula I-5, or a pharmaceutically acceptable salt thereof, and three additional compounds selected from XX-1, XX-2, XX-3, XX-4, and XX-5; or a pharmaceutically acceptable salt thereof.

In some embodiments, the pharmaceutical composition comprises a compound of formula I-5, or a pharmaceutically acceptable salt thereof, and four additional compounds selected from XX-1, XX-2, XX-3, XX-4, and XX-5; or a pharmaceutically acceptable salt thereof.

In some embodiments, the pharmaceutical composition comprises a compound of formula I-5, or a pharmaceutically acceptable salt thereof, and one additional compound selected from XX-2, XX-3, and XX-4; or a pharmaceutically acceptable salt thereof.

In some embodiments, the pharmaceutical composition comprises a compound of formula I-5, or a pharmaceutically acceptable salt thereof, and two additional compounds selected from XX-2, XX-3, and XX-4; or a pharmaceutically acceptable salt thereof. In some embodiments, the composition comprises XX-2, XX-3, and XX-4; or a pharmaceutically acceptable salt thereof.

In some embodiments, the pharmaceutical composition comprises a compound of formula I-5, or a pharmaceutically acceptable salt thereof, and XX-1 or a pharmaceutically acceptable salt thereof.

In some embodiments, the pharmaceutical composition comprises a compound of formula I-5, or a pharmaceutically acceptable salt thereof, and XX-2; or a pharmaceutically acceptable salt thereof.

In some embodiments, the pharmaceutical composition comprises a compound of formula I-5, or a pharmaceutically acceptable salt thereof, and XX-3; or a pharmaceutically acceptable salt thereof.

In some embodiments, the pharmaceutical composition comprises a compound of formula I-5, or a pharmaceutically acceptable salt thereof, and XX-4; or a pharmaceutically acceptable salt thereof.

In some embodiments, the pharmaceutical composition comprises a compound of formula I-5, or a pharmaceutically acceptable salt thereof, and XX-5; or a pharmaceutically acceptable salt thereof.

In some embodiments, the pharmaceutical composition comprises a compound of formula XX selected from those depicted in Table 7, below.

TABLE 7
Representative Compounds of Formula XX

| | |
|---|---|
| 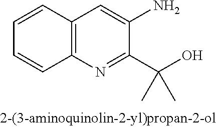 | XX-1 |
| 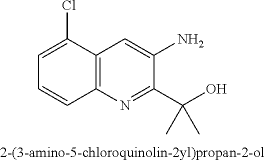 [SMILES: CC(C)(O)c1nc2cccc(Cl)c2cc1N] | XX-2 |
| 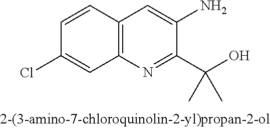 [SMILES: CC(C)(O)c1nc2cc(Cl)ccc2cc1N] | XX-3 |
| 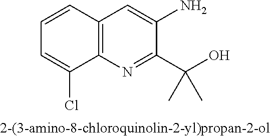 [SMILES: CC(C)(O)c1nc2c(Cl)cccc2cc1N] | XX-4 |
| 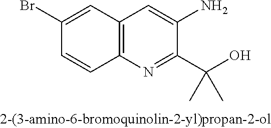 [SMILES: CC(C)(O)c1nc2ccc(Br)cc2cc1N] | XX-5 |

In some embodiments, the pharmaceutical composition comprises a compound depicted in Table 7, above, or a pharmaceutically acceptable salt thereof.

In some embodiments, the compound for use in the pharmaceutical composition is any compound described above and herein in isolated form. As used herein, the term “isolated” means that a compound is provided in a form that is separated from other components that might be present in that compound's usual environment. In some embodiments, an isolated compound is in solid form. In some embodiments, an isolated compound is at least about 50% pure as determined by a suitable HPLC method. In certain embodiments, an isolated compound is at least about 60%, 70%, 80%, 90%, 95%, 98%, 99%, 99.5%, 99.6%, 99.7%, 99.8%, 99.9%, 99.95%, 99.99%, or 99.999% as determined by a suitable HPLC method. Methods of preparation applicable to certain compounds of the invention are disclosed in US 2013/0190500, published Jul. 25, 2013, which is hereby incorporated by reference.

In some embodiments, the pharmaceutical composition comprises a compound of any one of formula XX, XX-a, XX-b, XX-c, XX-d, XX-e, XX-f, XX-g, XX-h, XX-i, XX-j, XX-k, or XX-l, or a pharmaceutically acceptable salt thereof, in an amount of at least about 97, 97.5, 98, 98.5, 99.0, 99.5, 99.8, 99.9, 99.95, or 99.999 weight percent where the percentages are based on the free base of said compound and the total weight of the composition. In other embodiments, the composition contains no more than about 2.0 area percent HPLC of total organic impurities or, in other embodiments, no more than about 1.5, 1.25, 1, 0.75, 0.5, 0.25, 0.2, 0.1, 0.01, 0.005, or 0.001 area percent HPLC total organic impurities relative to the total area of the HPLC chromatogram.

In other embodiments, the pharmaceutical composition comprises a compound of formula I-5 or a pharmaceutically acceptable salt thereof, at least one compound of formula XX, XX-a, XX-b, XX-c, XX-d, XX-e, XX-f, XX-g, XX-h, XX-i, XX-j, XX-k, or XX-l, or a pharmaceutically acceptable salt thereof, and at least one pharmaceutically acceptable carrier. In some embodiments, the composition contains the compound of formula I-5 or pharmaceutically acceptable salt thereof in an amount of about 1 weight percent to about 99 weight percent, where the percentages are based on the free base of said compound and on the total weight of the composition. In other embodiments, the composition contains no more than about 2.0 area percent HPLC of total organic impurities or, in other embodiments, no more than about 1.5, 1.25, 1, 0.75, 0.5, 0.25, 0.2, 0.1, 0.01, 0.005, or 0.001 area percent HPLC total organic impurities relative to the total area of the HPLC chromatogram.

In some embodiments, the pharmaceutical composition comprises a compound of formula I-5 or pharmaceutically acceptable salt thereof and a compound of formula XX, X-a, XX-b, XX-c, XX-d, XX-e, XX-f, XX-g, XX-h, XX-i, XX-j, XX-k, or XX-l, or pharmaceutically acceptable salt thereof, wherein the compound of formula I-5 or pharmaceutically acceptable salt thereof comprises about 98% and the compound of formula XX, XX-a, XX-b, XX-c, XX-d, XX-e, XX-f, XX-g, XX-h, XX-i, XX-j, XX-k, or XX-l, or pharmaceutically acceptable salt thereof comprises about 2% of the total weight of the compounds or pharmaceutically acceptable salts thereof taken together or of the total HPLC peak area of the compounds or pharmaceutically acceptable salts thereof taken together. In some embodiments, the pharmaceutical composition comprises a compound of formula I-5 or pharmaceutically acceptable salt thereof and a compound of formula XX, XX-a, XX-b, XX-c, XX-d, XX-e, XX-f, XX-g, XX-h, XX-i, XX-j, XX-k, or XX-l, or pharmaceutically acceptable salt thereof, wherein the compound of formula I-5 or pharmaceutically acceptable salt thereof comprises about 99%, 99.5%, 99.6%, 99.7%, 99.8%, 99.9%, 99.95%, 99.99%, or 99.999%, and the compound of formula XX, XX-a, XX-b, XX-c, XX-d, XX-e, XX-f, XX-g, XX-h, XX-i, XX-j, XX-k, or XX-l, or pharmaceutically acceptable salt thereof comprises about 1%, 0.5%, 0.4%, 0.3%, 02%, 0.1%, 0.05%, 0.01%, or 0.001%, of the total weight of the compounds or pharmaceutically acceptable salts thereof taken together or of the total HPLC peak area of the compounds or pharmaceutically acceptable salts thereof taken together. In some embodiments, the compound of formula XX, XX-a, XX-b, XX-c, XX-d, XX-e, XX-f, XX-g, XX-h, XX-i, XX-j, XX-k, or XX-l, or pharmaceutically acceptable salt thereof comprises about 100 ppm, 50 ppm, 10 ppm, 1 ppm, 500 ppb, 100 ppb, or 10 ppb of the total weight of the compounds or pharmaceutically acceptable salts thereof taken together.

In some embodiments, the pharmaceutical composition comprises a compound of formula I-5 or pharmaceutically acceptable salt thereof and a compound of formula XX, XX-a, XX-b, XX-c, XX-d, XX-e, Xx-f XX-g, XX-h, XX-i, XX-j, XX-k, or XX-l, or pharmaceutically acceptable salt thereof, wherein the compound of formula I-5 or pharmaceutically acceptable salt thereof comprises about 99%-99.9999%, 99.5-99.9999%, 99.6-99.9999%, 99.7-99.9999%, 99.8-99.9999%, 99.9-99.9999%, 99.95-99.9999%, 99.99-99.9999%, or 99.999-99.9999%, and the compound of formula XX, XX-a, XX-b, XX-c, XX-d, XX-e, XX-f, XX-g, XX-h, XX-i, XX-j, XX-k, or XX-l, or pharmaceutically acceptable salt thereof comprises about 10 ppm to 2%, 100 ppm to 1%, 0.0001-0.5%, 0.0001-0.4%, 0.0001-0.3%, 0.0001-0.2%, 0.0001-0.1%, 0.0001-0.05%, 0.0001-0.01%, or 0.0001-0.001% of the total weight of the compounds or pharmaceutically acceptable salts thereof taken together.

In some embodiments, the compound of formula I-5 or pharmaceutically acceptable salt thereof and the compound of formula XX, XX-a, XX-b, XX-c, XX-d, XX-e, XX-f, XX-g, XX-h, XX-i, XX-j, XX-k, or XX-l, or pharmaceutically acceptable salt thereof, are present in a ratio of about 98:2, 99:1, 99.5:0.5, 99.6:0.4, 99.7:0.3, 99.8:0.2, 99.9:0.1, 99.95:0.05, 99.99:0.01, or 99.999:0.001.

In some embodiments, the compound of any of formula XX, XX-a, XX-b, XX-c, XX-d, XX-e, XX-f, XX-g, XX-h, XX-i, XX-j, XX-k, or XX-l, or pharmaceutically acceptable salt thereof, comprises about 0.01-0.20 area percent of the HPLC chromatogram relative to the compound of formula I-5 or pharmaceutically acceptable salt thereof. In some embodiments, the compound of formula XX, XX-a, XX-b, XX-c, XX-d, XX-e, XX-f, XX-g, XX-h, XX-i, XX-j, XX-k, or XX-l, or pharmaceutically acceptable salt thereof, comprises about 0.02-0.18, 0.03-0.16, 0.05-0.15, 0.075-0.13, 0.09-0.1, 0.1-0.2, or 0.15-0.2 area percent of the HPLC chromatogram relative to the compound of formula I-5 or pharmaceutically acceptable salt thereof. In some embodiments, the foregoing area percentages of the HPLC chromatogram are measured relative to the total area of the HPLC chromatogram.

The compounds for the pharmaceutical compositions can be synthesized using processes and schemes available to the skilled artisan. Synthetic schemes and processes are described in, for example, WO2006127945, WO2014100425, WO2014116593, WO2017035077, WO2017035082, WO2018039192, and WO2018039197; all references incorporated herein by reference.

In the pharmaceutical compositions, the amount of the compound in the pharmaceutical composition is an amount sufficient to provide a therapeutically effective amount when administered to a subject. In some embodiments, the compound, as part of the pharmaceutical composition, can comprise about 0.01% w/v, 0.02% w/v, 0.05% w/v, 0.07% w/v, 0.1% w/v, 0.15% w/v, 0.2% w/v, 0.3% w/v, 0.4% w/v, 0.5% w/v, 0.6% w/v, 0.7% w/v, 0.8% w/v, 0.9% w/v, 1% w/v, 1.5% w/v, 2% w/v, 3% w/v, 4% w/v, 5% w/v, 6% w/v, 7% w/v, 8% w/v, 9% w/v or 10% w/v.

In some embodiments, the amount of compound in the pharmaceutical composition is about 0.01% w/v to about 10% w/v, about 0.02% w/v to about 10% w/v, about 0.05% w/v to about 10% w/v, about 0.07% w/v to about 10% w/v, about 0.1% w/v to about 10% w/v, about 0.15% w/v to about 10% w/v, about 0.2% w/v to about 10% w/v, about 0.3% w/v to about 10% w/v, about 0.4% w/v to about 10% w/v, about 0.5% w/v to about 10% w/v, about 0.6% w/v to about 10% w/v, about 0.7% w/v to about 10% w/v, about 0.8% w/v to about 10% w/v, about 0.9% w/v to about 10% w/v, about 1% w/v to about 10% w/v, about 1.5% w/v to about 10% w/v, about 2% w/v to about 10% w/v, about 3% w/v to about 10% w/v, about 4% w/v to about 10% w/v, about 5% w/v to about 10% w/v, about 6% w/v to about 10% w/v, about 7% w/v to about 10% w/v, about 8% w/v to about 10% w/v.

In some embodiments, the amount of compound in the pharmaceutical composition is about 0.01% w/v to about 10% w/v, about 0.02% w/v to about 9% w/v, about 0.05% w/v to about 8% w/v, about 0.07% w/v to about 7% w/v, about 0.1% w/v to about 6% w/v, about 0.2% w/v to about 5% w/v, about 0.3% w/v to about 4% w/v, about 0.4% w/v to about 3% w/v, or about 0.5% w/v to about 2% w/v.

### 4.5. Viscosity Enhancing Agents and Hyaluronate Formulations

In some embodiments, the pharmaceutical composition comprises any of the compounds above and a viscosity enhancing agent. In some embodiments, the pharmaceutical composition has a viscosity sufficient to reduce substantial diffusion of the therapeutic agent contained therein from the site at which the composition is administered and/or to provide delayed release of the compound. A suitable viscosity enhancing agent is selected from, among others, hyaluronate, hyaluronic acid or pharmaceutically acceptable salt thereof; cross-linked hyaluronic acid; polyvinylpyrrolidone (PVP); hydroxypropyl methylcellulose, hydroxypropyl cellulose, hydroxylethyl cellulose, glycerol; and mixtures thereof. In some embodiments, a viscosity enhancing agent is selected that does not have reactive aldehyde groups capable of reacting with the compounds described herein.

In some embodiments, the viscosity enhancing agent is sodium hyaluronate, polyvinylpyrrolidone (PVP), sodium hydroxypropyl cellulose, or hydroxypropyl methylcellulose. The viscosity enhancing component is present in an amount effective in providing the desired viscosity to the composition.

In some embodiments, the amount of the viscosity enhancing agent is based on the agent used, and is in general in an amount of about 0.05 to 30% w/v. In some embodiments, the concentration of viscosity enhancing agent is about 0.05 to 30% w/v, about 0.1% w/v to about 25% w/v, about 0.25% w/v to about 15% w/v, about 0.5% w/v to about 15% w/v, about 0.75% w/v to about 10% w/v, or about 1.0% w/v to about 5% w/v.

In some embodiments, the amount of the viscosity enhancing agent in the pharmaceutical composition is 0.05% w/v to 1.5% w/v; 0.05% w/v to 0.5% w/v; 0.1% w/v to 3.0% w/v; 0.1% w/v to 1.5% w/v; 0.1% w/v to 1.0% w/v; 0.5% w/v to 1% w/v; 0.5% w/v to 2.5% w/v; 1.0% w/v to 3.0% w/v; 1.0% w/v to 1.5% w/v; 1.0% w/v to 1.25% w/v; 1.25% w/v to 1.5% w/v; or 1.5% w/v to 3.0% w/v.

In some embodiments, the amount of the viscosity enhancing agent in the pharmaceutical composition is about 0.1% w/v, about 0.25% w/v, about 0.5% w/v, about 0.75% w/v, about 1.0% w/v, about 1.1% w/v, about 1.15% w/v, about 1.20% w/v, about 1.25% w/v, about 1.30% w/v, about 1.35% w/v, about 1.40% w/v, about 1.45% w/v, about 1.5% w/v, about 2% w/v, about 3% w/v, about 4% w/v, about 5% w/v, about 10% w/v, about 15% w/v, about 20% w/v, about 25% w/v, or about 30% w/v.

In some embodiments, the molecular weight of a viscosity enhancing agent when polymeric is about 500,000 to about 5×10⁶ Daltons; about 500,000 Daltons to about 3×10⁶ Daltons; about 500,000 to about 2×10 Daltons; about 500,000 to about 1×10⁶ Daltons; about 500,000 to about 2×106 Daltons; about 1×10⁶ Daltons to about 3×10⁶ Daltons; about 1×10⁶ Daltons to about 2.5×10⁶ Daltons; about 1×10⁶ Daltons to about 2×10⁶ Daltons; or about 1.2×10⁶ Daltons to about 1.8×10⁶ Daltons. In some embodiments, the molecular weight is the number average molecular weight, and in other embodiments, the molecular weight is the weight average molecular weight.

In some embodiments, the viscosity of the pharmaceutical composition is about 300 kcP, about 250 kcP, about 200 kcP, about 150 kcP, about 140 kcP, about 130 kcP, about 120 kcP, about 110 kcP, about 100 kcP, about 90 kcP, about 80 kcP, about, 70 kcP, about 40 kcP, about, 30 kcP, about 25 kcP, about 20 kcP, about 10 kcP, about 5 kcP, or about 1 kcP.

In some embodiments, the viscosity of the composition is about 1 kcP to about 300 kcP; about 1 kcP to about 200 kcP, about 1 kcP to about 100 kcP; about 1 kcP to about 50 kcP; about 1 kcP to about 10 kcP; about 10 kcP to about 50 kcP; about 10 kcP to about 100 kcP; about 50 kcP to about 100 kcP; about 100 kcP to about 300 kcP; about 50 kcP to about 200 kcP; about 75 kcP to about 180 kcP; about 100 kcP to about 150 kcP; about 150 kcP to about 200 kcP; about 200 kcP to about 250 kcP; or about 250 kcP to about 300 kcP.

In some embodiments, the pharmaceutical composition comprises a compound of the present disclosure and hyaluronic acid or a pharmaceutically acceptable salt of the hyaluronic acid. Hyaluronic acid is a glycosaminoglycan, whose molecular weight can vary from 50,000 Daltons to about 8,000,000 Daltons and forms highly viscous solutions. In some embodiments, the hyaluronic acid contained in the formulation can be, either in its acid form or in the form of one of its pharmaceutically acceptable salts, such as an alkali metal or alkaline-earth metal hyaluronate, for instance sodium hyaluronate, potassium hyaluronate, magnesium hyaluronate, calcium hyaluronate or others. In some embodiments, the hyaluronic acid is in the form of sodium hyaluronate. In some embodiments, the hyaluronate is sodium hyaluronate solution in combination with a citric acid salt, for example tri-sodium citrate.

In some embodiments of the pharmaceutical compositions, the hyaluronate is a high molecular weight (HMW) hyaluronic acid or a pharmaceutically acceptable salt thereof. As used herein, HMW hyaluronic acid refers to a hyaluronic acid material having a molecular weight of at least about 1×10⁶ to about 5×10⁶ Daltons. In some embodiments, the HMW hyaluronic acid is 1×10⁶ to about 5×10⁶ Daltons; 1.5×10⁶ to about 4×10⁶ Daltons, 2×10⁶ to about 4×10⁶ Daltons; 2×10⁶ to about 4×10⁶ Daltons; 1×10⁶ to about 3×10⁶ Daltons; or 1.5×10⁶ to about 2×10⁶ Daltons. In some embodiments, the HMW hyaluronic acid in the pharmaceutical compositions can have a molecular weight of about 1×10⁶ Daltons. In some embodiments, the HMW hyaluronic acid can have a molecular weight of about 2.8×10⁶ Daltons. In some embodiments, the hyaluronic acid or its salt has a weight-average molecular weight that is not below 1×10⁶ Daltons, more preferably an average molecular weight in the range of 1.3×10⁶ to 3×10⁶ Daltons. In some embodiments, the molecular weight is about 1.7×10⁶ Daltons.

In some embodiments, the hyaluronate is a low molecular weight (LMW) hyaluronic acid or a pharmaceutically acceptable salt thereof. As used herein, a LMW hyaluronic acid refers to hyaluronic acid material having a molecular weight of less than about 1×10⁶ Daltons. In some embodiments, the LMW hyaluronic acid can have a molecular weight of between about 200,000 to less than about 1×10⁶ Daltons, for example, between about 300,000 to about 750,000 Daltons. In some embodiments, an average molecular weight is less than about 750,000. In some embodiments, the average molecular weight of the hyaluronate component is in a range of about 50,000 or about 100,000 Daltons to about 750,000 Daltons.

In some embodiments, an average molecular weight of the hyaluronic acid or salt thereof is in a range of about 10,000 Daltons or less to about 2×10⁶ Daltons. In some embodiments, the average molecular weight of the hyaluronic acid is in a range of about 100,000 Daltons or about 200,000 Daltons to about 1×10⁶ Daltons, or to about 1×10⁶ Daltons. In some embodiments, the molecular weight of the hyaluronic acid component can be varied over a substantial range to obtain the desired final viscosity of the composition. In some embodiments, two or more distinct molecular weight ranges of the hyaluronic acid may be used to increase the shear thinning attributes of the composition. In some embodiments, the hyaluronate is a metal hyaluronate component, preferably selected from alkali metal hyaluronates, alkaline earth metal hyaluronates and mixtures thereof, and still more preferably selected from sodium or potassium hyaluronates, and mixtures thereof.

In some embodiments, the molecular weight of the hyaluronate or hyaluronic acid or a pharmaceutically acceptable salt thereof is about 500,000 to about 5×10⁶ Daltons; about 500,000 to about 3×10⁶ Daltons; about 500,000 to about 2×10⁶ Daltons; about 500,000 to about 1×10⁶ Daltons; about 500,000 to about 2×10⁶ Daltons; about 1×10⁶ to about 3×10⁶ Daltons; about 1×10⁶ to about 2.5×10⁶ Daltons; about 1×10⁶ to about 2×10⁶ Daltons; or about 1.2×10⁶ to about 1.8×10⁶ Daltons.

In some embodiments, the amount of hyaluronate in the pharmaceutical composition is about 0.05% w/v; about 0.1% w/v, about 0.25% w/v, about 0.5% w/v, about 0.75% w/v, about 1.0% w/v, about 1.1% w/v, about 1.15% w/v, about 1.20% w/v, about 1.25% w/v, about 1.30% w/v, about 1.35% w/v, about 1.40% w/v, about 1.45% w/v, about 1.5% w/v; about 2% w/v; about 2.5% w/v; about 3% w/v; about 3.5% w/v; or about 4% w/v.

In some embodiments, the amount of hyaluronate in the pharmaceutical composition is about 0.05% w/v to about 1.5% w/v; about 0.05% w/v to about 0.5% w/v; about 0.1% w/v to about 4.0% w/v; about 0.1% w/v to about 3.0% w/v; about 0.1% w/v to about 1.5% w/v; about 0.1% w/v to about 1.0% w/v; about 0.5% w/v to about 1% w/v; about 0.5% w/v to about 2.5% w/v; about 1.0% w/v to about 3.0% w/v; about 1.0% w/v to about 1.5% w/v; about 1.0% w/v to about 1.25% w/v; about 1.25% w/v to about 1.5% w/v; or about 1.5% w/v to about 3.0% w/v.

In some embodiments, the hyaluronate is in an amount in a range about 0.05% to about 0.5% (w/v). In some embodiments, the hyaluronate is present in an amount in a range of about 1% to about 4% (w/v). At the higher concentrations, the high viscosity can result in a gel that slows particle sedimentation and diffusion of dissolved solutes, such as upon injection into the eye. Such a composition can be provided in pre-filled syringes for ease of administration.

In some embodiments, the hyaluronic acid is a crosslinked hyaluronic acid. In some embodiments, the hyaluronic acid is present at about 50% to 99% by weight, or 70% to 95% by weight in the form of a crosslinked hyaluronic acid, from 1% to 50% by weight, preferably 5% to 30% by weight, of hyaluronic acid present in the free form or a pharmaceutically acceptable salt thereof. In some embodiments where the hyaluronic acid is crosslinked, the crosslinked hyaluronic acid can have a degree of modification ranging from 0.1 to 20%, preferably from 0.4 to 10%. In some embodiments, the cohesive, crosslinked hyaluronic-based gel includes no greater than about 1% to about 10% of free hyaluronic acid material by volume, for example, no greater than about 5% free hyaluronic acid material.

In some embodiments, the cross-linked hyaluronic acid can be prepared by, among others, use of dihydrazides, photocrosslinking, enzymatic cross-linking, expoxidic crosslinks, and click chemistry. The “degree of modification” refers to the ratio between the number of moles of crosslinking agent attached to the hyaluronic acid and the number of moles of hyaluronic acid forming said crosslinked hyaluronic acid gel. Free hyaluronic acid is generally water soluble, and as used herein, can be defined as the “uncrosslinked,” or lightly crosslinked component of the macromolecular structure. Crosslinked hyaluronic acid and methods of their preparation are described in, among others, Schramm et al., 2012, Invest Ophthalmol Vis Sci. 53:613-621; Schramm et al., 2011, Invest Ophthalmol Vis Sci. 52:452; Egbu et al., 2018, Eur J Pharm Biopharm. 124:95-103; U.S. Pat. Nos. 8,357,795; 9,925,309; all of which are incorporated herein by reference in their entireties.

In some embodiments, the viscosity enhancing agent is hyaluronic acid crosslinked with dextran (see, e.g., Yu et al., 2015, Transl Vis Sci Technol. 4(2):5; incorporated herein by reference). Synthesis of hyaluronic acid crosslinked with dextran can be carried out by chemical crosslinking between functionalized hyaluronic acid and thiolated dextran. In some embodiments, the viscosity enhancing agent is a copolymer of hyaluronic acid and poly(glyceryl glycerol) (PGG) side chains attached via hydrolysable ester linkers (see, e.g., Borke et al., 2018, Macromolecular Bioscience 18:1700200; incorporated herein by reference).

In some embodiments, for intraocular, e.g., intravitreal, formulations, the intraocular dosage composition of hyaluronate has a viscosity sufficient to reduce substantial diffusion of the drug contained therein from the site at which the composition is administered. In some embodiments, the pharmaceutical compositions have viscosity of at least about 10 cps or at least about 100 cps or at least about 1000 cps, more preferably at least about 10,000 cps and still more preferably at least about 70,000 cps or more, for example up to about 200,000 eps or about 250,000 cps, or about 300,000 cps or more, at a shear rate of 0.1/second. In some embodiments, the composition has sufficient viscosity to be structured or formed but injectable into a posterior segment of an eye of a human or animal, for example through a 27 gauge needle, or even through a 30 gauge needle.

In some embodiments, the pharmaceutical composition comprising a compound disclosed herein and a viscosity enhancing agent, further comprises one or more excipients. In some embodiments, the excipient is one or more of tonicity agent, buffering agent, chelating agent, surfactant, preservative, antioxidant, and an additional viscosity enhancing agent different from the first viscosity enhancing agent.

In some embodiments, the pharmaceutical composition further comprises a tonicity agent. In some embodiments, the pharmaceutical composition further comprises a buffering agent. In some embodiments, the pharmaceutical composition further comprises a chelating agent. In some embodiments, the pharmaceutical composition further comprises a surfactant. In some embodiments, the pharmaceutical composition further comprises a preservative. In some embodiments, the pharmaceutical composition further comprises an antioxidant. In some embodiments, the pharmaceutical composition further comprises an additional viscosity enhancing agent different from the first viscosity enhancing agent. In some embodiments, the excipients are suitable for intraocular, e.g., intravitreal, administration.

### 4.6. Liposomal Formulations

In some embodiments, a pharmaceutical composition comprises a compound of the disclosure formulated in a liposome. Liposomes are artificial, self-closed vesicular structures of various sizes and structures, where one or several membranes encapsulate an aqueous core. In some embodiments, liposome compositions are selected that do not have aldehyde groups capable of reacting with the compounds described herein.

In some embodiments, the types of liposomes include, multilamellar vesicles (MLV) or oligolamellar vesicles (OLV), unilamellar vesicles (SUV) and large unilamellar vesicles (LUV). Generally, a unilamellar liposome is a liposome having a single bilayer of an amphiphilic lipid or a mixture of such lipids, containing aqueous solution inside the chamber. Generally, MLVs have more than one lipid bilayer, where the lipid bilayers in MLVs are separated from the one another other by an aqueous solution.

In some embodiments, small unilamellar liposomes/vesicles (SUVs) have diameters up to 100 nm, for example 20 um to about 100 um; large unilamellar liposomes/vesicles (LUVs) can have diameters greater than 100 nm, and up to micrometers (μm), the latter sometimes referred to as giant unilamellar vesicles (GUV). MLVs can have diameters of 200 nm to 3 um, for example greater than 0.5 um. In some embodiments, the diameter is a mean diameter. As used herein, “mean diameter” refers generally to a mathematical average of a set of diameters, each diameter being taken for each liposome in a liposome population.

In some embodiments, the pharmaceutical composition comprises a compound of the disclosure formulated in a liposome, preferably as small unilamellar vesicles (SUV) or large unilamellar vesicles (LUV) or multilamellar vesicles (MLV). In some embodiments, the liposomes have a mean diameter of less than about 1 um (i.e., submicron-sized liposomes).

In some embodiments, the liposomes have a mean diameter of about 20 nm to about 1 um or less; about 20 nm to about 900 nm; about 20 nm to about 800 nm; about 20 nm to about 700 nm, about 20 nm to about 600 nm; about 20 nm to about 500 nm; about 20 nm to about 450 nm; about 20 nm to about 400 nm; about 20 nm to about 350 nm; about 20 nm to about 300 nm; about 20 nm to about 250 nm; about 20 nm to about 200 nm; about 20 nm to about 150 nm; about 20 nm to about 125 nm; about 20 nm to about 100 nm; about 20 nm to about 80 nm; about 20 nm to about 70 nm; or; about 20 nm to about 50 nm.

In some embodiments, the liposomes have a mean diameter of about 50 nm to about 900 nm; about 50 nm to about 800 nm; about 50 nm to about 700 nm; about 75 nm to about 600 nm; about 75 nm to about 500 nm; about 100 nm to about 400 nm; or 100 nm to about 300 nm.

In some embodiments, the liposome has a size of or less than about 1 nm, 900 nm, 850 nm, 800 nm; 750 nm; 700 nm; 650 nm; 600 nm; 550 nm; 500 nm, 450 nm, 400 nm, 350 nm, 300 nm, 250 nm, 200 nm, 150 nm, 125 nm, 110 nm, 100 nm, 95 nm, 90 nm, 85 nm, 80 nm, 75 nm, 70 nm, 65 nm, 60 nm, 55 nm, 50 nm, 40 nm, 30 nm or 20 nm.

In the context of various embodiments, the term “liposomal formulation” refers to a formulation of liposomes, wherein liposomes are artificially prepared vesicles made of lipid bilayer. Lipid bilayer may be in a form of a single or one lipid bilayer, or of multiple lipid bilayers. Liposomes may be filled or loaded with drugs. Generally, the liposomes are composed of amphiphilic lipids, such as phospholipids, of natural or synthetic origin. In some embodiments, the membrane properties can be modified by the incorporation of other lipids such as sterols or cholic acid derivatives. In some embodiments, the liposomes comprise one or more of fatty acyls, glycerolipids, phospholipids, glycerophospholipids, sphingolipids, sterol lipids, preno lipids, saccharolipids, and polyketide lipids.

In some embodiments, the liposome comprises a lipid, wherein the lipid includes one or more of phosphatidylcholines, phosphatidylethanolamines, phosphatidic acids, gangliosides, glycolipids, phosphatidylglycerols, and cholesterol. The phosphatidylcholines include, among others, dimyristoylphosphatidylcholine, dipalmitoylphosphatidylcholine, and distearoylphosphatidylcholine. The phosphatidylethanolamines include, among others, dimyristovlphosphatidylethanolamine, dipaimitoylphosphatidylethanolamine, and distearoylphosphatidylethanolamine. The phosphatidic acids include, among others, dimyristoylphosphatidic acid, dipalmitoylphosphatidic acid, distearoylphosphatidic acid, and dicetylphosphoric acid. The gangliosides include, among others, ganglioside GM1, ganglioside GD1a, and ganglioside GT1b. The glycolipids include, among others, galactosylceramide, glucosylceramide, lactosylceramide, phosphatide, and globoside. The phosphatidylglycerols include, among others, dimyristoylphosphatidylglycerol, dipalmitoylphosphatidylglycerol, and distearoylphosphatidylglycerol.

In some embodiments, the phosphatidylcholine is selected from L-α-phosphatidylcholine, egg phosphatidylcholines (EPC), 1,2-dioleoyl-sn-glycero-3-phosphocholines (DOPC), 1,2-dioleoyl-sn-glycero-O-ethyl-3-phosphocholines, 1,2-dilauroyl-sn-glycero-3-phosphocholines (DLPC), 1,2-dimyristoyl-sn-glycero-3-phosphocholines (DMPC), 1,2-dipalmitoyl-sn-glycero-3-phosphocholines (DPPC), 1,2-distearoyl-sn-glycero-3-phosphocholines (DSPC) and mixtures thereof. Phosphatidylcholines may be used alone or in combination with other lipids.

In various embodiments, the phosphatidylcholines can each comprise at least one unsaturated fatty acid moiety. For example, the phosphatidylcholines may each comprise L-α-phosphatidylcholine or 95% egg phosphatidylcholines (EPC). In some embodiments, the sphingolipids may each comprise at least one unsaturated fatty acid moiety. For example, the sphingolipids may each comprise hexadecanoylsphingomyelin or egg sphingomyelin.

In some embodiments, the liposomes include lipids derivatized with a hydrophilic polymer. In some embodiments, the hydrophilic polymer is a biocompatible polymer, particularly for injection into the body, such as intraocular administration. Suitable hydrophilic polymers include, among others, polyvinylpyrrolidone, polyvinylmethylether, polymethyloxazoline, polyethyloxazoline, polyhydroxypropyloxazoline, polyhdroxypropylmethacrylamide, polymethacrylamide, polydimethylacrylamide, polyhydroxypropylmethacrylate, polyhydroxyethylacrylate, hydroxymethylcellulose, hydroxy ethylcellulose, hydroxypropylcellulose, polyethyleneglycol, polyaspartamide, poly-L-lysine, and hydrophilic peptide sequences.

In some embodiments, the lipid in the liposome is derivatized with polyethylene glycol. In some embodiments, the PEG chain has a molecular weight of about 300 to about 5,000 Daltons.

In some embodiments, the liposome is surface modified with poly-L-lysine. In some embodiments, the poly-L-lysine has a molecular weight of about 15,000-30,000 (see, e.g., Sasaki et al., 2013, Eur J Pharm Biopharm. 83(3):364-9; incorporated herein by reference).

Methods of preparing lipids derivatized with hydrophilic polymers are described in, for example U.S. Pat. Nos. 5,395,619; 5,556,948; 6,296,870; WO2014031429; all of which are incorporated herein by reference in their entireties).

In some embodiments, the liposome further comprises cholesterol or derivative thereof. In some embodiments, the cholesterol derivative can be cholestanol, dihydrocholesterol, cholesteryl esters, phytosterol, sitosterol, stigmasterol, campesterol, or mixtures thereof. In liposomes containing cholesterol or derivatives thereof, the amount can vary depending on the need or desired properties. In some embodiments, the content of cholesterol or cholesterol derivative in the liposome is about 0% to about 50% by weight of the liposome, about 0% to about 40% by weight of the liposome, about 0% to about 30% by weight of the liposome, about 0% to about 20% by weight of the liposome, about 0% to about 10% by weight of the liposome, about 10% to about 50% by weight of the liposome, about 10% to about 40% by weight of the liposome, about 10% to about 30% by weight of the liposome, about 10% to about 20% by weight of the liposome, about 20% to about 50% by weight of the liposome, about 20% to about 40% by weight of the liposome, about 20% to about 30% by weight of the liposome, or about 30% to about 40% by weight of the liposome. In some embodiments, the cholesterol content of the liposome is about 10% to about 40% by weight of the liposome.

In some embodiments, the liposome comprises a cationic lipid. In some embodiments, the liposome is a cationic liposome. In some embodiments, the cationic lipid is a linear C₈-C₂₀ alkyl or alkenyl amine. In some embodiments, “C₈-C₂₀ alkyl” refers to a straight chain hydrocarbon group having from 8 to 20 carbon atoms; “C₈-C₂₀ alkenyl” refers to a straight chain hydrocarbon group containing one or more double bonds and having from 8 to 20 carbon atoms. Linear C₈-C₂₀ alkyl or alkenyl amines include, without limitation, dodecyl amine, tallow amine, stearyl amine, cocoamine, octadecyl amine, N-octyloctan-1-amine, 2-nonenylamine, di(2-nonenyl)amine, and mixtures thereof. These linear C₈-C₂₀ alkyl or alkenyl amines may be used alone or in combination. In some embodiments, the linear C₈-C₂₀ alkyl or alkenyl amine is stearyl amine. For example, DPPC may be with and without added stearyl amine. In some embodiments, the cationic lipid is 1,2-di-O-octadecenyl-3-trimethylammoniumpropane (DOTMA), 1,2-dioleyloxy-N,N-dimethylaminopropane (DODMA), dioctadecyldi-muethylammonium (DODA(Br)/DDAB), dioctadecyldimethylammoniumchloride (DODAC), 1,2-dimyristoyloxypropyl-1,3-dimethylhydroxyethylammonium (DMRIE), 2,3-dioleoyloxy-N-[2(spermine carboxamide)ethyl]-N,N-dimethyl-1-propanamium trifluoroacetate (DOSPA) analogues of these molecules having a different composition of the acyl chain moiety. In some embodiments, the cationic liposome includes one or more non-cationic lipid. In some embodiments, the non-cationic lipid is a neutral lipid, for example, cholesterol (Chol) or sphingomyelin (SM).

In some embodiments, the liposome comprises 1,2-dipalmitoyl-sn-glycero-3-phosphocholines (DPPC).

In some embodiments, the liposome comprises egg phosphatidylcholine (EPC) and 1-α-distearoyl phosphatidylcholine.

In some embodiments, the liposomes comprises egg phosphatidylcholine (EPC) or 1,2-dipalmitoyl-sn-glycero-3-phosphocholine), and cholesterol or a derivative thereof.

In some embodiments, the liposome comprises dioleoylphosphatidylcholine (DOPC), dioleoylphosphatidylglycerol (DOPG), and cholesterol or derivative thereof.

In some embodiments, the liposome comprises dipalmitoylphosphatidylcholine (DPPC), palmitoyl-oleoylphosphatidylcholine (POPC), and cholesterol.

In various embodiments, the liposomes can be prepared by various methods available in the art. In some embodiments, the liposomes can be prepared using lipids in aqueous solutions subjected to mechanical agitation, freeze drying, micro-emulsification, sonication, French pressure cells, membrane extrusions, or freeze thawing. In some embodiments, the liposome can be prepared by a solvent dispersion process, including ether injection, ethanol injection, or double emulsion. Methods for preparing liposomes are described in, among others, International patent publication WO 92/10166; Pat. Pub. US20080274172; U.S. Pat. Nos. 4,744,989; 5,395,619; 5,556,948; 5,549,910; 6,296,870; and 8,591,942.

In some embodiments, the loading of the liposomes with a drug compound is done by having the compound present during formation of the liposome, for example, to encapsulate the compound in the liposome. In some embodiments, the compound can be present in the lipid membrane, particularly hydrophobic compounds. In some embodiments, the compound can be incorporated into the liposome subsequent to liposome formation.

In some embodiments, the pharmaceutical composition comprising a compound of the disclosure in a liposome further comprises one or more excipients. In some embodiments, the excipient is one or more of a tonicity agent, viscosity enhancing agent, buffering agent, chelating agent, surfactant, preservative, and antioxidant. In some embodiments, the pharmaceutical liposome composition further comprises a tonicity agent. In some embodiments, the pharmaceutical liposome composition further comprises a buffering agent. In some embodiments, the pharmaceutical liposome composition further comprises a viscosity enhancing agent. In some embodiments, the pharmaceutical liposome composition further comprises a chelating agent. In some embodiments, the pharmaceutical liposome composition further comprises a surfactant. In some embodiments, the pharmaceutical liposome composition further comprises a preservative. In some embodiments, the pharmaceutical liposome composition further comprises an antioxidant. In some embodiments, the excipients are suitable for intraocular, e.g., intravitreal, administration.

In some embodiments, the liposome formulated with the compound further comprises at least a viscosity enhancing agent, as further describe herein. In some embodiments, the viscosity enhancing agent is hyaluronate or hyaluronic acid or a pharmaceutically acceptable salt thereof, as described herein.

In some embodiments, the liposome formulated with the compound is dispersed in a non-ionic surfactant poloxamer (e.g., Pluronic®). In some embodiments, the liposome is dispersed in a poloxamer selected from P124, P188, P237, P338, P407 and mixtures thereof. In some embodiments, the poloxamer is P188, P407, or mixtures thereof (see. e.g., Fattal et al., 2004, Int J Pharm. 277(1-2):25-30; incorporated herein by reference).

### 4.7. Biodegradable Microparticles and Nanoparticles

In another aspect, the compounds of the disclosure are formulated in microparticles or nanoparticles, particularly of bioerodible polymers. Various types of biocompatible bioerodible polymers can be used, including, but not limited to, poly(ester)s, poly(ester amide)s, poly(anhydride)s, poly (carbonate)s, poly (amino acid)s, poly(amide)s, poly(urethane)s, poly(ortho-ester)s, poly(iminocarbonate)s, and poly(phosphazene)s, by themselves or in combination. The polymers can be crosslinked or non-crosslinked. If crosslinked, the polymers can be less than 5% crosslinked, usually less than 1% crosslinked. In some embodiments, microparticle and nanoparticle compositions do not have free aldehyde groups capable of reacting with the compounds described herein.

In some embodiments, the biodegradable polymer comprises hydroxyaliphatic carboxylic acids, either homo- or copolymers, and/or polysaccharides. In some embodiments, the polymers are homo- or copolymers of D-lactic acid, L-lactic acid, racemic lactic acid, glycolic acid, caprolactone, and combinations thereof. In some embodiments, the polymers of the compositions include, among others, poly(lactide) (PLA), poly(lactide-co-glycolide) (PLGA), polyglycolide (PGA), polyhydroxybutyric acid, polycaprolactone (e.g., poly(ε-caprolactone; PCL), polyvalerolactone, poly phosphazene, and polyorthoester. In some embodiments, the PLA can comprise poly D-lactic acid (PDLA) or poly L-lactic acid (PLLA).

In some embodiments, the pharmaceutical compositions comprise block copolymers of polyesters with polyethylene glycol (PEG). PLGA/PEG-block copolymers have been processed as diblock (PLGA-PEG) or triblock molecules with both ABA (PLGA-PEG-PLGA) and BAB (PEG-PLGA-PEG) types. In diblock copolymers, PEG chains orient themselves towards the external aqueous phase in micelles, thus surrounding the encapsulated species. The layer of PEG acts as a barrier and reduces the interactions with other molecules by steric and hydrated repulsion for enhanced shelf stability. In triblock copolymers. ABA and BAB type polymers can act as a thermogel with an A-block covalently coupled with a B-block via ester linkage. The copolymer is usually a free-flowing solution at low temperature and can form a high viscosity gel at body temperature. These temperature-responsive copolymers, PLGA-PEG-PLGA or PEG-PLGA-PEG, are a type of block copolymers composed of hydrophobic PLGA segments and hydrophilic PEG segments. The hydrophobic PLGA segments form associative crosslinks and the hydrophilic PEG segments allow the copolymer molecules to stay in solution. At lower temperatures, hydrogen bonding between hydrophilic PEG segments and water molecules dominates the aqueous solution, resulting in their dissolution in water. As the temperature increases, the hydrogen bonding becomes weaker, while hydrophobic forces among the PLGA segments are strengthened, leading to solution-gel transition.

In some embodiments, the polymers are copolymers of glycolic and lactic acid (e.g., PLGA). In some embodiments, biodegradable polymer matrices include mixtures of hydrophilic and hydrophobic ended PLGA, which are useful in modulating polymer matrix degradation rates. Hydrophobic ended, also referred to as capped or end-capped, PLGA has an ester linkage hydrophobic in nature at the polymer terminus. Typical hydrophobic end groups include, but are not limited to, alkyl esters and aromatic esters. Hydrophilic ended, also referred to as uncapped, PLGA has an end group hydrophilic in nature at the polymer terminus. PLGA with a hydrophilic end group at the polymer terminus typically degrades faster than hydrophobic ended PLGA because it takes up water and undergoes hydrolysis at a faster rate (see, e.g., Tracy et al., 1999, Biomaterials 20:1057-1062, incorporated herein by reference). Examples of suitable hydrophilic end groups that may be incorporated to enhance hydrolysis include, but are not limited to, carboxyl, hydroxyl, and polyethylene glycol.

In some embodiments, the percent of each monomer in poly(lactic-co-glycolic)acid (PLGA) copolymer may be 0-100%, about 15-85%, about 25-75%, or about 35-65%. In some embodiments, the PLGA polymers can have a ratio of lactide to glycolide of about 10:90, 15:85, 20:80, 25:75, 30:70; 35:65, 40:60, 45:55, 50/50, 60:40; 65:35; 70:30 75:25 or 80:20. In some embodiments, the glycolic acid percentage in the polymer is used to adjust the rate of degradation. An increase in glycolic acid percentages generally increases the weight loss of the polymer. For example, PLGA 50:50 (PLA/PGA) exhibits a faster degradation than PLGA 65:35 due to preferential degradation of glycolic acid proportion assigned by higher hydrophilicity. PLGA 65:35 shows faster degradation than PLGA 75:25 and PLGA 75:25 than PLGA 85:15. The glycolic acid content also affects hydrophilicity of the matrix and thus the degradation and drug release rate.

In some embodiments, the microparticles or nanoparticles comprise a poly(ester amide)s (“PEAs”) (see, e.g., Andres-Guerrero et al., 2015, J Controlled Release 211, 10:105-117, incorporated herein by reference). PEAs are synthetic polycondensation products comprised of biocompatible building blocks such as hydrophobic L-amino acids, aliphatic di-carboxylic acids and α,ω diols (see, e.g., patent publication US20170119813A1; Tsitlanadze, et al., 2004, J. Biomater. Sci. Polym. Edit. 15:1-24; U.S. Pat. No. 9,873,764; and Rodriguez-Galin et al., Biodegradable Poly (Ester Amide)s: Synthesis and Applications,” In Biodegradable Polymer: Processing and Degradation, G. Felton ed., Chapter 4, pages 207-272, Nova Science Publishers (2011); all publications incorporated herein by reference in their entireties). In some embodiments, the PEA comprises an α-amino acid (e.g., glycine, 4-amino butyric acid, L-alanine, L-phenylalanine, etc.). In some embodiments, the PEA comprises L-alanine. In some embodiments, the ca-amino acid content is about 100%, about 90%, about 80%, about 70%, about 50%, or about 30%.

In some embodiments, the PEA comprises a copolymer of α-hydroxy acid and α-amino acid. In some embodiments, the PEA comprises poly[(e-caprolactam)-co-(e-caprolactone). In some embodiments, the PEA is a random aliphatic poly(ester amide)s (e.g., based on adipic acid, 6-aminohexanoic acid, and 1,4-butanediol).

In some embodiments, the weight average molecular weight (MW) of the polymers, including copolymers, can be in the range of about 10,000 to about 400,000, or in the range of about 60,000 to about 250.000 Daltons. In some embodiments, the weight average molecular weight (MW) of the polymers is about 8000 to about 14000 Daltons; molecular weight of about 13000 to about 16000 Daltons. Polymers with higher molecular weight, which have longer polymer chains, are used to decrease degradation rates. However this can be opposite for PLLA due to an inversely proportional degree of crystallinity with the molecular weight.

In some embodiments, the pharmaceutical composition is a microsphere, microcapsule, or microparticle. In some embodiments, the microparticles have an average particle size of about 1 to about 250 um, about 1 to about 200 μm; about 1 to about 150 μm; about 1 to about 100 μm; about 1 to about 50 μm; about 1 to about 40 μm; about 1 to about 30 μm; about 1 to about 20 μm; about 1 to about 10 μm; or about 1 to about 5 um.

In some embodiments, the bioerodible pharmaceutical compositions are in the form of a nanoparticle. In some embodiments, the nanoparticles have an average size of about less than about 1 um (sub-micron). In some embodiments, the nanoparticles have an average size of less than about 900 nm, less than about 800 nm, less than about 700 nm, less than about 600 nm, less than about 500 nm, less than about 400 nm, less than about 300 nm, less than about 200 nm, or less than about 100 nm. In some embodiments, the average particle size of the nanoparticle is from about 100 to about 200 nm; about 200 to about 300 nm; about 300 to about 400 nm; or about 400 to about 500 nm. In some embodiments, the average particle size of the nanoparticle is from about 50 to about 900 nm, about 50 to about 800 nm, about 50 to about 700 nm, about 50 to about 600 nm, about 50 to about 500 nm, about 50 to about 400 nm, about 50 to about 300 nm, about 50 to about 200 nm, or about 50 to about 100 nm. In some embodiments, the average particle size is from about 50 to about 500 nm. In other embodiments, the average particle size is from about 50 to about 400 nm. In further embodiments, the average particle size is from about 50 to about 300 nm. In further embodiments, the average particle size is about 50 to about 200 nm. In further embodiments, the average particle size is about 50 to about 100 nm. In further embodiments, the average particle size is about 50 to about 75 nm. In some embodiments, the average particle size is from about 50 to about 60 nm.

In some embodiments of the nanoparticles containing PLGA, the PLGA has an average molecular weight of from about 2,000 to about 10,000 Daltons. In other embodiments, the PLGA has an average molecular weight of from 2,000 to about 7,000 Daltons. In other embodiments, the PLGA has an average molecular weight of from about 2,000 to about 5,000 Daltons. In some embodiments, the PLGA has an average molecular weight of from about 4,000 to about 20,000 Daltons, or from about 4,000 to about 10,000 Daltons, or from about 4,000 to about 5,000 Daltons. In still other embodiments, the PLGA has an average molecular weight of about 2,000, about 4,500, about 5,000, about 7,000, or about 10,000 Daltons.

In some embodiments, the microparticles or nanoparticles can have an associated non-active agent. In some embodiments, the non-active agent is one or more of polyethylene glycols (PEG), fatty acids, amino acids, aliphatic or non-aliphatic molecules, aliphatic thiols, aliphatic amines, and the like. In some embodiments, the non-active agent is a PEG of differing chain lengths. In some embodiments, the non-active agent is a stabilizer, such as polyvinyl alcohol (PVA).

In some embodiments, the pharmaceutical composition is a biocompatible, injectable intraocular drug delivery system comprising (a) a plurality of microparticles with an average diameter of about 8 nm to about 14 um, and (b) an aqueous vehicle for the microspheres, wherein the microspheres comprise: (1) a compound disclosed herein, wherein the compound comprises from about 0.1 wt % to about 10 wt % of the microparticles, and; (2) one or more biodegradable polymers comprising PLA polymers or PLGA polymers with a viscosity of between about 0.4 dL/gm and about 0.8 dL/gm, wherein the PLA polymer or PLGA polymer comprises from about 85 wt % to about 99.5 wt % of the microspheres, and; wherein the drug delivery system can be injected into an intraocular location through a 20 to 26 gauge syringe needle.

In some embodiments, the microparticles and nanoparticles can be prepared by various methods, such as oil/water emulsion, solvent/oil/water emulsion, oil/oil emulsion, and spray drying. Methods are described in, for example, Wischke and Schwendenan, 2008, “Principles of encapsulating hydrophobic compounds in PLA/PLGA microparticles,” Intl J Pharmaceutics 364:298-327; and patent publication US20160143851A1; incorporated herein by reference. In some embodiments, the microparticles are prepared by an oil in water emulsion process by dissolving the polymer (e.g., PLGA) in a water immiscible, volatile organic solvent (such as dichloromethane (DCM), tetrahydrofuran (THF) or ethyl acetate) and then dissolving the compound, sometimes prepared as particles (e.g., 20-30 um) in the prepared solution or alternatively dissolving the compound in a miscible co-solvent and mixing. Co-solvents are generally used for drugs that do not show a high solubility in the primary organic solvent. The resulting organic oil phase is then emulsified in an aqueous solution (continuous phase) containing an appropriate emulsifier, for example polyvinyl alcohol. The emulsifiers included in the aqueous phase can act as stabilizers for the oil-in-water emulsion. The emulsion is then subjected to solvent removal by either evaporation or extraction process to solidify the oil droplets. Generally, volatile solvents can be removed from such emulsions by evaporation to a gas phase or in any case by extraction to the continuous phase. For example, in the former case, the emulsion is maintained at reduced pressure or at atmospheric pressure and the stir rate is reduced while the temperature is increased to enable the volatile solvent to evaporate. In the latter case, the emulsion is transferred to a large quantity of water (with or without surfactant) or other quench medium, into which the solvent associated with the oil droplets is diffused out. Combination of solvent evaporation and extraction is also applicable. The solid microspheres so obtained are then washed and collected by sieving. These are then dried under appropriate conditions such as by vacuum drying or lyophilization.

In some embodiments, the microparticles are prepared by a solvent/oil/water emulsion, which can be used when the compound cannot be dissolved in a carrier solvent or solvent mixture or extensive drug loss to the continuous phase cannot be avoided when employing cosolvent systems. For the solvent/oil/water emulsion, the compound is dispersed in the oil phase of the organic solvent or mixture of solvents and the polymer dissolved into this phase. The s/o/w method generally uses a low drug particle size in order to allow a complete encapsulation of drug crystals.

In some embodiments, the microparticles are prepared by oil/oil emulsion. This method can be used for compounds that are classified as hydrophobic but exhibit some solubility in aqueous media. The compound to be encapsulated and the polymer are dissolved in an organic solvent (e.g., acetonitrile) and then the solution is emulsified into a continuous phase of a solution of an emulsifier (HLB typically <8) in oil, e.g., cottonseed oil or mineral oil. The first oil-phase solvent (e.g., acetonitrile) is extracted in the external oil phase, which can be a non-solvent for both the polymer and the drug. Alternative methods concern the s/o/o technique combining the concepts of s/o/w and o/o methodologies. The removal of the continuous phase can be done by washing the particles with hexane or petroleum ether. The emulsification process can be achieved by the mechanical stirring, high shear mixers and/or static mixers.

In some embodiments, the microspheres or microparticles can be prepared by spraying a solution or suspension of a compound in an organic solution of the polymer, for example a solid-in-oil dispersion or water-in-oil emulsion. Generally, spray drying is defined as the transformation of a feed from a fluid state (solution, or dispersion) into a dried particulate form by spraying the feed into a gaseous drying medium (e.g., hot air). In some embodiments, various spray drying systems can be used, which may be classified according to the nozzle design as rotary atomization, pressure atomization, and two-fluid atomization. In some embodiments, the spray drying process uses coaxial capillary flow technique to produce monodispersed micro/nanoparticles with either simple or core-shell structure.

For preparing nanoparticles, the techniques used in preparing microparticles can be used. Typically, adjustment of processing parameters allows production of nanoparticles. In some embodiments, the process uses a small dispersed phase ratio and rate of stirring. In some embodiments, nanoparticles are prepared by emulsification-solvent evaporation process, particularly for encapsulation of hydrophobic compounds. Double or multiple emulsion can be used for more hydrophilic compounds. Emulsification at high rate of stirring, e.g., high speed homogenizer, can be used to reduce the formed particle size.

In some embodiments, the nanoparticles are prepared by nanoprecipitation process. In some embodiments, polymer and compound are dissolved in an organic solvent (e.g., acetone) and added to an aqueous solution containing surfactant, such as Pluronic, particularly Pluronic F68. The organic solvent is evaporated at appropriate temperatures and reduced pressures leaving behind the polymer encapsulated nanoparticles with drug compound. Salting out is another method in which a water-in-oil emulsion is first formed containing polymer, solvent (usually non-chlorinated solvent, e.g., acetone), salt (e.g., magnesium acetate tetrahydrate) and stabilizer. Water is then added to the solution until the volume is sufficient to diffuse acetone into the water, resulting in nanoparticle formulations

In some embodiments, the pharmaceutical composition of the compound in the form of microparticles or nanoparticles of biodegradable polymer further comprises one or more excipients selected from a tonicity agent, viscosity enhancing agent, buffering agent, chelating agent, surfactant, preservative, and antioxidant.

In some embodiments, the pharmaceutical composition of the compound in the form of microparticles or nanoparticles of a biodegradable polymer further comprises a tonicity agent. In some embodiments, the pharmaceutical composition of the compound in the form of microparticles or nanoparticles of a biodegradable polymer further comprises a buffering agent. In some embodiments, the pharmaceutical composition of the compound in the form of microparticles or nanoparticles of biodegradable polymer further comprises a viscosity enhancing agent. In some embodiments, the pharmaceutical composition of the compound in the form of microparticles or nanoparticles of a biodegradable polymer further comprises a chelating agent. In some embodiments, the pharmaceutical composition of the compound in the form of microparticles or nanoparticles of a biodegradable polymer further comprises a surfactant. In some embodiments, the pharmaceutical composition of the compound in the form of microparticles or nanoparticles of a biodegradable polymer further comprises a preservative. In some embodiments, the pharmaceutical composition of the compound in the form of microparticles or nanoparticles of biodegradable polymer further comprises an antioxidant. In some embodiments, the excipients are suitable for intraocular e.g., intravitreal administration.

### 4.8. Calcium Phosphate Particles

In some embodiments, the pharmaceutical compositions comprise the compound formulated as a calcium phosphate particle, particularly a calcium phosphate nanoparticle (see, e.g., patent publication WO2004050065A1; Chen et al., 2010, Pharm Soc Japan 130(3):419-24; U.S. Pat. No. 8,287,914; patent publication WO2008129562; patent publication EP2041025A2; all publications incorporated herein by reference). The low solubility of the hydroxyapatite (HA) type of calcium phosphate (CaP) in physiological conditions allows it to remain for extended periods after in vivo placement. In some embodiments, the calcium phosphate particles have an average particle size of about 200 nm to about 4000 nm. In some embodiments, the calcium phosphate particles have an average particle size of about 300 nm to about 4000 nm; about 300 nm to about 2000 nm; about 300 nm to about 1000 nm. In some embodiments, calcium phosphate particle compositions do not have free aldehyde groups capable of reacting with the compounds described herein.

In some embodiments, the calcium phosphate particles have an average particle size of less than 1000 nm (sub-micron size). In some embodiments, the calcium phosphate particles have an average particle size of about 200 to less than 1000 nm; about 200 to about 900 nm; about 200 to about 800 nm; about 200 to about 600 mm, or about 200 to about 400 nm. In some embodiments, the core particles of the nanoparticles have a morphology that is generally and substantially spherical in shape.

In some embodiments, the calcium phosphate nanoparticles can have varying stoichiometric amounts of Ca²⁺ and PO₄³⁻ ions. In some embodiments, the substitution of some ions of the crystallographic structure by ions such as P, Na⁺, K⁺, Mg²⁺ and CO₃²⁺ can provide different properties to the substituted calcium phosphates. In some embodiments, the calcium phosphates is hydroxyapatite (HA). Generally, the reabsorbing rate is proportional to the calcium phosphate solubility, which is also affected by the pH. Generally, CaP's can be ordered by their decreasing solubility as follows: dicalcium phosphate dihydrate (DCPD)>dicalcium phosphate anhydrous (DCPA)>amorphous calcium phosphate (ACP)>tetracalcium phosphate (TTCP)>α-tricalcium phosphate (α-TCP)>octacalcium phosphate (OCP)>tricalcium phosphate (β-TCP)>hydroxyapatite. In some embodiments, the calcium phosphate particle comprises dicalcium phosphate dihydrate (DCPD), dicalcium phosphate anhydrous, amorphous calcium phosphate (ACP), tetracalcium phosphate (TTCP), α-tricalcium phosphate (α-TCP), octacalciunm phosphate (OCP), tricalcium phosphate (β-TCP), or hydroxyapatite. In some embodiments, the calcium phosphate particles comprise calcium phosphate, calcium hydroxyapatite, alpha tricalcium phosphate, beta tricalcium phosphate, calcium pyrophosphate, tetracalcium phosphate, or octacalcium phosphate. In some embodiments, the calcium phosphate particle comprises hydroxyapatite. In some embodiments, the calcium phosphate is β-tricalcium phosphate (β-TCP) that is a bioresorbable.

In some embodiments, the calcium phosphate particles can be prepared as a suspension in aqueous medium by reacting a soluble calcium salt with a soluble phosphate salt, and more particularly, by reacting calcium chloride with sodium phosphate under aseptic conditions. For example, an aqueous solution of calcium chloride having a concentration between about 5 mM and about 300 mM is combined by mixing with an aqueous solution of a suitable distilled water-based solution of sodium citrate, having a concentration between about 5 mM and about 300 mM. The presence of sodium citrate contributes to the formation of an electrostatic layer around the core particle, which helps to stabilize the attractive and repulsive forces between the core particles, resulting in physically stable calcium phosphate core particles. An aqueous solution of dibasic sodium phosphate having a concentration between about 5 mM and about 300 mM is then mixed with the calcium chloride/sodium citrate solution. The solution generally become turbid, indicating the formation of calcium phosphate core particles. In some embodiments, mixing is generally continued for at least about 48 hours, or until a suitable core particle size has been obtained, as determined by sampling the suspension and measuring the core particle size using known methods. The core particles may be optionally stored and allowed to equilibrate for a sufficient period of time, e.g., about seven days, at room temperature to achieve stability in size and pH prior to further use.

In some embodiments, the calcium phosphate particle has a calcium/phosphate molar ratio selected from a range of about 1 to 400, about 2 to 400, about 5 to 400, about 10 to 400, about 15 to 400, about 20 to 400, about 25 to 400, about 50 to 400, about 75 to 400, about 100 to 400, about 150 to 400, or about 200 to 400. In some embodiments, the calcium to phosphate molar ratio is about 2 to 350, about 5 to 300, about 10 to 250, about 15 to 200, about 20 to 150, about 25 to 100, or about 50 to 75. In some embodiments, the calcium phosphate particle has a calcium/phosphate molar ratio (Ca/P) of 1, 2, 5, 10, 15, 20, 25, 50, 75, 100, 150, 200, 250, 300, 350 or 400.

In some embodiments, the particles can be at least partially coated or impregnated or both with a pharmacologically active agent, wherein the pharmacologically active agent is disposed on the surface of the core particle and optionally held in place by a surface modifying agent sufficient to bind the agent to the core particle. In some embodiments, the surface modifying agents suitable for use in the particles include modified carbohydrates, carbohydrate derivatives, and other macromolecules with carbohydrate-like components characterized by the abundance of —OH side groups; polyethylene glycol (PEG); and modified PEG, such as PEG-inositol 1,3,4,5,6-pentakisphosphate (PEG-IP5). Surface modifying agents are described in, for example, U.S. Pat. Nos. 5,460,830; 5,462,751; 5,460,831; 5,219,577; Huang et al., 2017, ACS Appl. Mater. Interfaces 9 (12):10435-10445; the entire contents of each of which are incorporated herein by reference.

In some embodiments, the calcium phosphate particle is coated with a lipid (see, e.g., Huang et al., 2018, J Drug Targeting 26(5-6)). In some embodiments, the lipid is one or more of phosphatidylcholines, phosphatidylethanolamines, phosphatidic acids, gangliosides, glycolipids, phosphatidylglycerols, and cholesterol. In some embodiments, the phosphatidylcholine is L-α-phosphatidylcholine, egg phosphatidylcholines (EPC), 1,2-dioleoyl-sn-glycero-3-phosphocholines (DOPC), 1,2-dioleoyl-sn-glycero-O-ethyl-3-phosphocholines, 1,2-dilauroyl-sn-glycero-3-phosphocholines (DLPC), 1,2-dimyristoyl-sn-glycero-3-phosphocholines (DMPC), 1,2-dipalmitoyl-sn-glycero-3-phosphocholines (DPPC), 1,2-distearoyl-sn-glycero-3-phosphocholines (DSPC) and mixtures thereof.

In some embodiments, the lipid coating on the calcium phosphate particle is 1,2 dioleoyl-3-trimethylammonium propane (DOTAP) and cholesterol. In some embodiments, the lipid coating on the calcium phosphate particle is soy lecithin and cholesterol.

Coating of the core particles with a pharmacologically active agent can be carried out by suspending the core particles in a solution containing a dispersed surface modifying agent, for example a solution of water containing from about 0.1 to about 30 wt % of the surface modifying agent. The cores are maintained in the surface modifying agent solution for a suitable period of time, generally about one hour, and may be agitated, e.g., by rocking or sonication. The coated core particles can be separated from the suspension, including from any unbound surface modifying agent, by centrifugation. The coated core particles can then be resuspended in a solution containing the pharmacologically active agent to be adhered to the at least partially coated core particle. Optionally, a second layer of surface modifying agent may also be applied to the pharmacologically active agent adhered to the particle.

In some embodiments, the pharmaceutical composition comprising a compound of the disclosure formulated as calcium phosphate nanoparticles further comprises one or more suitable excipients. In some embodiments, the excipient is one or more of a tonicity agent, buffering agent, viscosity enhancing agent, chelating agent, surfactant, preservative, and antioxidant.

In some embodiments, the calcium phosphate nanoparticle compositions further comprise a tonicity agent. In some embodiments, the calcium phosphate nanoparticle compositions further comprise a buffering agent. In some embodiments, the pharmaceutical calcium phosphate nanoparticle compositions further comprise a viscosity enhancing agent. In some embodiments, the pharmaceutical calcium phosphate nanoparticle compositions further comprise a chelating agent. In some embodiments, the pharmaceutical calcium phosphate nanoparticle compositions further comprise a surfactant. In some embodiments, the pharmaceutical calcium phosphate nanoparticle compositions further comprise a preservative. In some embodiments, the pharmaceutical calcium phosphate particle compositions further comprise an antioxidant. In some embodiments, the excipients are suitable for intraocular, e.g., intravitreal, administration.

### 4.9. Complexing Agents

In some embodiments, the compounds of the disclosure are prepared as pharmaceutical composition comprising a complexing agent. A complexing agent is capable of complexing with one or more endogenous components of the tissue or tissue fluids, such as the vitreous, for forming a mass of enhanced viscosity. The complexing can occur through ionic interaction between the complexing agent and one or more components of the tissue, although other interaction (e.g., chemical reaction) may alternatively or additionally form the complex. In some embodiments, the complexing agent is cationically (i.e., positively) charged such that it can form an ionic complex with endogenous hyaluronic acid, collagen or both in the tissue or tissue fluid to form the mass of enhanced viscosity. In some embodiments, the complexing agent can be a positively charged polymer. In some embodiments, the complex formed between the complexing agent and the endogenous tissue component (e.g., hyaluronic acid) and the mass of enhanced viscosity formed thereby be bioerodible within the tissue to aid in the gradual breakdown of the mass and/or complex after formation thereof. In some embodiments, pharmaceutical compositions of a complexing agent do not have free aldehyde groups capable of reacting with the compounds described herein.

In some embodiments, the complexing agent is, by way of example and not limitation, polyamino acid, galactomannan (e.g., cationic-derivatized), cellulosic polymers, amine compounds, quaternary ammonium compounds or any combinations thereof. In some embodiments, the complexing agents can be provided in a polymeric and/or positively charged form. The complexing agent can be present in the composition in an amount that is at least 0.01 w/v %, at least 0.1 w/v %, or at least 0.5 w/v %. In some embodiments, the concentration of complexing agent will also typically be no greater than about 10 is w/v %, more typically no greater than about 3 w/v % and even possibly no greater than 1.0 w/v %.

In some embodiments, the complexing agent is a poly-amino acid formed of multiple repeat units of an amino acid. Exemplary poly-amino acid complexing agents include, without limitation, polylysine, polyarginine, polyhistidine, or the like. In some embodiments, the polyamino acid, when used, is typically present in the composition at a concentration of at least 0.05% w/v, at least 0.2% w/v, at least 0.7% w/v, or at least 1% w/v. In some embodiments, the poly-amino acid is at a concentration that is generally less than 10% w/v, at less than 5.0% w/v, or less than 1.4% w/v. Exemplary polylysines include poly-L-lysine, poly-D-lysine, racemic poly-DL-lysine, derivatives thereof and combinations thereof. In some embodiments, any of alpha polylysines, epsilon polylysines, poly-L-lysines, poly-D-lysines, any derivatives thereof, any combinations thereof or the like may be used for the pharmaceutical compositions. In some embodiments, the complexing agent is poly-c-L-lysine. In some embodiments, the lysine of the composition may be entirely or substantially entirely poly-c-L-lysine. The term substantially entirely, as it refers to poly-c-L-lysine means at least 70% by weight and more preferably at least 90% by weight of the lysine of the composition is poly-c-L-lysine.

In some embodiments, the polylysine in the composition has an average molecular weight that is at least 50,000 Daltons, more typically at least 150,000 Daltons or at least 300,000 Daltons. In some embodiments, the average molecular weight of the polylysine is from about 50,000 to about 500.000 Daltons; about 50,000 to about 400,000 Daltons; or about 50,000 to about 300,000 Daltons

In some embodiments, the complexing agent is a positively charged amine compound, particularly positively charged amine polymers. The amine polymers can be primary, secondary, tertiary amines or a combination thereof. Such amine compounds or amine polymers can include or be derived from aromatic or zo heterocyclic base groups such as aniline, pyridine or others. Nucleosides and polymers derived therefrom are one particularly preferred class of amine compounds suitable as complexing agents for the composition of the present invention. Poly saccharides containing amine groups are also preferred for the composition of the present invention. Examples of amine containing polysaccharides include chitosan and water-soluble derivatives of chitosan.

In some embodiments, the complexing agent is a derivative of natural polymers, which has been modified to be positively charged and/or soluble in water, Cellulosic polymers are preferred within this class. In some embodiments, the complexing agent comprises a positively charged cellulosic polymer. In some embodiments, the cellulosic polymer comprises a copolymer of polyethoxylated cellulose or dimethyldiallyl ammonium chloride. In some embodiments, the polymers comprise CELQUAT SC-230M® and CELQUAT SC-240C® (Akzo-Nobel). Advantageously, these polymers can be modified to include varying amounts of nitrogen (i.e., nitrogen substitutions) and, through the use of greater or lesser substitutions, the degree of complexing can respectively be raised or lowered. When included, the positively charged natural (e.g., cellulosic) polymers are typically present in the composition at a concentration that is at least 0.01 w/v %, more typically at least 0.05 w/v % and even more typically at least 0.2 w/v % and a concentration that is typically less than 4.0 w/v %, more typically less than 1.0 w/v % and even more typically less than 0.4 w/v %.

In some embodiments, the complexing agent is a quaternary ammonium compound. A variety of quaternary copolymers of varying quaternization can be synthesized based on homo or copolymers of amino acrylates with methyl, ethyl or propyl side chains. These monomers could also be copolymerized with other nonionic monomers including quaternary acrylic homopolymers such as homopolymers of 2-methacryloxyethyl trimethylammonium chloride and 2-methacryloxyethyl methyl diethyl ammonium bromide and copolymers of quaternary acrylate monomers with water soluble monomers. When included, the quaternary ammonium compounds can be present in the composition at a concentration of at least about 0.01 w/v %, more typically at least 0.05 w/v %, and even more typically at least 0.2 w/v %, and a concentration that is typically less than 4.0 w/v %, more typically less than 1.0 w/v % and even more typically less than 0.4 w/v %.

A useful polymer complexing agent is a polymeric quaternary ammonium salt of hydroxyethylcellulose and a trimethyl ammonium chloride substituted epoxide. This complexing agent is both a quaternary ammonium compound and a cellulosic polymer and has the CTFA designation polyquaternium-10. A suitable polymer is sold under the tradename UCARE JR-30M, which is commercially available from Rhodia or CELQUAT L-200 and H-100 (Akzo Nobel). In some embodiments, a suitable quaternary ammonium/cellulosic agent is an alkyl modified quaternary ammonium salt of hydroxyethyl cellulose and a trimethyl ammonium chloride substituted epoxide having the CTFA designation polyquaternium-24. An example of such polymer is sold under the tradename QUATRISOFT LM-200 and is commercially available from Amerchol Corp., Edison, N.J. Other useful polymer complexing agents, which are both quaternary ammonium compounds and cellulosic polymers, include various quaternary ammonium salts of hydroxy ethyl cellulose sold under the tradename SOFTCAT and commercially available from The Dow Chemical Company, Midland, Mich.

In some embodiments, the complexing agent is a galactomannan polymer, for example cationic-derivatized galactomannan polymer, which can also typically be considered a cellulosic polymer. A useful polymer is positively charged guar. Guar (e.g., guar gum) or other galactomannan polymer substituted with positively charged chemical moieties can be used. In some embodiments, the galactomannan polymer can have a cationic degree of substitution (DS) with a lower limit of 0.01% and an upper limit of 3.0%, preferably a lower limit of 0.1% or 0.3% and an upper limit of 2.5%. The galactomannan, particularly in the case of guar gum, typically has a number weight average molecular weight (MW) with a lower limit of 50,000 and an upper limit of about 1,000,000, more preferably a lower limit of 100, 000 or 300,000 and an upper limit of about 700,000. An exemplary galactomannan is a positively charged guar gum such as O-[2-hydroxy-3-(trimethylammonium) propyl] chloride guar, which is commercially available under the tradename C261N from Cosmedia. In some embodiments, the galactomannan polymer (e.g., guar gum) compounds exhibit low toxicity. In some embodiments, the galactomannan polymer is typically present in the composition at a concentration that is at least 0.04 w/v %, more typically at least 0.20 w/v % and even more typically at least 0.5 w/v % and a concentration that is typically less than 7.0 w/v %, more typically less than 3.0 w/v % and even more typically less than 1.2 w/v %.

In some embodiments, the pharmaceutical composition comprising the compound of interest and the complexing agent is formulated as a solution or a suspension. In some embodiments, the pharmaceutical composition is aqueous and comprises at least 50% and more typically at least 95% water.

In some embodiments, for intraocular or intravitreal injection, the composition prepared with the complexing agent consist essentially of or substantially of only complexing agent, therapeutic compound and water. As used herein, substantially only complexing agent, therapeutic agent and water means that the composition includes less than 5.0 w/v %, more typically less than 4.0 w/v and even more preferably less than 2.0 w/v % of any ingredients other than complexing agent, therapeutic agent and water.

In some embodiments, the pharmaceutical composition of the compound and a complexing agent further comprises one or more excipients selected from a tonicity agent, viscosity enhancing agent, buffering agent, chelating agent, surfactant, preservative, and antioxidant.

In some embodiments, a suspending agent may be employed. In some embodiments, the suspending agent is, without limitation, a polysaccharide, e.g., xanthan gum, carboxymethylcellulose, chondroitin sulfate, or carboxyvinyl polymer.

### 4.10. Cyclodextrin Compositions

In some embodiments, the compounds of the disclosure are formulated as a pharmaceutical composition with a cyclodextrin. Formulations of an aldehyde trapping compound and cyclodextrin are disclosed in U.S. Pat. No. 9,814,701, incorporated herein by reference. In some embodiments, the pharmaceutical composition comprises a compound of the present disclosure, a cyclodextrin, and one or more excipients. In some embodiments, the excipients are suitable for intraocular, e.g. intravitreal administration.

In some embodiments, the cyclodextrin for use in the methods and compositions is selected from α-cyclodextrin, β-cyclodextrin, γ-cyclodextrin, derivatives thereof, and combinations thereof. In particular, the cyclodextrin for use in the methods is selected from β-cyclodextrin, γ-cyclodextrin, derivatives thereof, and combinations thereof.

In some embodiments, the cyclodextrin or derivative thereof is selected from carboxyalkyl cyclodextrin, hydroxy alkyl cyclodextrin, sulfoalkylether cyclodextrin, and alkyl cyclodextrin. In various embodiments, the alkyl group in the cyclodextrin is methyl, ethyl, propyl, butyl, pentyl, or hexyl.

In some embodiments, the cyclodextrin is α-cyclodextrin or a derivative thereof. In some embodiments, the α-cyclodextrin or a derivative thereof is selected from carboxyalkyl-α-cyclodextrin, hydroxyalkyl-α-cyclodextrin, sulfoalkylether-α-cyclodextrin, alkyl-α-cyclodextrin, and combinations thereof. In some embodiments, the alkyl group in the α-cyclodextrin derivative is methyl, ethyl, propyl, butyl, pentyl or hexyl.

In some embodiments, the cyclodextrin is β-cyclodextrin or a derivative thereof. In some embodiments, the β-cyclodextrin or derivative thereof is selected from carboxyalkyl-β-cyclodextrin, hydroxyalkyl-β-cyclodextrin, sulfoalkylether-β-cyclodextrin, alkyl-β-cyclodextrin, and combinations thereof. In some embodiments, the alkyl group in the β-cyclodextrin derivative is methyl, ethyl, propyl, butyl, pentyl or hexyl.

In some embodiments, the β-cyclodextrin or a derivative thereof is hydroxyalkyl-β-cyclodextrin or sulfoalkylether-β-cyclodextrin. In some embodiments, the hydroxyalkyl-β-cyclodextrin is hydroxypropyl-β-cyclodextrin. In some embodiments, the sulfoalkylether-β-cyclodextrin is sulfobutylether-β-cyclodextrin. In some embodiments, β-cyclodextrin or a derivative thereof is alkyl-β-cyclodextrin, in particular methyl-β-cyclodextrin. In some embodiments using methyl-β-cyclodextrin, the β-cyclodextrin is randomly methylated β-cyclodextrin.

In some embodiments, the cyclodextrin is γ-cyclodextrin or a derivative thereof. In some embodiments, the γ-cyclodextrin or derivative thereof is selected from carboxyalkyl-γ-cyclodextrin, hydroxyalkyl-γ-cyclodextrin, sulfoalkylether-γ-cyclodextrin, and alkyl-γ-cyclodextrin. In some embodiments, the alkyl group in the γ-cyclodextrin derivative is methyl, ethyl, propyl, butyl, pentyl, or hexyl. In some embodiments, the γ-cyclodextrin or derivative thereof is hydroxyalkyl-γ-cyclodextrin or sulfoalkylether-γ-cyclodextrin. In some embodiments, the hydroxyalkyl-γ-cyclodextrin is hydroxypropyl-γ-cyclodextrin, such as 2-hydroxypropyl-γ-cyclodextrin. In some embodiments, the γ-cyclodextrin or derivative thereof is S-2-carboxyalkyl-thio-γ-cyclodextrin, such as S-2-carboxyethyl-thio-γ-cyclodextrin.

In some embodiments, various salts of the cyclodextrin or salts of the cyclodextrin derivative can be used in the compositions and methods herein. In some embodiments, the salts are pharmaceutically acceptable salt(s), which refers to those salts of compounds, i.e., cyclodextrin, that are safe and effective for use in mammals and that possess the desired biological activity. Pharmaceutically acceptable salts include salts of acidic or basic groups present in the cyclodextrins. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, and p-toluenesulfonate salts. Suitable base salts include, but are not limited to, aluminum, calcium, lithium, magnesium, potassium, sodium, zinc, and diethanolamine salts. In some embodiments, the cyclodextrin is in the form of a sodium or potassium salt. Guidance on suitable pharmaceutically acceptable salts and their application to drug formulations can be found in various references, such as Remington's Pharmaceutical Sciences, 17th Ed., Mack Publishing Company, Easton, Pa., 1985, and Berge, et al., 1977, “Pharmaceutical Salts,” J Pharm Sci. 66:1-19, both of which are incorporated herein by reference.

In some embodiments, the cyclodextrin is a mixture of cyclodextrins. Such mixtures can be a combination of: α-cyclodextrin and β-cyclodextrin, including combinations of α-cyclodextrin and β-cyclodextrin derivatives; α-cyclodextrin and γ-cyclodextrin, including combinations of α-cyclodextrin and γ-cyclodextrin derivatives; β-cyclodextrin and γ-cyclodextrin, including combinations of β-cyclodextrin and γ-cyclodextrin derivatives; or combinations of α-cyclodextrin, β-cyclodextrin, and γ-cyclodextrin, including combinations of α-cyclodextrin, β-cyclodextrin, and γ-cyclodextrin derivatives.

In some embodiments, the cyclodextrin (e.g., α-, β-, or γ-cyclodextrine), such as in a composition as described herein is present at about 0.1% to about 30% w/v, about 0.1% to about 25% w/v, about 0.1% to about 20% w/v, about 0.2% to about 15% w/v, about 0.5% to about 10% w/v, about 0.5% to about 7.5% w/v, or about 1% to about 5% w/v. For example, an exemplary cyclodextrin is sulfobutylether-β-cyclodextrin, which can be present at about 0.1% to about 30% w/v, about 0.1% to about 25% w/v, about 0.1% to about 20% w/v, about 0.2% to about 15% w/v, about 0.5% to about 10% w/v, about 0.5% to about 7.5% w/v, or about 1% to about 5% w/v. In some embodiments, an exemplary cyclodextrin is hydroxypropyl-β-cyclodextrin, which can be present at about 0.1% to about 30% w/v, about 0.1% to about 25% w/v, about 0.1% to about 20% w/v, about 0.2% to about 15% w/v, about 0.5% to about 10% w/v, about 0.5% to about 7.5% w/v, or about 1% to about 5% w/v. In some embodiments, an exemplary cyclodextrin is hydroxypropyl-γ-cyclodextrin, which can be present at about 0.1% to about 30% w/v, about 0.1% to about 25% w/v, about 0.1% to about 20% w/v, about 0.2% to about 15% w/v, about 0.5% to about 10% w/v, about 0.5% to about 7.5% w/v, or about 1% to about 5% w/v.

In embodiments where mixtures of cyclodextrins are used, for example mixtures of sulfobutylether-β-cyclodextrin and hydroxypropyl-β-cyclodextrin, the total amount of cyclodextrin can be present at about 0.1% to about 30% w/v, about 0.1% to about 25% w/v, about 0.1% to about 20% w/v, about 0.2% to about 15% w/v, about 0.5% to about 10% w/v, about 0.5% to about 7.5% w/v, or about 1% to about 5% w/v.

In some embodiments, the cyclodextrin, such as in a composition thereof, in particular an ophthalmic solution, for use in the methods herein is present at about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 18% w/v, about 20% w/v, about 25% w/v, or about 30% w/v. For example, a β-cyclodextrin, e.g., sulfobutylether-β-cyclodextrin, can be present at about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/% v, about 15% w/v, about 16% w/v, about 18% w/v, about 20% w/v, about 25% w/v, or about 30% w/v. In some embodiments, a β-cyclodextrin, e.g., hydroxypropyl-β-cyclodextrin, can be present at about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 18% w/v, about 20% w/v, about 25% w/v, or about 30% w/v. In some embodiments, a γ-cyclodextrin, e.g., hydroxypropyl-7-cyclodextrin, can be present at about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 18% w/v, about 20% w/v, about 25% w/v, or about 30% w/v.

In some embodiments where mixtures of cyclodextrins are used, for example mixtures of sulfobutylether-β-cyclodextrin and hydroxypropyl-β-cyclodextrin, the total amount of cyclodextrin can be present at about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 18% w/v, about 20% w/v, about 25% w/v, or about 30% w/v.

It is to be understood that while the cyclodextrin levels (e.g., concentration) for administration are given for exemplary cyclodextrins sulfobutylether-β-cyclodextrin, hydroxypropyl-β-cyclodextrin, and hydroxypropyl-γ-cyclodextrin, equivalent concentrations of the other specific cyclodextrins can be readily determined by the person of skill in the art.

In various embodiments containing the compounds formulated with a cyclodextrin, the compositions comprise one or more excipients. In some embodiments, the excipients are one or more of a tonicity agent, buffering agent, viscosity enhancing agent, chelating agent, surfactant, preservative, and antioxidant. In some embodiments, the cyclodextrin composition includes a tonicity agent. In some embodiments, the cyclodextrin composition includes a buffering agent. In some embodiments, the cyclodextrin composition includes a viscosity enhancing agent. In some embodiments, the cyclodextrin composition includes a chelating agent. In some embodiments, the cyclodextrin composition includes a surfactant. In some embodiments, the cyclodextrin composition includes an anti-oxidant. In some embodiments, the cyclodextrin composition includes a preservative.

In some embodiments, the composition of the compound and cyclodextrin containing one or more excipients are suitable for intraocular, e.g., intravitreal administration. In some embodiments, the composition of the compound and cyclodextrin containing one or more excipients are suitable for subcutaneous, intradermal, or intramuscular administration. In some embodiments, the composition of the compound and cyclodextrin containing one or more excipients are formulated to be suitable for intravenous administration.

In some embodiments, the pharmaceutical composition of the compound and cyclodextrin does not include carboxymethylcellulose. In some embodiments, the pharmaceutical composition of the compound and cyclodextrin does not include hydroxypropylcellulose. In some embodiments, where hydroxypropylmethylcellulose is present, the concentration is greater than 1% w/v.

### 4.11. Injectable Hydrogels

In some embodiments, the pharmaceutical composition comprises a compound of the disclosure and a hydrogel, in particular an injectable hydrogel. Generally, injectable hydrogels can undergo a “sol-gel” transition, where the transition is triggered by a change in environmental condition. In some embodiments, the condition for “sol-gel” transition include properties such as pH, temperature, ionic environment, glucose, or combinations thereof. Injectable hydrogels can be injected into a tissue, organ (e.g., eye) or a part of the body in the form of liquid and once administered undergo in situ gelation (see, e.g., US20160331738; WO2006122183A2; incorporate herein by reference). The compound of interest is incorporated and suspended in the hydrogel in the “sol” state. In some embodiments, hydrogel compositions do not have free aldehyde groups capable of reacting with the compounds described herein.

In some embodiments, the hydrogel is at concentrations in which the composition is liquid at conditions prior to administration, and forms a gel upon administration into the tissue, organ or body part. For example, at concentrations of 20% w/v and higher, aqueous solutions of Poloxamer F407, described below, remain as a liquid at low temperatures (<15° C.) and transitions to a viscous semisolid gel upon intraocular administration.

In some embodiments, the injectable hydrogels are comprised of polyethylene oxide; polypropylene oxide; polyoxyethylene-polyoxypropylene block copolymers (e.g., Poloxamer 407 and Poloxamer 188); acrylic polymers such as, but not limited to, polyacrylic acid (PAA) (e.g., carbomers, Carbopol® 974P, etc.); polyvinylpyrrolidones; polyethylene glycols (PEGs) (e.g., from Nektar); gelatin, polyvinyl alcohols (PVA); polyhydroxyethyl methacrylate (poly-HEMA or PHEMA); cellulose; alginate; chitin or combinations thereof.

In some embodiments, the hydrogel is a biodegradable hydrogel. A biodegradable hydrogel can incorporate a biodegradable linkage in the hydrogel and/or precursor may be water-degradable or enzymatically degradable. Exemplary water-degradable biodegradable linkages include polymers, copolymers and oligomers of glycolide, dl-lactide, l-lactide, dioxanone, esters, carbonates, and trimethylene carbonate. Exemplary enzymatically biodegradable linkages include peptidic linkages cleavable by metalloproteinases and collagenases. Examples of biodegradable linkages include polymers and copolymers of poly(hydroxy acid)s, poly(orthocarbonate)s, poly(anhydride)s, poly(lactone)s, poly (amino acid)s, poly(carbonate)s, and poly(phosphonate)s. In some embodiments, the gel degrades in the physiological fluid in without causing inflammation by degrading into components that are biocompatible and not acidic. In some embodiments, the hydrogel adheres to the tissue.

In some embodiments, the hydrogel is a thermoresponsive hydrogel. In certain embodiments, the thermoresponsive hydrogel has a lower critical solution temperature (LCST) below body temperature. In some embodiments for therapeutic use, the thermoresponsive hydrogel remains fluid below physiological temperature (e.g., 37° C. for humans) or at or below room temperature (e.g., 25° C.) and forms into a hydrogel at the physiological temperature and is biocompatible. In some embodiments, the thermoresponsive hydrogel can be a liquid at a temperature below 34° C. which reversibly solidifies into a gelled composition at a temperature above 34° C. In some embodiments, the hydrogel composition is a solution at about 20° C. to 27° C. and is a gel at about 34° C. to 37° C.

Thermoresponsive hydrogel can be selected from naturally derived and synthetic polymers exhibiting this behavior. Natural polymers include elastin-like peptides and polysaccharides derivatives, while synthetic polymers include those comprised of poly(n-isopropyl acrylamide) (PNIPAAm), poly(N,N-dimethylacrylamide-co-N-phenylacrylamide), poly(glycidyl methacrylate-co-N-isopropylacrylamide), poly(ethylene oxide)-β-poly(propylene oxide)-β-poly(ethylene oxide), poly(ethylene glycol)-polyester copolymer, and amphiphilic block copolymers. In some embodiments, the amphiphilic block copolymer comprises a hydrophilic component selected from polyethylene oxide (PEO), polyvinyl alcohol (PVA), polyglycolic acid (PGA), poly(N-isopropylacrylamide), poly(acrylic acid) (PAA), polyvinyl pyrrolidone (PVP) or mixtures thereof, and a hydrophobic component selected from polypropylene oxide (PPO), poly (lactic acid) (PLA), poly(lactic acid co glycolic acid) (PLGA), poly(β-benzoyl L-aspartate) (PBLA), poly(γ-benzyl-L-glutamate) (PBLG), poly(aspartic acid), poly(L-lysine), poly(spermine), poly(caprolactone), or mixtures thereof. Exemplary amphiphilic block copolymers include (PEO)(PPO)(PEO) block copolymers (PEO/PPO), and poly(lactic acid co glycolic acid) block copolymers (PLGA), such as (PEO)(PLGA)(PEO) block copolymers.

In some embodiments, the hydrogel is comprised of polyacrylamide (e.g., poly-N-isopropylacrylamide), polyethylene oxide/polypropylene oxide, or combinations of the two (e.g., Pluronic® or Tectronic®), butyl methacrylate, polyethylene glycol diacrylate, polyethylene glycol of varying molecular weights, polyacrylic acid, poly-methacrylic acid, poly-lactic acid, poly(tetramethylene ether glycol), poly(N,N′-diethylaminoethyl methacrylate), methyl methacrylate, and N,N′-dimethylaminoethylmethacrylate.

In some embodiments, the hydrogel is selected from xyloglucan, gelatin, agarose, amylase, amylopectin, cellulose derivatives and gellan, exhibiting thermoreversible gelation behaviors. For example, a chitosan-based, injectable thermogels has been engineered through grafting an appropriate amount of PEG onto the chitosan backbone and used for prolonged drug release in vitro (see. e.g., U.S. Pat. Nos. 6,730,735; 8,663,686; incorporated herein by reference).

In some embodiments, the hydrogel is comprised of poly(ethylene oxide)-β-poly(propylene oxide)-β-poly(ethylene oxide), also known as poloxamer, Phuronics®, or Tetronics®. In some embodiments, the poloxamer is P124, P188, P237, P338, P407, or mixtures thereof (e.g., Pluronic®, e.g., L 44 NF, F 68 NF, F 87 NF, F 108 NF, and F 127 NF). In some embodiments, the hydrogel is comprised of P188, or P407, or mixtures thereof. In some embodiments, the hydrogel is a mixture of P188 and P407. In some embodiments, the poloxamer hydrogel can include an additional polymer, such as polyethylene glycol, PAA, methylcellulose, hydroxypropyl methylcellulose, carboxymethyl cellulose to achieve a phase transition temperature higher than room temperature (25° C.) and gelling at tissue, organ or body temperature (e.g., pre-corneal temperature 35° C.).

In some embodiments, the hydrogel is a pH sensitive hydrogel. In some embodiments, the hydrogel is selected from polymethyl methacrylate (PMMA), polyacrylamide (PAAm), polyacrylic acid (PAA), poly-dimethylaminoethylmethacrylate (PDEAEMA) and polyethylene glycol. These polymers are generally hydrophobic but swells in water depending upon the pH prevalent in the external environment. In some embodiments, the pH-sensitive hydrogel can be a copolymer of PMMA and polyhydroxyethyl methyl acrylate (PHEMA), which are anionic copolymers, swell high in neutral or high pH but do not swell in acidic medium.

In some embodiments, the hydrogel comprises a carbomer (e.g., Carbopol®), which is a cross-linked acrylic acid polymer (PAA) and shows pH induced phase transition as the pH is raised above its pKa of about 5.5. Carbomers are available as carbomer homopolymers, which are polymers of acrylic acid crosslinked with allyl sucrose or allyl pentaerythritol; carbomer copolymers, which are polymers of acrylic acid and C₁₀-C₃₀ alkyl acrylate crosslinked with allyl pentaerythritol; and carbomer interpolymers, which are carbomer homopolymer or copolymer that contains a block copolymer of polyethylene glycol and a long chain alkyl acid ester. In some embodiments, the carbomer is selected from Carbopol 71G NF, 971P NF, 974P NF, 980 NF, 981 NF, 5984 EP, and ETD 2020 NF, Ultrez 10 NF. In some embodiments, the hydrogel comprises a carbomer and a suitable viscosity enhancing agent, e.g. hydroxypropyl methylcellulose or methyl cellulose, in a sufficient amount to allow reduction in PAA concentration without comprising the in situ gelling properties. In some embodiments, the hydrogel is comprised of Carbopol® 940 and Methocel E50LV (HPMC).

In some embodiments, the hydrogels can include about 15% to about 30% by weight poloxamer, more particularly about 17.5% to about 25%, and even more particularly about 20% to about 25%. In some embodiments, the hydrogels can also include about 0.2% to about 4% by weight carbomer, more particularly about 0.5 to about 2.0%, and even more particularly about 0.7% to about 1.5%.

In some embodiments, the compound is deployed in the hydrogel. In some embodiments, the compound is formulated in a microparticle or nanoparticle, as described herein, and the microparticles and/or nanoparticles deployed in the hydrogel. In some embodiments, the compound of the disclosure is formulated in a liposome, as described herein, and the liposome deployed in the hydrogel. In some embodiments, the compound of the disclosure is formulated in a calcium phosphate particle, as described herein, and the calcium phosphate particle deployed in the hydrogel.

In some embodiments, the pharmaceutical composition of the compound formulated in a hydrogel further comprises one or more excipients selected from a tonicity agent, viscosity enhancing agent, buffering agent, chelating agent, surfactant, preservative, and antioxidant.

In some embodiments, the pharmaceutical composition of the compound formulated in a hydrogel further comprises a tonicity agent. In some embodiments, the pharmaceutical composition of the compound formulated in a hydrogel further comprises a viscosity enhancing agent. In some embodiments, the pharmaceutical composition of the compound formulated in a hydrogel further comprises a buffering agent. In some embodiments, the pharmaceutical composition of the compound formulated in a hydrogel further comprises a chelating agent. In some embodiments, the pharmaceutical composition of the compound formulated in a hydrogel further comprises a surfactant. In some embodiments, the pharmaceutical composition of the compound formulated in a hydrogel further comprises a preservative. In some embodiments, the pharmaceutical composition of the compound formulated in a hydrogel further comprises an antioxidant. In some embodiments, the hydrogel and excipients are suitable for intraocular, e.g., intravitreal administration.

### 4.12. Excipients

In some embodiments, the pharmaceutical compositions comprise one or more pharmaceutically acceptable additive or excipients suitable for injection, including subcutaneous, intradermal, intramuscular, intraperitoneal, intraocular, and in some embodiments, intravenous administration. In particular, the one or more excipients is suitable for intraocular, e.g., intravitreal administration. In some embodiments, the excipient is selected from a tonicity agent, buffering agent, viscosity enhancing agent, chelating agent, surfactant, preservative, and antioxidant.

### 4.12.1. Tonicity Agents

In some embodiments, the pharmaceutical compositions can have one or more tonicity agents, which can be used to adjust the tonicity of the composition suitable for the mode of administration. Suitable tonicity agents include, by way of example and not limitation, dextrans (e.g., dextran 40 or 70), dextrose, glycerin, propylene glycol, and salts, such as potassium or sodium salts. Equivalent amounts of one or more salts made up of cations, for example, such as potassium, ammonium and anions such as chloride, citrate, ascorbate, borate, phosphate, bicarbonate, sulfate, thiosulfate, bisulfate, the salts sodium bisulfate and ammonium sulfate, can also be used.

The amount of tonicity agent will vary, depending on the particular agent to be added. In general, however, the compositions can have a tonicity agent in an amount sufficient to cause the final composition to have a physiologically acceptable osmolarity, such as an ophthalmically acceptable osmolarity. In some embodiments, the cyclodextrin compositions have an osmolarity of about 200 to about 1000 mOsm/L or about 200 to about 500 mOsm/L, or any specific value within said ranges (e.g., 200 mOsm/L, 210 mOsm/L, 220 mOsm/L, 230 mOsm/L, 240 mOsm/L, 250 mOsm/L, 260 mOsm/L, 270 mOsm/L, 280 mOsm/L, 290 mOsm/L, 300 mOsm/L, 310 mOsm/L, 320 mOsm/L, 330 mOsm/L, 340 mOsm/L, 350 mOsm/L, 360 mOsm/L, 370 mOsm/L, 380 mOsm/L, 390 mOsm/L or 400 mOsm/L). In a particular embodiment, the ophthalmic formulations are adjusted with a tonicity agent to an osmolarity of ranging from about 250 to about 450 mOsm/L, or about 250 to about 350 mOsm/L.

### 4.12.2. Antioxidants

In some embodiments, the pharmaceutical compositions include an antioxidant tolerated by the subject or the tissue to be treated, for example intraocular tissues. In some embodiments, suitable antioxidant is glucose, ascorbate, sulfides, superoxide dismutase (SOD), cysteine and derivatives thereof. In some embodiments, the anti-oxidant is sodium bisulfite, sodium metabisulfite, ascorbate, sodium sulfite, or thioglycerol. In some embodiments, other antioxidants, tolerated by the intraocular tissues, known in literature, can be used, e.g. hydrosoluble antioxidants, antioxidants which have at least one —SH or —CHO group, peptides and enzymes.

In some embodiments, the antioxidant is glutathione, N-acetylcysteine, tocopherol, ascorbic acid, tetrahexyldecyl ascorbate, butylated hydroxytoluene (BHT), butylated hydroxyanisole, methylgentisate, L-carnosine, tert-butylhydroquinone (TBHQ), glutathione, including derivatives, combinations, and mixtures thereof. In some embodiments, the antioxidant can be at a concentration of 0.01% to 1% w/v, about 0.02% to 1% w/v; about 0.05% to 1% w/v; about 0.01 to 0.5% w/v; about 0.02% to 0.5% w/v; or about 0.05% to 0.5% w/v. In some embodiments, the pharmaceutical composition is free of antioxidants.

### 4.12.3. Buffering Agents

In some embodiments, the pharmaceutical composition can have one or more buffering agents for adjusting and/or maintaining the pH of the composition. In some embodiments, the one or more buffering agents is used to provide a pH range compatible with the tissue subjected to treatment or administration, for example subcutaneous or intraocular environment. Generally, buffer capacity should be large enough to maintain the product pH for a reasonably long shelf-life but also low enough to allow rapid readjustment of the product to physiologic pH upon administration. Generally, buffer capacities of from about 0.01 to 0.1 can be used, particularly at concentrations that provide sufficient buffering capacity and minimizes adverse effects, e.g., irritation to the tissue. Exemplary buffering agents include, by way of example and not limitation, various salts (e.g., sodium, potassium, etc.), acids or bases, where appropriate, of the following agents, including, among others, acetate, borate, phosphate, citrate, bicarbonate, carbonate, succinate, tartaric, lactic, tetraborate, biphosphate, tromethamine, hydroxyethyl morpholine, or THAM (trishydroxymethylamino-methane).

In some embodiments, the buffering agent can be present from about 0.01 mM to about 100 mM, about 0.05 mM to about 100 mM, about 0.5 mM to about 100 mM, from about 1 mM to about 50 mM, from about 1 mM to about 40 mM, from about 1 mM to about 30 mM, from about 1 mM to about 20 mM, or from about 1 mM to about 10 mM. In some embodiments, the buffering agent can be present at about 0.01 mM, about 0.05 mM, about 0.1 mM, about 0.2 mM, about 0.5 mM, about 1 mM, about 5 mM, about 10 mM, about 20 mM, about 30 mM, about 40 mM, about 50 mM, or about 100 mM.

### 4.12.4. Surfactants

In some embodiments, the pharmaceutical compositions include one or more suitable surfactants, particularly a non-ionic surfactant. In some embodiments, the surfactant is a non-ionic surfactant which is polyol esters, polyoxyethylene esters, poloxamers, polyol esters, such as glycol and glycerol esters and sorbitan derivatives. In some embodiments, the non-ionic surfactant is selected from poly oxyethylene (20) sorbitan monolaurate (Tween 20), polyoxyethylene (20) sorbitan monopalmitate (Tween 40), polyoxyethylene (20) sorbitan monostearate (Tween 60), poly oxyethylene (20) sorbitan mono-oleate (Tween 80), polyoxyethylene (20) sorbitan tristearate (Tween 65), polyoxyethylene (20) sorbitau tri-oleate (Tween 85), sorbitan monopalmitate (Span 40), sorbitan monostearate (Span 60), sorbitan mono-oleate (Span 80), sorbitan tristearate (Span 65), and sorbitan trioleate (Span 8).

In some embodiments, the surfactant is a poloxamer or poloxamine. In some embodiments, the poloxamer is P124, P188, P237, P338, P407 and mixtures thereof (see, e.g., Bermudez et al., 2011, Intl Res J Pharmacy Pharmacol. 1(6):109-118; Xuan et al., 2011, Drug Delivery 18(5):305-31; all publications incorporated herein by reference in their entireties). Poloxamers are also available under the tradename Pluronic®, e.g., L 44 NF, F 68 NF, F 87 NF F 108 NF, and F 127 NF.

In some embodiments, the surfactant is selected from polyoxyethylene 15 hydroxystearate; polyoxy ethylene alkyl ethers, polyoxy ethylene stearates, and polyoxyethylene castor oil, e.g., Cremophor EL, ELP, and RH40. In some embodiments, the non-ionic surfactant is selected from Cremophor EL, Cremophor RH 40, Cremophor RH 60, d-α-tocopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, and mono- and di-fatty acid esters of PEG 300, 400, or 1750.

In some embodiments, the surfactant can be present at about 0.00001% to about 2% w/v; about 0.00005% to about 2% w/v; about 0.0001% to about 2% w/v; about 0.0005% to about 2% w/v; about 0.001% to about 2% w/v; about 0.005% to about 2% w/v; about 0.01% to about 2% w/v; about 0.02% to about 2% w/v; about 0.05% to about 2% w/v; 0.1% to about 2% w/v; about 0.15% to about 2% w/v; about 0.2% to about 2% w/v; about 0.5% to about 2% w/v; or about 1% to about 2% w/v. In some embodiments, the surfactant can be present at about 0.00001% to about 1.5% w/v; about 0.0001% to about 1% w/v; about 0.0005% to about 1% w/v; about 0.001% to about 1% w/v; about 0.005% to about 0.5% w/v; or about 0.01% to about 0.2% w/v.

### 4.12.5. Suspending Agent or Viscosity Enhancing Agent

In some embodiments, the pharmaceutical compositions for injection includes one or more suitable suspending or viscosity enhancing agents. The suspending agents can enhance the physical stability of the compositions, and the viscosity enhancing agents can increase the viscosity of the compositions. Suspending agents and viscosity enhancing agents may overlap. In some embodiments, the suspending agent or viscosity enhancing agent is compatible with the tissue of administration or tissue to be treated.

For example, the vitreous humor contained in the posterior chamber of the eye is viscous. Intravitreal injection of a fluid or suspension of substantially lower viscosity into the posterior segment could therefore result in the presence of two somewhat immiscible phases or layers within the eye, which in turn can lead to the “pooling” of the injected fluid or suspension at the bottom of the posterior chamber and uneven or inconsistent dosing to tissues of the posterior segment.

Suspension of therapeutic compounds in a formulation having a relatively high viscosity, such as one approximating that of the vitreous humor, can limit the uneven distribution of the therapeutic agent. Such viscous formulation comprises a viscosity-inducing component. The therapeutic agent of the present invention may be administered intravitreally as, without limitation, an aqueous injection, a suspension, an emulsion, a solution, a gel or in a sustained release or extended release implant, either biodegradable or non-biodegradable.

In some embodiments, the suspending or viscosity enhancing agent is selected from carbopol, polyvinyl alcohol (PVA), polyvinyl pyrrolidone (PVP), polyvinyl acetate, gelatin, xanthan, gum tragacanth, gum acacia, sodium alginate, and cellulosic derivatives. In some embodiments, cellulosic derivatives include hydroxypropyl methyl cellulose (HPMC), hydroxyethyl cellulose (HEC), and methyl cellulose. In some embodiments, the concentration of suspending or viscosity enhancing agent can be present from about 0.1% to about 2% w/v, about 0.5% to about 1% w/v, or any specific value within the ranges.

In some embodiments, the suspending or viscosity enhancing agent ranges from about 0.1% to about 1.0% w/v, or any specific value within said range (e.g., 0.1-0.2%, 0.2-0.3%, 0.3-0.4%, 0.4-0.5%, 0.5-0.6%, 0.6-0.7%, 0.7-0.8%, 0.8-0.9%, 0.9-1.0%; about 0.2%, about 0.21%, about 0.22%, about 0.23%, about 0.24%, about 0.25%, about 0.26%, about 0.27%, about 0.28%, about 0.29%, about 0.30%, about 0.70%, about 0.71%, about 0.72%, about 0.73%, about 0.74%, about 0.75%, about 0.76%, about 0.77%, about 0.78%, about 0.79%, about 0.80%, about 0.81%, about 0.82%, about 0.83%, about 0.84%, about 0.85%, about 0.86%, about 0.87%, about 0.88%, about 0.89%, or about 0.90%). Where the primary component of the pharmaceutical composition has also viscosity enhancing properties, the suspending agents or viscosity enhancing agents can be used to augment the properties of the pharmaceutical compositions. In some embodiments, the viscosity-inducing component is present in an amount in a range of about 0.5% or about 1.0% to about 5% or about 10% or about 20% (w/v) of the composition.

### 4.12.6. Preservatives

In some embodiments, the pharmaceutical compositions can have one or more suitable preservatives, for example, to extend shelf life or limit bacterial growth in the compositions during storage as well as when administered to a subject. Preservatives that can be used, include, among others, benzalkonium chloride, benzethonium chloride, benzododecinium bromide, cetylpyridinium chloride, chlorobutanol, ethylenediamine tetracetic acid (EDTA), thimerosal, phenylmercuric nitrate, phenylmercuric acetate, methyl/propylparabens, phenylethyl alcohol, sodium benzoate, sodium propionate, sorbic acid, and sodium perborate. The amount of preservative in the solution can be a level that enhances the shelf life, limits bacterial growth, or otherwise preserves the composition (e.g., ophthalmic composition, subcutaneous, etc.) with minimal toxicity to the affected tissues (see. e.g., The United States Pharmacopeia, 22nd rev., and The National Formulary, 17th Ed. Rockville, Md.). Levels of preservative suitable for use in the compositions herein can be determined by the person skilled in the art. In some embodiments, the preservatives can be used at an amount of from about 0.001% to about 1.0% w/v. For example, the preservative is present from about 0.005% to about 0.05% w/v, 0.005% to about 0.04% w/v, 0.01% to about 0.03% w/v, 0.01% to about 0.02% w/v, or from about 0.01% to about 0.015% w/v. In some embodiments, the amount of preservative can be about 0.005% w/v, about 0.01% w/v, about 0.012% w/v, about 0.014% w/v, about 0.016% w/v, about 0.018% w/v, about 0.02% w/v, about 0.03% w/v, about 0.04% w/v, or about 0.05% w/v. In some embodiments, no preservatives are used in the compositions.

The pharmaceutical compositions described herein can be prepared by the guidance provided herein and standard techniques, such as described in Remington: The Science and Practice of Pharmacy, 21st Ed. (2005).

### 4.13. Disorders and Diseases for Treatment

In another aspect, the pharmaceutical compositions are used for the treatment of diseases, disorders, or conditions characterized by the presence of toxic aldehydes. As used herein, the term “treating” or “treatment” of a disease, disorder, or condition includes (i) preventing the disease, disorder, or syndrome from occurring in a subject, i.e., causing the clinical symptoms of the disease, disorder, or syndrome not to develop in a subject that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition can be made and determined by one of ordinary skill in the art, particularly in view of the guidance provided in the present disclosure.

In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment may also continue after symptoms have resolved, for example to prevent, delay, or lessen the severity of their recurrence.

In the embodiments for treatment of a disease, disorder, or condition, a subject in need of treatment is administered a therapeutically effective amount of the pharmaceutical composition comprising a compound described herein. The term “therapeutically effective amount” refers to that amount which, when administered to a subject for treating a disease, disorder, or condition is sufficient to effect such treatment for the disease, disorder, or condition.

In some embodiments, a pharmaceutical composition of the disclosure is administered to a subject in thereof in an effective amount to reduce the risk of or for prevention of a disease, disorder or condition characterized by an increase or elevated level of a toxic aldehyde. In some embodiments, a subject in need thereof of is administered a prophylactically effective amount of pharmaceutical composition of the disclosure to reduce the risk of or for prevention of a disease, disorder or condition characterized by an increase or elevated level of a toxic aldehyde.

In some embodiments, the disease, disorder, or condition is characterized by an increase in or elevated level of an aldehyde, such as malondialdehyde (MDA), 4-hydroxynonenal (HNE), frans-2-heptenal, irani-2-nonenal, acrolein, aminoadipic semialdehyde (AASA), retinaldehyde (in the eye), acetaldehyde, glyoxal, methylglyoxal, succinic semialdehyde, and/or phosphatidylcholine γ-hydroxyalkenal (PC-HA) in affected cells, tissues, or organs. In some embodiments, the increase in or elevated level of the aldehyde is at least 1.1-fold, 1.2-fold, 1.3-fold, 1.4-fold, 1.5-fold, 2-fold, 2.5-fold, 5-fold, 10-fold as compared to that in normal cells, tissues or organs. The compounds described herein can decrease aldehyde (e.g., MDA and HNE) concentrations in a concentration-dependent manner. The amount or concentration of aldehydes (e.g., MDA, HNE, and/or acrolein) can be measured by methods or techniques known in the art, such as those described in Tukozkan et al., 2006, Furat Tip Dergisi 11: 88-92, Grotto et al., 2009, Quim. Nova 32(1)1, and Rauli et al., 1998, 1 Biochem. 123:918-923, incorporated herein by reference.

### 4.13.1. Treatment of Ocular Disorders

In some embodiments, the compositions are used to treat, reduce the risk of, or to prevent an ocular disease, disorder or condition characterized by an increase or elevated level of toxic aldehydes. In some embodiments, the ocular disease, disorder, or condition is characterized by the presence of a pathological inflammatory response. In some embodiments, a method of treating, reducing the risk of or preventing the occurrence of an ocular disease, disorder or condition characterized by an increase or elevated level of toxic aldehydes comprises administering to a subject in need thereof a therapeutically effective amount of a pharmaceutical composition of the disclosure. In some embodiments, the pharmaceutical composition is administered intraocularly, for example intravitreally.

In some embodiments, the ocular disease, disorder or condition for treatment or for prevention includes macular degeneration, including age-related macular degeneration (AMD), wet age-related macular degeneration, dry age-related macular degeneration; Stargardt's disease; dry eye syndrome or disease; cataracts; keratoconus; bullous and other keratopathy; Fuch's endothelial dystrophy; allergic conjunctivitis; ocular cicatricial pemphigoid; lacrimal gland dysfunction; uveitis, including anterior uveitis, posterior uveitis, and pan-uveitis; scleritis; ocular Stevens-Johnson Syndrome; ocular rosacea, with or without meibomian gland dysfunction; macular edema, including diabetic macular edema (DME), non-clinically significant macular edema (Non-CSME), and clinically significant macular edema (CSME); endophthalmitis; and inflammation and/or fibrosis associated with ocular injury.

In some embodiments, the ocular disease, disorder, or condition for treatment with the pharmaceutical compositions is inflammation and/or fibrosis associated with ocular injury, such as from trauma to the eye or surgery, including corneal fibrosis. In some embodiments, the ocular disease, disorder, or condition for treatment with the pharmaceutical composition is traumatic eye injury; cataract surgery; laser eye surgery, including penetrating keratoplasty (PK), phototherapeutic keratectomy (PTK), and corneal transplant surgery; refractive surgery, including photorefractive keratectomy (PRK), laser thermal keratoplasty (LTK), and radial keratotomy (RK); and vitreoretinal surgery, including vitrectomy, retinal detachment repair, and posterior sclerotomny.

In some embodiments, the pharmaceutical compositions are used to treat an ocular disease, disorder, or condition characterized by an increase or elevated level of a toxic aldehyde, where the ocular disease, disorder, or condition affects the posterior segment of the eye (i.e., back of the eye). A condition of the posterior segment (posterior ocular condition) of the eye is a disease, disorder or condition which significantly affects or involves a tissue or cell type in a posterior ocular region or site (that is, in a position posterior to a plane through the posterior wall of the lens capsule), such as the parts of the choroid or sclera, vitreous, vitreous chamber, retina, optic nerve (e.g., the optic disc), and blood vessels and nerves which vascularize or innervate a posterior ocular (or posterior segment) region or site.

In some embodiments, a posterior segment or posterior ocular disease, disorder or condition can be selected form macular degeneration, such as age-related macular degeneration, wet age-related macular degeneration; acute macular neuroretinopathy; macular edema, such as cystoid macular edema or diabetic macular edema; diabetic retinopathy (including proliferative diabetic retinopathy); posterior uveitis; pan-uveitis, Stargardt's disease, ocular trauma which affects a posterior ocular site or location; a posterior ocular condition caused by or influenced by an ocular laser treatment, photodynamic therapy, photocoagulation, radiation retinopathy, and glaucoma.

In some embodiments, the pharmaceutical compositions are used to treat an ocular disease, disorder, or condition characterized by an increase or elevated level of a toxic aldehyde, where the ocular disease, disorder, or condition affects the anterior segment of the eye (i.e., front of the eye). An “anterior ocular condition” is a disease, disease or condition which affects or which involves an anterior (i.e., front of the eye) ocular region or site, such as a periocular muscle, an eye lid or an eye ball tissue or fluid which is located anterior to the posterior wall of the lens capsule or ciliary muscles. An anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

A front of the eye ocular condition can include a disease, disorder or condition such as, for example, conjunctivitis, allergic conjunctivitis; dry eye syndrome; anterior uveitis, pan-uveitis, lacrimal apparatus diseases; lacrimal duct obstruction, Fuch's endothelial dystrophy, and Stevens-Johnson syndrome.

### 4.13.2. Treatment of Non-Ocular Disorders

In some embodiments, the pharmaceutical compositions are used to treat non-ocular diseases, disorders, or conditions characterized by the presence of toxic aldehydes. In some embodiments, non-ocular diseases, disorders, or conditions having increased levels of toxic aldehydes include inflammatory diseases, disorders, or conditions; autoimmune diseases or disorders; neurodegenerative diseases or disorders; chronic liver diseases or disorders; chronic obstructive lung disease or disorders; and non-ocular fibrotic diseases or disorders.

In some embodiments, the non-ocular disorder for treatment with the pharmaceutical compositions include psoriasis, topical (discoid) lupus, contact dermatitis, atopic dermatitis, allergic dermatitis, radiation dermatitis, acne vulgaris, Sjögren-Larsson Syndrome (SLS) and/or its associated ichthyosis, succinic semialdehyde dehydrogenase deficiency (SSADH) deficiency, solar elastosis/wrinkles, dermal injury, dermal thermal injury, chemical burns or injury, and scleroderma.

In some embodiments, the disease, disorder or condition for treatment with the pharmaceutical compositions is chronic liver diseases or disorders. In some embodiments, the chronic liver disease is non-alcoholic fatty liver disease (NAFLD), non-alcoholic steatohepatitis (NASH), primary biliary cirrhosis, primary sclerosing cholangitis.

In some embodiments, the disease, disorder or condition for treatment with the pharmaceutical compositions is a chronic obstructive lung disease. In some embodiments, the disease, disorder or condition is asthma and chronic obstructive pulmonary disease (COPD).

In some embodiments, the disease, disorder, or condition for treatment with the pharmaceutical compositions is an inflammatory disease, disorder, or condition. In some embodiments, the disease, disorder or condition is selected from inflammatory bowel disease (IBD), sepsis, atherosclerosis, ischemic-reperfusion injury, diabetes (e.g., Type 2), Crohn's disease, ulcerative colitis, irritable bowel syndrome, elastosis, ankylosing spondylitis, osteoporosis, sarcoidosis, rheumatoid arthritis (RA), osteoarthritis, psoriatic arthritis, atherosclerosis, pulmonary arterial hypertension, lupus nephritis, diabetic neuropathy, pre-eclampsia, and fibrotic disease, such as renal, hepatic, pulmonary, chronic kidney disease, eosinophilic esophagitis, and cardiac fibrosis.

In some embodiments, the disease, disorder, or condition for treatment with the pharmaceutical compositions is an autoimmune disease, disorder, or condition. In some embodiments, the disease, disorder, or condition is selected from diabetes (e.g., Type 1), lupus, rheumatoid arthritis, lupus, systemic lupus erythematosis, psoriasis, and scleroderma,

In some embodiments, the disease, disorder, or condition for treatment with the pharmaceutical compositions is a chronic neurological disease. In some embodiments, the disease, disorder, or conditions is selected from Parkinson's disease, Alzheimer's disease, Huntington's disease, multiple sclerosis, pyridoxine-dependent epilepsy, neurogical and/or motor neuron effects of SLS, neurogical and/or motor neuron effects of SSADH deficiency, and amyotrophic lateral sclerosis.

In some embodiments, the disease, disorder or conditions for treatment with the pharmaceutical compositions is a genetic condition having a defect in aldehyde metabolism that results in an increase or elevated level of a toxic aldehyde. In some embodiments, the disease, disorder or condition is Sjögren-Larsson Syndrome or succinic semialdehyde dehydrogenase (SSADH) deficiency.

A skilled person would understand that the disease, disorder, or condition listed herein may involve more than one pathological mechanism. For example, a disease, disorder, or condition listed herein may involve dysregulation in the immunological response and/or inflammatory response. Thus, the above categorization of a disease, disorder, or condition is not absolute, and the disease, disorder, or condition may be considered an immunological, an inflammatory, a cardiovascular, a neurological, and/or metabolic disease, disorder, or condition.

### 4.14. Administration and Dosages

In various embodiments, the pharmaceutical compositions are administered by injection. In some embodiments, the pharmaceutical compositions are administered parenterally. In some embodiments, the pharmaceutical compositions disclosed herein can be “locally administered,” that is administered at or in the vicinity of the site at which a therapeutic result or outcome is desired. In some embodiments, the pharmaceutical compositions are systemically administered, that is for distribution throughout the body. As discussed herein, the pharmaceutical compositions can be administered subcutaneously, intradermally, intramuscularly, intraperitoneally, intraocularly, intraarticularly, and intra-lesionaly, or wherein appropriate and suitable for the composition, intravenously.

In some embodiments for treating ocular disorders, the pharmaceutical composition is administered intraocularly, including intravitreal, subconjunctival or periocular injection. In some embodiments, for treating diseases, disorders, or conditions affecting the back of the eye, the pharmaceutical composition is administered into the posterior segment of the eye. In some embodiments for intraocular administration, the pharmaceutical compositions are administered to provide a depot of the pharmaceutical composition. For example, to treat an ocular condition such as for example a macular edema, or macular degeneration or uveitis, intravitreal injection or implantation of a pharmaceutical composition of the compound formulated as a viscous composition can be carried out and is an example of local administration.

In various embodiments, the dosage administered is an amount effective to treat the disease or disorder. As noted above, the dose administered can take into consideration the compound, age, body weight, general health, sex, diet, time of administration, mode of administration, drug interaction and the disease, disorder, or condition being treated and its severity.

In some embodiments, the pharmaceutical compositions containing the compounds are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention can be determined by the attending physician within the scope of sound medical judgment.

In some embodiments, the compound, as part of a pharmaceutical composition, is administered parenterally at dosage level of about 0.01 mg/kg to about 100 mg/kg, about 0.01 mg/kg to about 50 mg/kg, and preferably from about 1 mg/kg to about 25 mg/kg, of subject body weight per day, one or more times a day, to obtain the desired therapeutic effect.

In some embodiments, for local administration, the compound is administered at a dosage level of about 0.001 mg to about 5 mg or more, about 0.002 mg to about 5 mg, about 0.005 mg to about 5 mg, about 0.01 mg to about 5 mg, about 0.02 mg to about 5 mg, about 0.05 mg to about 5 mg, about 0.1 mg to about 5 mg, about 0.2 mg to about 5 mg, about 0.5 mg to about 5 mg, about 1 mg to about 5 mg, or about 2 mg to about 5 mg. In some embodiments, for local administration, the compound is administered at a dosage level of about 0.001 mg, 0.002 mg, 0.005 mg, 0.01 mg, 0.02 mg, 0.05 mg, 0.1 mg, 0.2 mg, 0.5 mg, or 1 mg, 2 mg, 3 mg, 4 mg, 5 mg or more.

In some embodiments, the pharmaceutical composition is administered intraocularly, for example by intravitreal injection, for treating certain ocular disorders. In some embodiments for intraocular administration, the composition is administered into the eye using a syringe. In some embodiments, the composition can be administered into the eye during eye surgery, such as vitrectomy.

In some embodiments, the volume administered intraocularly is from about 1 μL to about 100 uL, at least about 5 uL to about 100 uL, at about 10 μL to about 100 uL; at about 20 μL to about 100 uL, at about 30 uL to about 100 uL; at about 40 uL to about 100 uL, at about 50 uL to about 100 uL; at about 60 uL to about 100 uL; at about 70 uL. In some embodiments, the volume administered can be up to 4 mL, depending on the mode of administration. In some embodiments, the volume administered can be up to 3 mL, up to 2.5 mL, up to 2 mL, up to 1.5 mL, or up to 1 mL. In some embodiments, the volume administered to the eye is less than 1 mL. In some embodiments, the volume administered intraocularly is from about 1 μL to about 95 μL, at least about 5 uL to about 90 uL, about 10 μL to about 85 uL; about 20 μL to about 80 uL; about 30 uL to about 75 uL; or about 40 uL to about 70 uL.

In some embodiments, the volume administered intraocularly is at least about 1 μL, at least about 5 uL, at about 10 μL; at about 20 μL, at about 30 uL; at about 40 uL, at about 50 uL; at about 60 uL; at about 70 uL; at about 80 uL; at about 90 uL; or at about 100 μL.

In some embodiments, the compound may be administered daily, more than once daily, twice a week, three times a week, weekly, biweekly, monthly, bimonthly, semiannually, annually, and/or biannually.

The following examples are provided to further illustrate the methods of the present disclosure, and the compounds and compositions for use in the methods. The examples described are illustrative purposes only and are not intended to limit the scope of the invention in any way.

### 5. EXAMPLES

As depicted in the Examples below, in certain exemplary embodiments, compounds can be used to treat diseases, disorders, or conditions characterized by the presence of aldehydes according to the following procedures. It will be appreciated that, although the methods depict use of an exemplary compound, the following general methods can be applied to other compounds and its subclasses and species, as described herein.

### Example 1: Effect of Compound I-5 and I-22 on Rat Endotoxin Induced Uveitis

An in vivo study was conducted to assess the efficacy of intravitreal administration of Compound I-5 and I-22 in a rat model of endotoxin induced uveitis, one of the most appropriate models for the study of non-infectious uveitis (NIU) (Smith et al., 1998, Immunol Cell Biol. 76(6):497-512) (Toxikon Study 16-04078-N1). Ocular inflammation was induced in female Lewis rats (n=10/group) by a single, foot pad injection of lipopolysaccharide (LPS) (100 μL) (Herbort et al., 1988, Graefes Arch Clin Exp Ophthalmol. 226(6):553-8).

Compound I-5 or I-22 (50 μg) was topically administered (TO) to the eye at hours 1, 3, 7, 10, and 17, after LPS induction, or by a single intravitreal (IVT) injection (25 μg) 1 hour after LPS induction (n=10 per group). Ocular exams were performed six and 24 hours after LPS injection. Anterior segments were scored using a combined Draize and McDonald-Shattuck scoring system, and posterior segments were scored using 0 to 1 (vitreous, optic disc, retinal vasculature) and 0 to 4 (retinal and choroidal hemorrhage, exudation, and detachment) scales. Statistical significance from vehicle control was determined by ANOVA, followed by Tukey's post hoc test. The test compound, e.g., Compound I-22 (5 mg/mL), was administered intravitreally into each eye (25 μg/eye) within one hour of LPS administration. Balanced salt solution (BSS) served as a vehicle control. Retinal exams were performed prior to study start and six and 24 hours following LPS administration and scored using a Combined Draize and McDonald Shadduck scoring system, based on assessments of: (a) Retinal vasculopathy; (2) Retinal hemorrhage, exudate or detachment; and (3) Retinal hemorrhage, exudate or detachment.

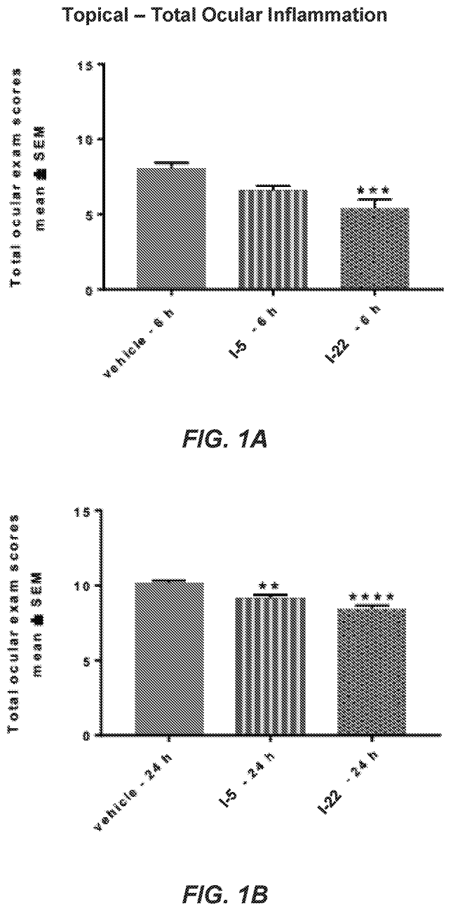

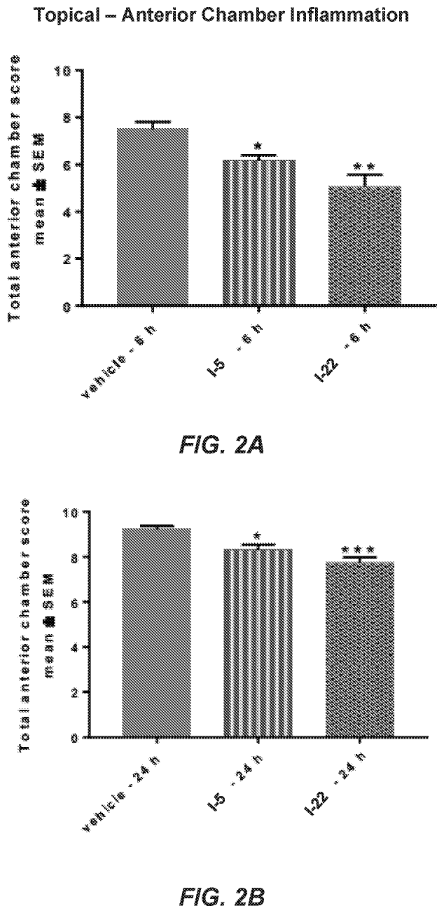

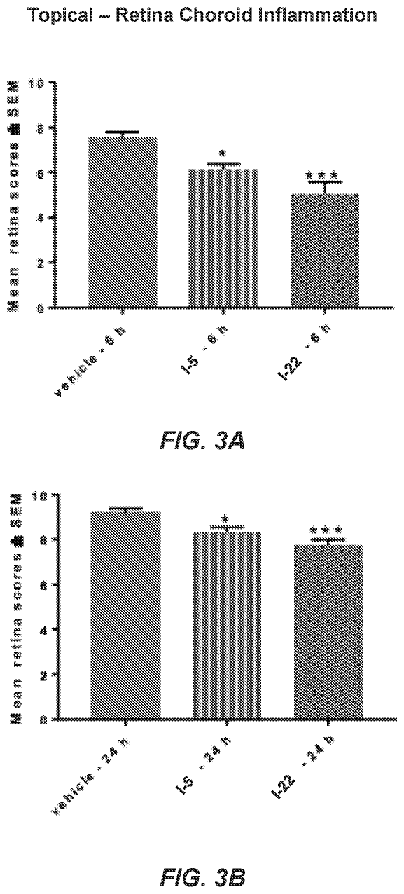

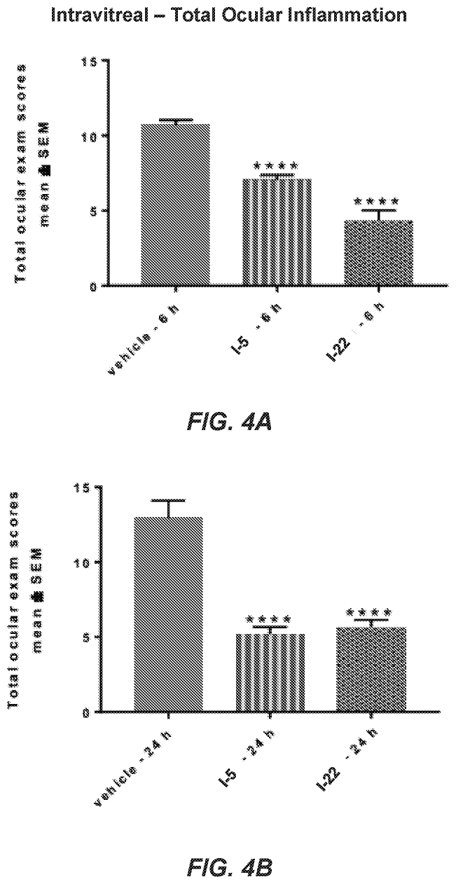

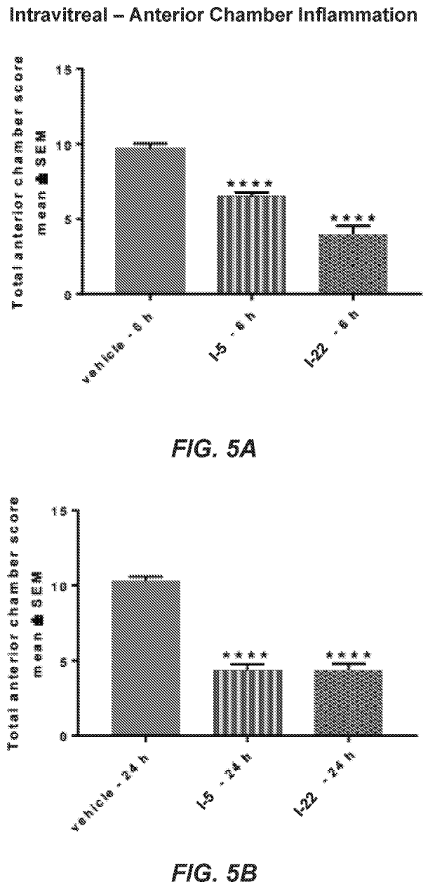

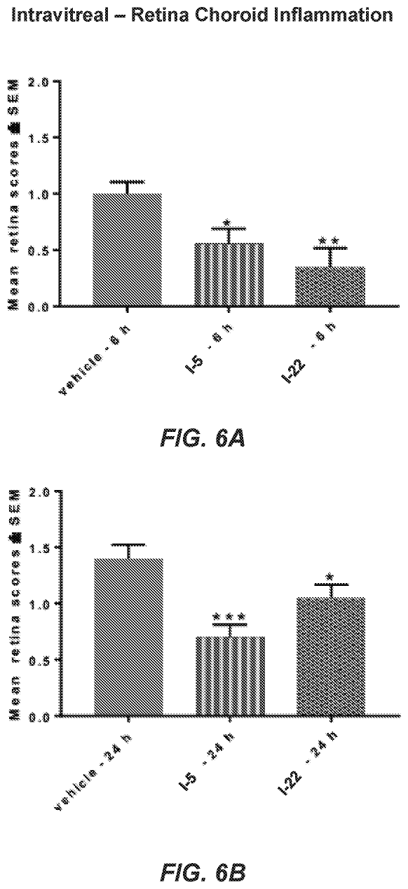

In the LPS-induced uveitis model, ocular exam scores were significantly improved, compared to vehicle, at 6 hours and 24 hours after topical (TO) administration of Compound I-5 or Compound I-22 (Total Ocular Inflammation—FIG. 1A and FIG. 1B; Anterior Chanber Inflammation—FIG. 2A and FIG. 2B; and retina-choroid inflammation—FIG. 3A and FIG. 3B). After intravitreal (IVT) administration of Compound I-5 or Compound I-22, ocular exam scores were also significantly improved vs. vehicle (Total Ocular Inflammation—FIG. 4A and FIG. 4B; Anterior Chanber Inflammation—FIG. 5A and FIG. 5B; and retina-choroid inflammation—FIG. 6A and FIG. 6B). Small, although not significant, reductions in MDA adducts were observed in the Compound I-5 and Compound I-22 groups after TO and IVT administration (data not shown). To control for variations between individual animals, a within-subject statistical analysis, examining the covariates of treatment and time, was conducted on the retina-choroid scores from the IVT groups. Retina-choroid scores in rats were significantly improved following IVT treatment with either Compound I-5 or Compound I-22 compared to vehicle control.

### Example 2: Effect of Compound I-5 and I-22 on Formation of N-retinylidene-N-retinylethanolanmine (A2E)

A mouse knockout model (abcr−/−) of macular degeneration (MD) was used to test the activities of Compound I-5 and Compound I-22. The retinal transport protein, ABCA4, is not expressed in these mice, allowing retinaldehdye to escape the light cycle and form a toxic metabolite with phosphatidylethanolamine, N-retinylidene-phosphatidylethanolamine (A2E). Mice were treated intraperitoneally (IP) for 56 days with 10 mg/kg Compound I-5 (n=24), Compound I-22 (n=24), or vehicle (n=22), starting at week 10 to 12 of life. An untreated control group (n=12) was sacrificed on the first day of dosing. After treatment, A2E concentrations in retinas were measured by HPLC. Statistical significance from vehicle control was determined by t-test.

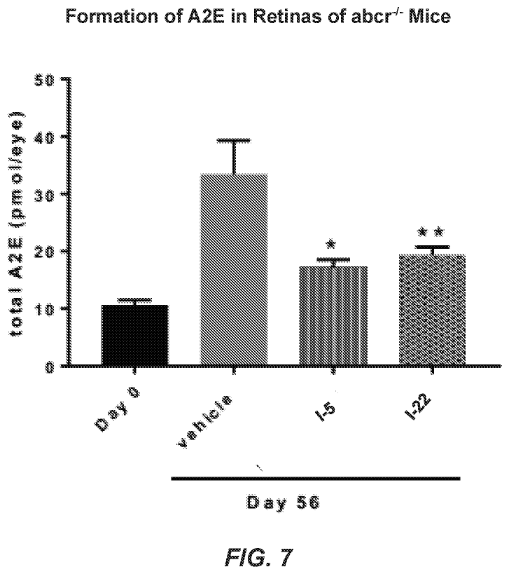

In the MD model, daily Compound I-5 or Compound I-22 treatment decreased formation of A2E by 71% or 69%, respectively, compared with vehicle controls (FIG. 7). In a separate study, systemic (IP) doses of Compound I-5, 5-fold greater than the effective dose in the A2E study, administered for 56 days, had no effect on dark adaptation (data not shown). Daily treatment with Compound I-5 or Compound I-22 for 56 days had no effect on body weight, nor were any drug-related adverse effects observed.

### Example 3: Effect of Compound I-22 on Diabetic Macular Edema

Diabetic macular edema (DME) is a common cause of vision loss. Hyperglycemia can lead to carbonyl stress in the retina, resulting in accumulation of toxic aldehydes such as methylglyoxal, 4-hydroxy-trans-2-nonenal, and malondialdehyde, which induce inflammatory changes in the eye, including the development of DME.

To assess the effect of Compound I-22 in a rat model of diabetic macular edema (DME), Type 1 diabetes was induced in male brown Norway rats by intraperitoneal administration of streptozotocin (STZ; 55 mg/kg). Forty days after STZ administration, animals were tested for diabetes by testing blood glucose levels.

Animals with diabetes were assigned to groups of ten, to receive either vehicle (HPβCD) or Compound I-22. A group of four animals that did not receive STZ or drug treatment served as non-diabetic controls. On Day 41 (about Week 6) after STZ induction, animals received a single intravitreal injection of Compound I-22 (3.5 μL; 17.5 μg) or vehicle per eye. On Day 57 (about Week 8) after STZ induction, animals received a second intravitreal injection of Compound I-22 or vehicle.

On Day 70 (about Week 10) after STZ induction, animals were euthanized and retinas were processed for histological examination. Sections (3 to 5 μm) were stained with hematoxylin and eosin and evaluated by light microscopy. Changes in retinal thickness were scored on a 0 to 4 scale, with 0 meaning no changes, 1 meaning minimal changes, 2 meaning mild changes, 3 meaning moderate changes, and 4 meaning severe changes, relative to non-diabetic controls. Six microscopic sections were examined for each eye, and retinas were also assessed and scored by a pathologist for changes in vascularity and neutrophil infiltration.

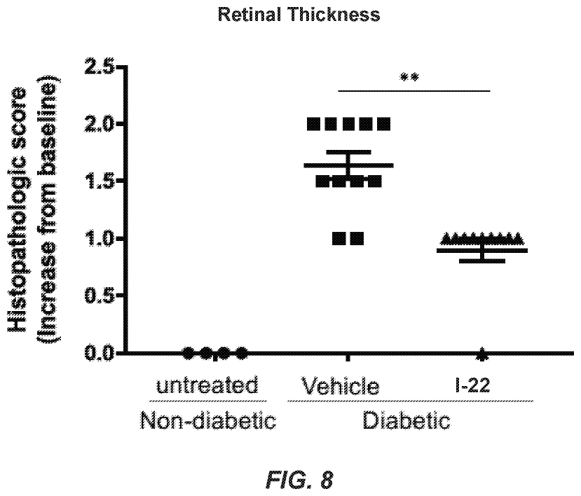

Results. Retinal thickness in the STZ-induced animals was significantly greater than in control animals, as illustrated in FIG. 8. When STZ-induced animals were treated with Compound I-22, there was a significant reduction in retinal thickness compared to the retinal thickness of untreated STZ-induced animals. The scoring used the following: 0=normal, 1=minimal microscopically visible changes; 2=mild microscopically visible changes; 3=moderate microscopically visible changes, 4=severe microscopically visible changes. **p<0.01 Statistical analysis was performed by a non-parametric Dunn's multiple comparison followed by the Kruskal-Wallis test.

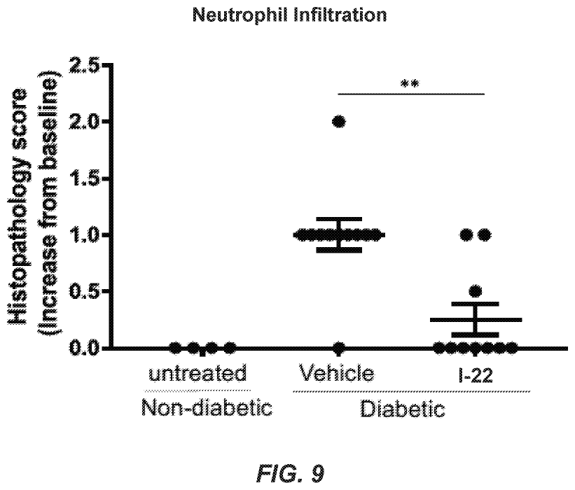

The decrease in retinal thickness was accompanied by a significant reduction in neutrophil infiltration compared to vehicle based on assessment of microscopic sections of the retinas as shown in FIG. 9. Scoring is based on the following: 0=normal, 1=minimal microscopically visible changes, 2=mild microscopically visible changes, 3=moderate microscopically visible changes, 4=severe microscopically visible changes. **p<0.01 Statistical analysis was performed by a non-parametric Dunn's multiple comparison followed by the Kruskal-Wallis test.

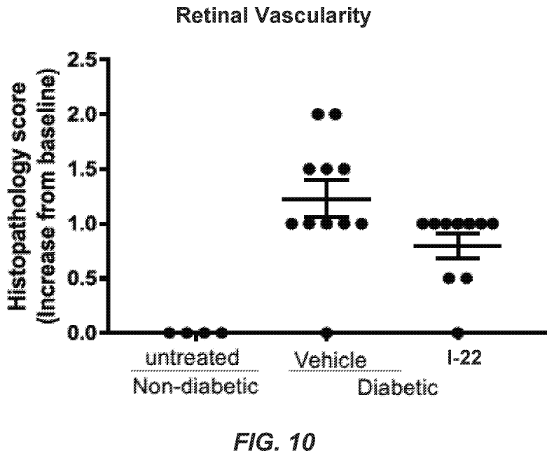

Microscopic sections of the retinas were also assessed for vascular leakage. Treatment with Compound I-22 inhibited diabetes-induced retinal vascular changes, as indicated by a decrease in retinal vascularity score by 36% (p<0.05) in the Compound I-22-treated group compared to vehicle. However, the reduction in retinal vascularity compared to vehicle was not statistically significant (see FIG. 10). Retinal vascularity was based on the following scoring: 0=normal, 1=minimal microscopically visible changes, 2=mild microscopically visible changes, 3=moderate microscopically visible changes, 4=severe microscopically visible changes.

Summary. Diabetic retinopathy was successfully induced in STZ-treated rats. The diabetic animals lost approximately 10% of body weight in the first week post STZ administration and maintained the weight until the end of study. Non-fasting glucose blood levels were significantly elevated in the diabetic rats compared to non-diabetic rats.

Increases in retinal thickness, vascularity, and neutrophil infiltration were observed in the vehicle-treated diabetic rats compared to the non-diabetic rats. Compound I-22 treatment decreased retinal inflammation, as measured by statistically significant decreases in retinal thickness, neutrophil infiltration, and retinal vascular changes.

In conclusion, the data suggest that sequestration of aldehydes represents a novel therapeutic approach for the treatment of the ophthalmic inflammatory sequelae of diabetes. Compound I-22 decreased retinal inflammation, blocked neutrophil infiltration and inhibited retinal vascular changes in this model of DME. Compound I-22 was also well tolerated in the retina.

The data suggest that Compound I-22 would be useful in the treatment of retinal diseases such as DME, particularly by intravitreal administration.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

We claim:

1. A pharmaceutical composition comprising:
a compound of formula XVII-B: 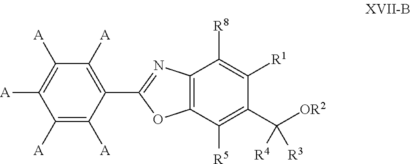
or a pharmaceutically acceptable salt thereof, wherein:
each A is independently selected from hydrogen and deuterium;
R¹ is selected from —NH₂, —NHD, and —ND₂;
R² is selected from hydrogen and deuterium;
R³ and R⁴ are independently selected from —CH₃, —CH₂D, —CHD₂, and —CD₃; and
R⁵ and R⁸ are each independently selected from hydrogen and deuterium,
or a pharmaceutically acceptable salt thereof, and
wherein the pharmaceutical composition is an injectable composition comprising:
(a) a viscosity enhancing agent;
(b) a liposome;
(c) a microparticle or nanoparticle of biodegradable polymer;
(d) a calcium phosphate particle;
(e) a cyclodextrin, wherein the composition is suitable for parenteral administration; or
(f) a hydrogel.

2. (canceled)

3. (canceled)

4. The pharmaceutical composition of claim 1, comprising the viscosity enhancing agent and wherein the viscosity enhancing agent is selected from the group consisting of hyaluronate, hyaluronic acid, cross-linked hyaluronic acid, polyvinylpyrrolidone (PVP), hydroxypropyl methylcellulose, hydroxypropyl cellulose, hydroxylethyl cellulose, glycerol, or mixtures thereof.

5. The pharmaceutical composition of claim 4, wherein the pharmaceutical composition has a viscosity of between 1 kcP and 200 kcP.

6. The pharmaceutical composition of claim 5, wherein the viscosity enhancing agent is hyaluronate, hyaluronic acid or a pharmaceutically acceptable salt thereof.

7. The pharmaceutical composition of claim 6, wherein the hyaluronate or hyaluronic acid or a pharmaceutically acceptable salt thereof has a molecular weight of about 500,000 to about 5×10⁶ Daltons.

8. The pharmaceutical composition of claim 6, further comprising one or more excipients selected from a tonicity agent, buffering agent, chelating agent, surfactant, preservative, and antioxidant.

9. The pharmaceutical composition of claim 1, comprising a liposome.

10-12. (canceled)

13. The pharmaceutical composition of claim 9, wherein the liposome comprises egg phosphatidylcholine (EPC) and l-α-distearoyl phosphatidylcholine (DSPC).

14. The pharmaceutical composition of claim 9, wherein the liposome comprises egg phosphatidylcholine (EPC) or 1,2-dipalmitoyl-sn-glycero-3-phosphocholine, (DPPC), palmitoyl-oleoylphosphatidylcholine (POPC), and cholesterol or a derivative thereof.

15. The pharmaceutical composition of claim 9, wherein the liposome comprises dioleoylphosphatidylcholine (DOPC), dioleoylphosphatidylglycerol (DOPG), and cholesterol.

16-20. (canceled)

21. The pharmaceutical composition of claim 9, wherein the liposome is surface modified with poly-L-lysine.

22. (canceled)

23. The pharmaceutical composition of claim 1, comprising a microparticle or nanoparticle of biodegradable polymer, and wherein the biodegradable polymer comprises poly(ester)s, poly(ester amide)s, poly(anhydride)s, poly(carbonate)s, poly(amino acid)s, poly(amide)s, poly(urethane)s, poly(ortho-ester)s, poly(iminocarbonate)s, poly(phosphazene)s, or combinations thereof.

24. The pharmaceutical composition of claim 23, wherein the biodegradable polymer comprises poly(lactide) (PLA), poly(lactide-co-glycolide) (PLGA), polyglycolide (PGA), polyhydroxybutyric acid, polycaprolactone, polyvalerolactone, polyphosphazene, polyorthoester, or combinations thereof.

25-33. (canceled)

34. The pharmaceutical composition of claim 1, comprising a calcium phosphate particle.

35. (canceled)

36. (canceled)

37. The pharmaceutical composition of claim 34, wherein the calcium phosphate particle has a surface coating of polyethylene glycol.

38. (canceled)

39. The pharmaceutical composition of claim 1, comprising a complexing agent selected from a polyamino acid, galactomannan or cationic galactomannan polymer, cellulosic polymer, quaternary ammonium polymer, and combinations thereof.

40-44. (canceled)

45. The pharmaceutical composition of claim 1, further comprising a cyclodextrin, wherein the cyclodextrin is selected from α-cyclodextrin, β-cyclodextrin, γ-cyclodextrin, derivatives thereof, and combinations thereof.

46. The compound of claim 45, wherein the cyclodextrin is selected from carboxyalkyl cyclodextrin, hydroxyalkyl cyclodextrin, sulfoalkylether cyclodextrin, and alkyl cyclodextrin.

47. (canceled)

48. The compound of claim 45, wherein the cyclodextrin is a β-cyclodextrin selected from hydroxyalkyl-β-cyclodextrin and sulfoalkylether-β-cyclodextrin.

49. The compound of claim 48, wherein the cyclodextrin is present at about 0.2% to about 15% w/v.

50-54. (canceled)

55. The pharmaceutical composition of claim 1, further comprising a hydrogel, wherein the hydrogel comprises polyethylene oxide; polypropylene oxide; polyacrylic acid (PAA); polyvinylpyrrolidone; polyethylene glycol (PEGs); gelatin, polyvinyl alcohols (PVA); polyhydroxyethyl methacrylate (poly-HEMA or PHEMA); cellulose; alginate; chitin; or combinations thereof.

56. The pharmaceutical composition of claim 55, wherein the hydrogel is selected from poloxamer 407, poloxamer 188, and mixtures thereof.

57. (canceled)

58. The pharmaceutical composition of claim 55, wherein the hydrogel comprises Carbopol® 974P.

59-63. (canceled)

64. The pharmaceutical composition of claim 1, wherein the compound is 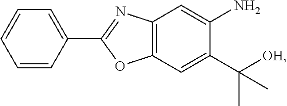 [SMILES: CC(C)(O)c1cc2oc(-c3ccccc3)nc2cc1N]
or a pharmaceutically acceptable salt thereof.

65. (canceled)

66. A method of treating a disease, disorder, or condition selected from an ocular disorder selected from macular degeneration, including age-related macular degeneration (AMD), wet age-related macular degeneration, dry age-related macular degeneration; Stargardt's disease; dry eye syndrome or disease; cataracts; keratoconus; bullous and other keratopathy; Fuch's endothelial dystrophy; allergic conjunctivitis; ocular cicatricial pemphigoid; lacrimal gland dysfunction; uveitis, including anterior uveitis, posterior uveitis, and pan-uveitis; scleritis; ocular Stevens-Johnson Syndrome; ocular rosacea, with or without meibomian gland dysfunction; macular edema, including diabetic macular edema (DME), non-clinically significant macular edema (Non-CSME), and clinically significant macular edema (CSME); endophthalmitis; and inflammation and/or fibrosis associated with ocular injury.

67. The method of claim 66, wherein the ocular disease, disorder, or condition is inflammation and/or fibrosis associated with ocular injury.

68. The method of claim 67, wherein the ocular injury is traumatic eye injury; cataract surgery; laser eye surgery, including penetrating keratoplasty (PK), phototherapeutic keratectomy (PTK), and corneal transplant surgery; refractive surgery, including photorefractive keratectomy (PRK), laser thermal keratoplasty (LTK), and radial keratotomy (RK); and vitreoretinal surgery, including vitrectomy, retinal detachment repair, and posterior sclerotomy.

69. (canceled)

70. The pharmaceutical composition of claim 1, wherein each A is deuterium.

71. The pharmaceutical composition of claim 1, wherein each A is deuterium; R¹ is —NH₂, R² is hydrogen, R³ and R⁴ are —CH₃, R⁵ is hydrogen, and R⁸ is hydrogen.
